(19) United States
(12) Patent Application Publication
(10) Pub. No.: US 2011/0099668 A1
(43) Pub. Date: Apr. 28, 2011

(54) Expressing GLK in plants

(76) Inventors: Jasbir Singh, Ottawa (CA); Ghislaine C. Allard, Val des Monts (CA); Leonid V. Savitch, Kanata (CA); Rajagopal Subramaniam, Ottawa (CA)

(21) Appl. No.: 12/151,046

(22) Filed: May 1, 2008

Related U.S. Application Data
(60) Provisional application No. 60/915,294, filed on May 1, 2007.

(51) Int. Cl. A01H 5/00; C12N 15/82; C12N 15/63; A01H 1/00; C12N 5/04; C07H 21/04

(52) U.S. Cl. 800/279; 536/23.1; 800/301; 435/320.1; 435/419; 435/412; 435/414; 435/417; 435/416; 800/278; 800/298

(57) ABSTRACT

The present invention provides, in part, GLK1 nucleic acid molecules and polypeptides that can be used to confer resistance to a pathogen in a plant. The present invention also provides methods of detecting disease resistance genes and plants.

### FIELD OF INVENTION

The present invention relates to expressing GLK in plants. More specifically, the invention provides GLK1 molecules for conferring disease resistance to plants, increasing nitrogen status in plants, or both.

### BACKGROUND OF THE INVENTION

The economic impact of plant pathogens in relation to agriculture and horticulture is considerable. For example, Fusarium Head Blight (FHB) is a major disease of concern for Canadian cereals.

Global gene expression analyses suggest that induced disease resistance can result from pathogen or pathogen derived molecule-induced reprogramming of gene networks (Li et al, 2006; Bae et al, 2006). The role of transcription factors in the modulation of these networks has been suggested for WRKY domain containing trans-factors (Li et al, 2006) and in the slh1 mutation (Naoutoshi et al, 2005).

Non-host resistance is the term used to describe the phenomenon where all members of a plant species exhibit resistance to all members of a given pathogen species. Non-host resistance is thought to be multigenic; therefore the inactivation of a single gene may not be sufficient to render a plant susceptible to a pathogen. Non-host resistance has been studied in salicylate hydroxylase (NahG) over-expressing plants, which convert salicylic acid to catechol and are defective in non-host response to pathogen. NahG plants expresses the salicylic acid degrading enzyme salicylate hydroxylase, resulting in indirect and side effects in plant defense (Heck et al, 2003; van Wees and Glazebrook 2003). Furthermore, it is known that part of non-host resistance is derived from “priming” of the plant to produce a readiness to infection.

The maize Golden2-like or GLK genes encode proteins belonging to a class of GARP domain of transcriptional activators (Hall, L. N. et al., 1998, Plant Cell 10:925-936; Fitter, W., et al., 2002, The Plant Journal 31: 713-727; Rossini, D. L., et al., 2001, Plant Cell 13: 1231-1244; US 2007/0022495) There are 56 predicted members of the GARP domain containing transcriptional activators in Arabidopsis (Riechmann, J. L., et al, 2000, Science 290: 2105-2110; which is incorporated herein by reference) and are involved in plant specific processes. These include the G2 or GLK proteins that are involved in chloroplast development (Cribb, L. et al., 2001, Genetics 159:787-797; Yasumura, Y., et al., 2005, Plant Cell 17: 1894-1907), the Arabidopsis type-B phosphorelay response regulators (ARR family) that mediate cytokinin signaling (Sakai, H., et al. Science 294:1519-1521; Hosoda, K., 2002, Plant Cell 14: 2015-2029; Mason, M. G., et al., 2005, Plant Cell 17:3007-3018), PSR1 proteins involved in phosphorus metabolism (Wykoff, D. D., et al., 1999, Proc. Natl. Acad. Sci. USA. 96:15336-15341), KANADI proteins in regulation of organ polarity (Kerstetter, R. A., et al., 2001, Nature 411:706-709) and b-ZIP G-box binding factors (Tamai, H., et al., 2002, Plant Cell Physiol. 43:99-107). Recently, increased accumulations of GLK1, 2 transcripts have been observed in constitutively frost tolerant transgenic Brassica napus overexpressing DREB1/CBFs, suggesting that it may play a role in temperature stress response and photosynthetic adaptation (Savitch, L. V., et al., 2005, Plant Cell Physiol. 46:1525-1539).

### SUMMARY OF THE INVENTION

The present invention relates to expressing GLK in plants. More specifically, the invention provides GLK1 molecules for conferring disease resistance to plants, increasing nitrogen status in plants, or both.

The present invention provides a method of conferring resistance to a pathogen in a plant, the method comprising:

i) introducing a nucleic acid molecule encoding a GLK1 polypeptide or fragment or variant thereof into a plant cell to produce a transgenic plant cell, such that the nucleic acid molecule is positioned for expression in the transgenic plant cell; and

ii) growing a transgenic plant from the transgenic plant cell,

wherein the GLK1 polypeptide or fragment or variant thereof is over-expressed relative to wild type GLK1 at least 2 fold, whereby the 2 fold over-expression is sufficient to confer resistance to the pathogen in the plant.

The GLK1 may comprises the nucleic acid sequence set forth in SEQ ID NO: 1, a nucleic acid that exhibits an identity of from about 70 to about 100% with SEQ ID NO:1, or a nucleic acid molecule that hybridizes under conditions of high stringency to the nucleic acid sequence set forth in SEQ ID NO: 1. Furthermore, the GLK1 polypeptide may comprise an amino acid sequence substantially identical to the amino acid sequence set forth in SEQ ID NO: 2. Furthermore, the plant may be selected from the group consisting of canola, Brassica spp., maize, tobacco, alfalfa, potato, ginseng, pea, oat, rice, soybean, wheat, barley, sunflower, and cotton. The pathogen may be selected from the group consisting of Fusarium graminearum, Sclerotinia sclerotium, Puccinia graminis, Puccinia triticinia, Puccinia recondite, and Phytopthora infestans.

The present invention also provides a pathogen resistant plant comprising a GLK1 nucleic acid molecule that encodes a GLK1 polypeptide, wherein expression of the GLK1 produces at least 2 fold greater GLK1 polypeptide relative to a wild-type plant.

The present invention also provides a genetic construct comprising a regulatory region operably linked to a nucleic acid molecule encoding a GLK1 polypeptide or fragment or variant thereof. The nucleic acid molecule may comprise a nucleic acid sequence as set forth in SEQ ID NO: 1, a nucleotide sequence that exhibits from about 70% to about 100% sequence identity with the sequence of SEQ ID NO:1, or a nucleotide sequence that hybridizes to the nucleic acid sequence set forth in SEQ ID NO: 1 under conditions of high stringency. Furthermore, the GLK1 polypeptide may comprise an amino acid sequence set forth in SEQ ID NO: 2. Also provides is a vector comprising the genetic construct as just described. The vector may be pHS723-GLK1.

The present invention also pertains to a host cell, a transgenic plant, or a transgenic plant seed, comprising the genetic construct as described above. The host cell, transgenic plant, or a transgenic seed, may be selected from the group consisting of canola, Brassica spp., maize, tobacco, alfalfa, potato, ginseng, pea, oat, rice, soybean, wheat, barley, sunflower, and cotton. Furthermore, the transgenic plant is characterized as being resistant to a pathogen. The pathogen may be selected from the group consisting of Fusarium graminearum, Sclerotinia sclerotium, Puccinia graminis, Puccinia triticinia, Puccinia recondite, and Phytopthora infestans.

The present invention also provides a method of detecting a candidate pathogen-resistance gene in a plant, comprising, comparing the gene expression profile of a transgenic plant over-expressing a nucleic acid molecule encoding a GLK1 polypeptide or fragment or variant thereof, with the gene expression profile of a wild-type plant; and determining the genes over-expressed in the transgenic plant compared to the wild-type plant, wherein one or more than one of the genes over-expressed in the transgenic plant are candidate pathogen-resistance genes. The present invention also provides a method as just described wherein the GLK1 polypeptide is selected from the group consisting of an Arabidopsis GLK1, a wheat GLK1 polypeptide, a maize GLK1 polypeptide, a rice GLK1 polypeptide, and a soy bean GLK1 polypeptide.

The present invention also pertains to a method of detecting a plant exhibiting resistance to a pathogen, the method comprising:

i) determining the expression levels of a GLK1 nucleic acid molecule or polypeptide or fragment or variant thereof in a test plant;

ii) determining the expression levels of the GLK1 nucleic acid molecule or polypeptide or fragment or variant thereof in a control plant; and

iii) comparing the expression levels in the test plant to the expression levels in the control plant,

wherein the over-expression of the GLK1 nucleic acid molecule or polypeptide or fragment or variant thereof in the test plant compared to the control plant is indicative of the test plant being a plant exhibiting resistance to a pathogen. The test plant may be selected from the group consisting of Arabidopsis, canola, Brassica spp., maize, tobacco, alfalfa, potato, ginseng, pea, oat, rice, soybean, wheat, barley, sunflower, and cotton.

Furthermore the present invention includes a method of detecting a plant exhibiting resistance to a pathogen, the method comprising:

i) determining the expression levels of one or more of the nucleic acid molecule or polypeptide set out in Table 1 or in FIG. 3, or a fragment or variant thereof, in a test plant;

ii) determining the expression levels of the one or more of the nucleic acid molecule or polypeptide set out in Table 1 or in FIG. 3, or a fragment or variant thereof, in a control plant; and

iii) comparing the expression levels in the test plant to the expression levels in the control plant,

wherein the over-expression of the one or more of the nucleic acid molecule or polypeptide set out in Table 1 or in FIG. 3, or a fragment or variant thereof, in the test plant compared to the control plant is indicative of the test plant being a plant exhibiting resistance to a pathogen. The test plant is selected from the group consisting of Arabidopsis, canola, Brassica spp., maize, tobacco, alfalfa, potato, ginseng, pea, oat, rice, soybean, wheat, barley, sunflower, and cotton.

Also provides is a method of detecting a plant exhibiting resistance to a pathogen, the method comprising:

i) determining the expression levels of a At1 g77960, At2g41050, or both At1g77960, At2g41050, nucleic acid molecule or polypeptide or fragment or variant thereof in a test plant;

ii) determining the expression levels of the At1 g77960, At2g41050, or both At1g77960, At2g41050, nucleic acid molecule or polypeptide or fragment or variant thereof in a control plant; and

iii) comparing the expression levels in the test plant to the expression levels in the control plant,

wherein the over-expression of the At1g77960, At2g41050, or both At1g77960, At2g41050, nucleic acid molecule or polypeptide or fragment or variant thereof in the test plant compared to the control plant is indicative of the test plant being a plant exhibiting resistance to a pathogen. The test plant is selected from the group consisting of Arabidopsis, canola, Brassica spp., maize, tobacco, alfalfa, potato, ginseng, pea, oat, rice, soybean, wheat, barley, sunflower, and cotton.

The present invention also provides a method of increasing the nitrogen status in a plant, the method comprising:

i) introducing a nucleic acid molecule encoding a GLK1 polypeptide or fragment or variant thereof into a plant cell to produce a transgenic plant cell, such that the nucleic acid molecule is positioned for expression in the transgenic plant cell; and

ii) growing a transgenic plant from the transgenic plant cell,

wherein the GLK1 polypeptide or fragment or variant thereof is over-expressed relative to wild type GLK1 at least 2 fold, whereby the 2 fold over-expression is sufficient to confer resistance to the pathogen in the plant.

The GLK1 may comprises the nucleic acid sequence set forth in SEQ ID NO: 1, a nucleic acid that exhibits an identity of from about 70 to about 100% with SEQ ID NO:1, or a nucleic acid molecule that hybridizes under conditions of high stringency to the nucleic acid sequence set forth in SEQ ID NO: 1. Furthermore, the GLK1 polypeptide may comprise an amino acid sequence substantially identical to the amino acid sequence set forth in SEQ ID NO: 2. Furthermore, the plant may be selected from the group consisting of canola, Brassica spp., maize, tobacco, alfalfa, potato, ginseng, pea, oat, rice, soybean, wheat, barley, sunflower, and cotton.

The present invention also provides a plant with increased nitrogen status, the plant comprising a GLK1 nucleic acid molecule that encodes a GLK1 polypeptide, wherein expression of the GLK1 produces at least 2 fold greater GLK1 polypeptide relative to a wild-type plant.

The present invention also provides a plant, or a plant cell, that is characterized as having increase nitrogen status. The plant with increased nitrogen status comprising a GLK1 nucleic acid molecule that encodes a GLK1 polypeptide, wherein expression of the GLK1 produces at least 2 fold greater GLK1 polypeptide relative to a wild-type plant. The increase in nitrogen status determined by comparing the plant comprising the GLK1 nucleic acid molecule with a plant that does not express the GLK1 nucleic acid molecule and determining the levels of expression of ASN1, the colour of the leaves, or both determining the levels of expression of ASN1 and the colour of the leaves. Wherein an increase in the expression of ASN1, a darker green leaf, or both determining the levels of expression of ASN1 and the colour of the leaves, when compared to the plant that does not express GLK1, indicates an increase in nitrogen status.

The present invention provides a microarray comprising one or more of the nucleic acid molecule or polypeptide set out in Table 1 or in FIG. 3, or a fragment or variant thereof.

The target genes of GLK over-expression are described herein. The “regulon” of GLK1 was determined by over-expression of Arabidopsis GLK1 (AtGLK1) in Arabidopsis to encode and highly overexpress pathogen and pest related defense proteins (Table 1, FIG. 4). Arabidopsis AtGLK1 OE plants showed a phenotype with enhanced resistance to infection by the pathogen Fusarium graminearum (FIG. 3).

Affymetrix microarray analyses indicated that over-expression of wheat GLK1 (TaGLK1 Accession No. ABL10089) in Arabidopsis shows the regulation of similar genes as in AtGLK1 OE in Arabidopsis. These analyses indicate that TaGLK1 OE is capable of reprogramming identical networks of gene expression as that observed with AtGLK1 OE.

Transgenic wheat (Triticum aestivum cv Fielder) over-expressing wheat TaGLK1 also showed enhanced resistance to the spread of lesions in wheat leaves inoculated with F. graminearum and enhanced resistance in the development of Fusarium Head Blight (FHB) symptoms in developing wheat heads.

As described herein, overexpression of AtGLK1 and TaGLK1 in Arabidopsis also resulted in the upregulation of the glutamine dependent asparagine synthetase1 ASN1 transcripts. The over-expression of AtGLK1 in maize cv. Hii2 produced transgenic maize that had darker green leaves when grown under midwinter (early December, Latitude 46N) greenhouse conditions suggestive of enhanced nitrogen utilization. Over-expression of AtGLK1, TaGLK1,1 or a combination thereof, presents an opportunity to modify nitrogen status in crop plants.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 show various sequences and vectors. FIG. 1A shows the nucleotide of Arabidopsis GLK1 (SEQ ID NO: 1). FIG. 1B shows the amino acid of GLK1 (SEQ ID NO: 2); FIG. 1C shows a schematic diagram of pHS723-70S-AtGLK1-noster, a vector comprising Arabidopsis GLK1; FIG. 1D shows an identity comparison of several known GLK1 amino acid sequences. From top to bottom: Arabidopsis GLK1 polypeptide (accession No. AAK20120; SEQ ID NO: 2), Triticum GLK1 (accession No. ABL10089; SEQ ID NO: 37), Oryza sativa GLK1 (accession No. AAK50393; SEQ ID NO:38), Maize GLK1 (accession No. AAK50392; SEQ ID NO:36), Arabidopsis GLK2 (accession No. AAK20121; SEQ ID NO: 41), Maize GLK2 (SEQ ID NO:39), Oryza sativa GLK2 (accession No. AAK50394; SEQ ID NO:40); FIG. 1E shows the nucleotide sequence of Maize GLK1 (accession No. AF318580.1; SEQ ID NO:33); FIG. 1F shows the nucleotide sequence of Triticum (accession No. EF105406; SEQ ID NO:34); FIG. 1G shows the nucleotide sequence of Oryza sativa (accession No. AF318581.1; SEQ ID NO:35).

FIG. 2 show data obtained from plants over-expressing GLK. FIG. 2A shows Northern blot of GLK over-expressing lines with GLK 1 as probe; FIG. 2B shows RT-PCR of GLK1 over-expressing lines. The left-most lane is a marker lane; WT refers to wild type Arabidopsis plants; and 2-6 and 5-7 refer to GLK1 over-expressing lines; and C) Left panel: Wild Type Arabidopsis; middle panel Arabidopsis over-expressing GLK1 OE line 5-7; right panel Arabidopsis overexpressing GLK1 OE line 2-6. Plants were grown for 24 days under conditions described in Methods.

FIG. 3 shows a comparison of susceptibility to Fusarium graminearum infection between leaves of GLK1 OE lines and wild type by examination of colonization and growth of F. graminearum on leaves. Leaves were inoculated as described in Methods and examined for infection after 96 hours. FIG. 3A shows Infection of leaves of Arabidopsis NahG plants. The appearance of hyphae/mycelia as well as conidiophores are easily discernable. FIG. 3B shows Infection of leaves of wild-type plants. The appearance of hyphae/mycelia as well as conidiophores are easily discernable. FIG. 3C shows Infection of leaves of plants over-expressing GLK1 (line 5-7). Note the lack of infection even after 96 hours.

FIG. 4 shows RT-PCR confirmation of a subset of highly regulated genes identified by Affymetrix analyses. Lanes: WT, wild type; A, GLK1 OE line 2-6; B, GLK1 OE line 5-7

FIG. 5 show the nucleotide and amino acid sequences of At1g77960. FIG. 5A shows the nucleotide sequence (SEQ ID NO: 3) of At1g77960. FIG. 5B shows the amino acid (SEQ ID NO: 4) of At1g77960.

FIG. 6 shows a schematic diagram of the wheat GLK1 vector, pAct-TaGLK1-nos.

FIG. 7 shows Phox-bem1p (PB1) and ASN1 transcripts in TaGLK1oe and AtGLK1oe Arabidopsis lines detected using RT-PCR.

FIG. 8 shows overexpression of TaGLK1 in T1 plants of 3 lines of transgenic wheat, determined using RT-PCR.

FIG. 9 shows TaGLK1 oe vs null labeled.

FIG. 10 shows Fusarium Head Blight (FHB) in TaGLK1oe wheat 14 dpi of F. graminearum. Maturing wheat heads at 50% anthesis were spray individually with 5 ml of F. graminearum inoculum containing 50,000 macroconidia spores/ml. The sprayed plants were placed in a Conviron E15 environmental chamber equipped with a fine water mist that is activated at 30 min intervals and set at 25° C., 16 h light at 200 μEm⁻²

FIG. 11 shows ASN1 expression in Arabidopsis plants overexpressing TaGLK1oe and AtGLK1oe, determined using RT-PCR.

FIG. 12 shows B73 and AtGLK1 OE plants.

FIG. 13 shows vectors for transforming various plants. FIG. 13A shows pTF 101 1 vector for transformation of maize, rice and soybeans with At GLK1. FIG. 13B shows the vector pTF101.1-70S-AtGLK1-nos

### DETAILED DESCRIPTION

The present invention relates to expressing GLK in plants. More specifically, the invention provides GLK1 molecules for conferring disease resistance to plants, increasing nitrogen status in plants, or both.

The present invention provides, in part, GLK1 nucleic acid molecules and polypeptides that can be used to confer resistance to a pathogen in a plant, increase nitrogen status in plants, or both confer resistance to a pathogen in a plant and increase nitrogen status in plants.

Also provided are methods of detecting disease resistance genes and plants. The results described herein indicate that overexpression of GLK1 can alter gene expression networks resulting in the upregulation of defense related genes as well as conferring constitutive resistance to colonization by a pathogen in a plant, for example, F. graminearum in Arabidopsis.

The present invention provides a method for a method of conferring resistance to a pathogen in a plant, comprising:

i) providing a plant comprising a nucleic acid molecule encoding a GLK1 polypeptide, the nucleic acid molecule operatively linked to a non-native regulatory region,

ii) growing the plant and expressing the GLK1 polypeptide,

wherein the GLK1 polypeptide is over-expressed from about 2 to about 100 fold relative to GLK1 produced within a wild type plant, and whereby the amount of over-expressed GLK1 is sufficient to confer resistance to the pathogen in the plant.

The present invention also provides a pathogen resistant plant comprising a GLK1 nucleic acid molecule that encodes a GLK1 polypeptide, wherein expression of the GLK1 produces at least 2 fold greater GLK1 polypeptide relative to a wild-type plant.

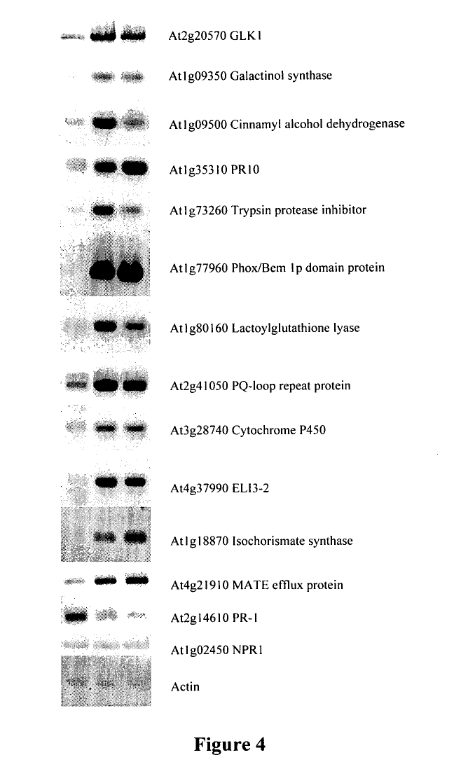

As described herein, it has been observed that plants that over-express GLK1 exhibit resistance to one or more than one pathogen. Without wishing to be bound by theory, this resistance to one or more than one pathogen by a plant in which GLK1 has been over-expressed, may be a result of the direct over-expression of GLK1, or it may arise indirectly from the over-expression of GLK1. For example, over-expression of GLK1 has been shown to induce an assortment of other nucleotide sequences as shown in FIGS. 4, 7 and 11, and Tables 1-3, and these other nucleotide sequences may result in pathogen resistance.

A “pathogen” is an organism, for example a micro-organism, that causes disease. Plant pathogens include, without limitation, Alternaria spp., Ascochyta spp., Aspergillus spp., Botrytis spp., Cercospora spp., Colletotrichum spp., Diplodia spp., Erwinia spp., Erysiphe spp., Fusarium spp. (e.g., Fusarium graminearum), Gaeumanomyces spp., Helminthosporium spp., Macrophomina spp., Magnaporthe spp., Mycosphaerella spp., Nectria spp., Peronospora spp., Phakopsora spp. (e.g., Phakopsora pachyrhizi or Phakopsora meibomiae), Phoma spp., Phymatotrichum spp., Phytopthora spp. (e.g., Phytopthora infestans), Plasmopara spp., Podosphaera spp., Pseudomonas spp., Puccinia spp. (Puccinia graminis, Puccinia recondite, Piccinia triticinia), Puthium spp., Pyrenophora spp., Pyricularia spp., Pythium spp., Rhizoctonia spp., Scerotium spp., Sclerotinia spp. (e.g., Sclerotinia sclerotium), Septoria, Thielaviopsis spp., Uncinula spp., Venturia spp., Verticillium spp., or Xanthomonas spp., etc. and Phytopthora infestans.

Plants that may be infected by one or more of such pathogens include, without limitation, Arabidopsis, canola, Brassica spp., maize, tobacco, alfalfa, potato, tomato ginseng, pea, oat, rice, soybean, wheat, barley, sunflower, cotton, horticultural plants, or other plant.

The present invention also provides a method of increasing the nitrogen status in a plant, the method comprising:

i) introducing a nucleic acid molecule encoding a GLK1 polypeptide or fragment or variant thereof into a plant cell to produce a transgenic plant cell, such that the nucleic acid molecule is positioned for expression in the transgenic plant cell; and

ii) growing a transgenic plant from the transgenic plant cell,

wherein the GLK1 polypeptide or fragment or variant thereof is over-expressed relative to wild type GLK1 at least 2 fold, whereby the 2 fold over-expression is sufficient to confer resistance to the pathogen in the plant.

The present invention also provides a plant, or a plant cell, that is characterized as having increase nitrogen status. The plant with increased nitrogen status comprising a GLK1 nucleic acid molecule that encodes a GLK1 polypeptide, wherein expression of the GLK1 produces at least 2 fold greater GLK1 polypeptide relative to a wild-type plant. The increase in nitrogen status determined by comparing the plant comprising the GLK1 nucleic acid molecule with a plant that does not express the GLK1 nucleic acid molecule and determining the levels of expression of ASN1, the colour of the leaves, or both determining the levels of expression of ASN1 and the colour of the leaves. Wherein an increase in the expression of ASN1, a darker green leaf, or both determining the levels of expression of ASN1 and the colour of the leaves, when compared to the plant that does not express GLK1, indicates an increase in nitrogen status.

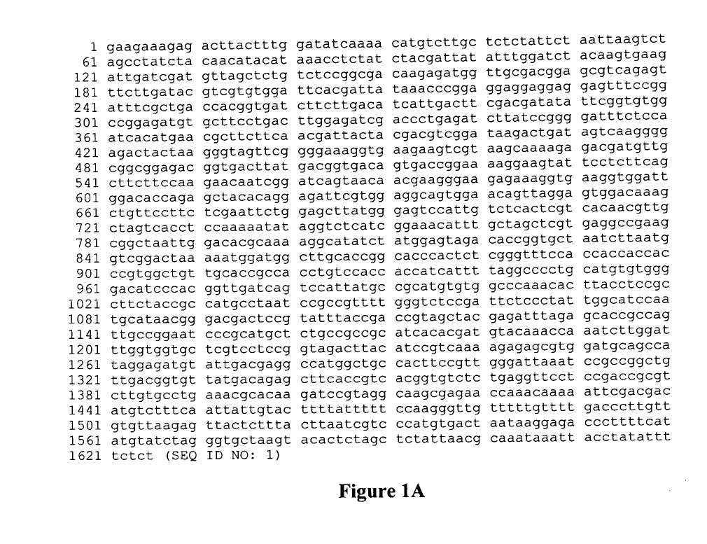

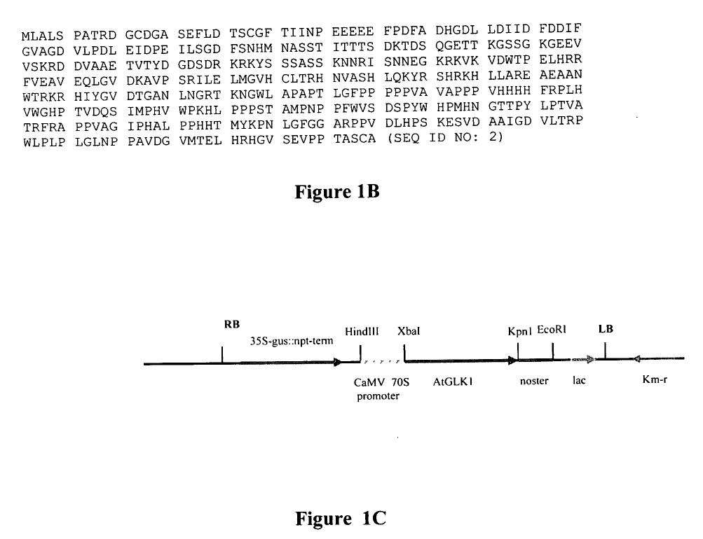

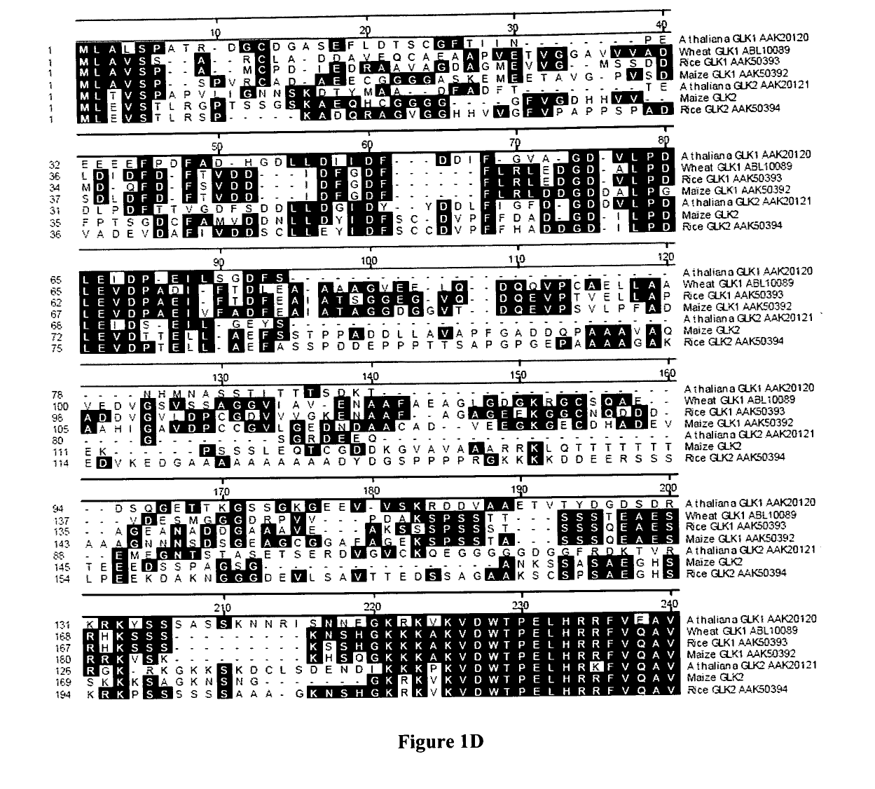

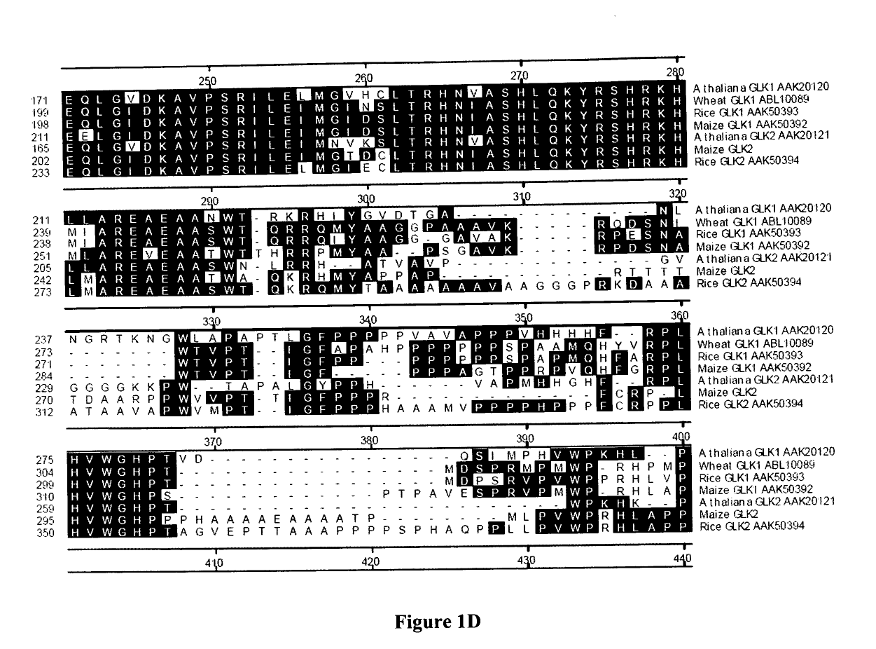

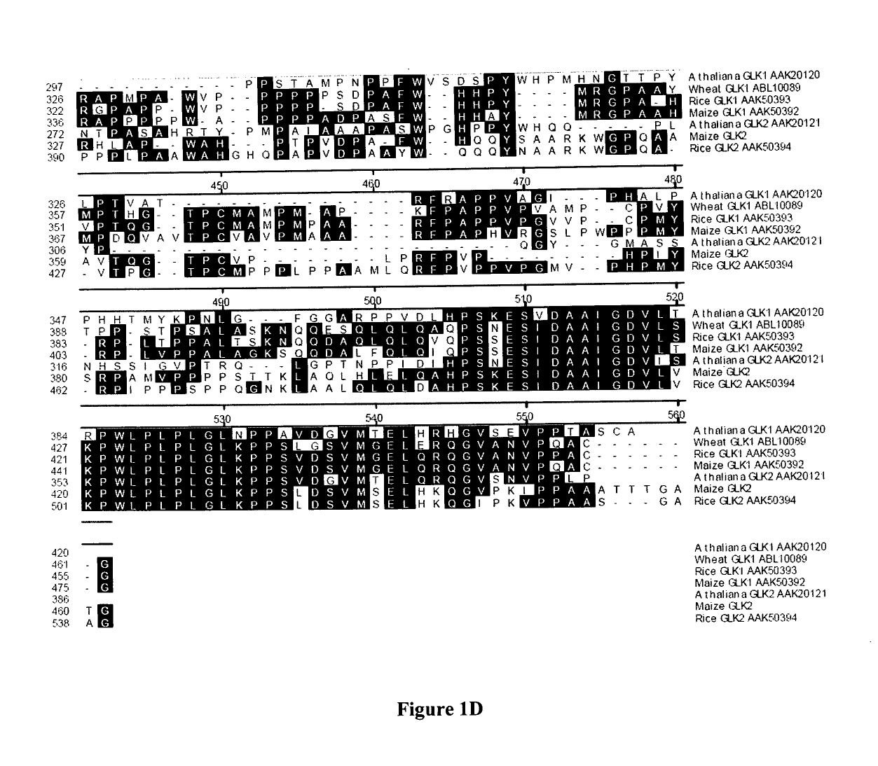

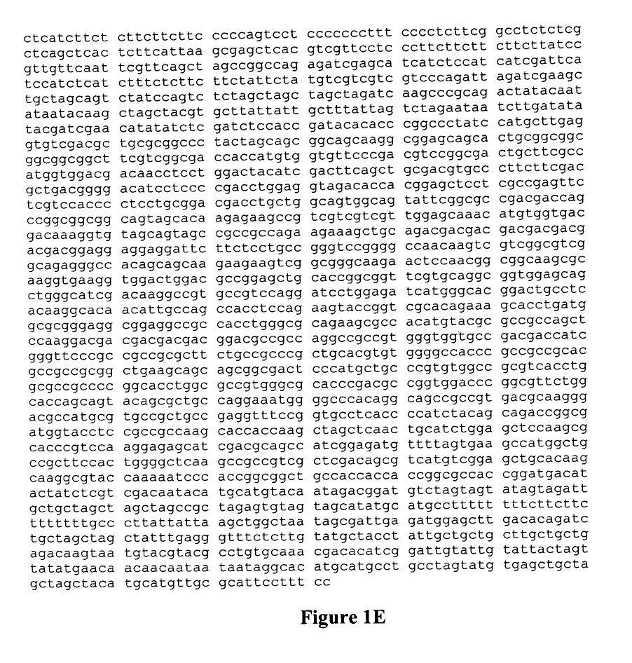

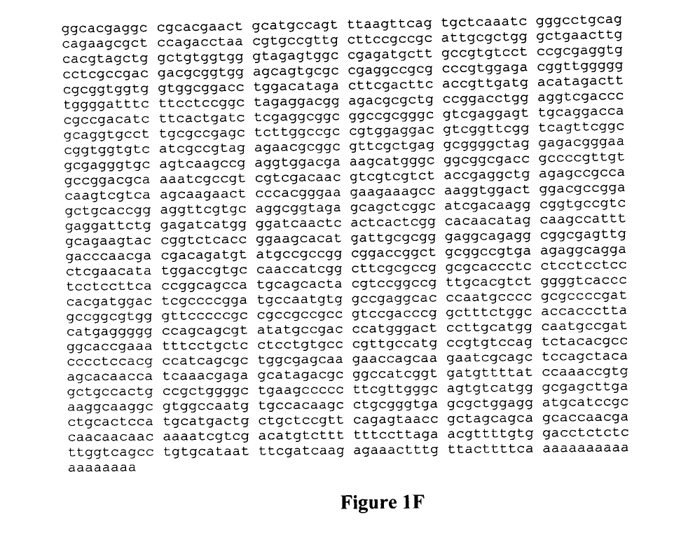

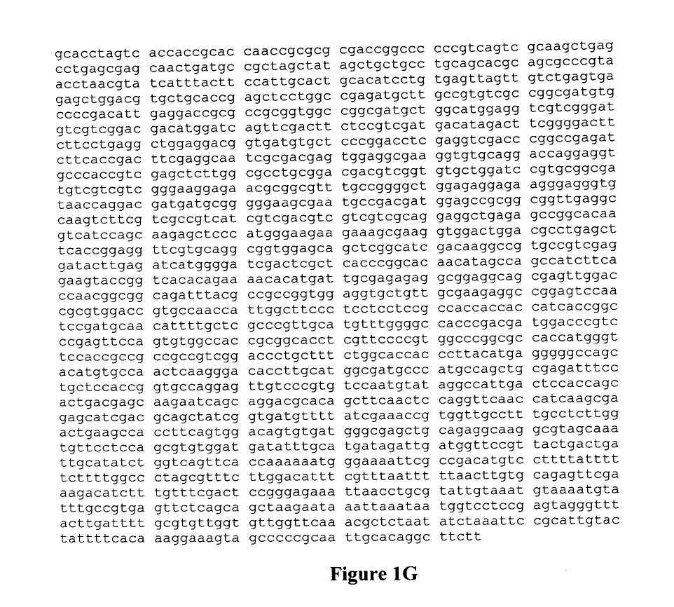

A GLK1 nucleic acid molecule is any nucleic acid molecule that encodes a GLK1 polypeptide as described herein, or a fragment or variant thereof, providing the fragment or variant thereof when expressed in a plant results in resistance to a pathogen when compared to the same plant when the fragment or variant is not expressed. Non-limiting examples of several GLK1 polypeptide sequences may be found in FIG. 1 and are known in the art, for example but not limited to Rossini L. et al. (2001, Plant Cell 13:1231-1244; which is incorporated herein by reference). Accordingly, a GLK1 polypeptide includes an Arabidopsis GLK1 polypeptide (accession No. AAK20120; SEQ ID NO: 2), Triticum GLK (accession No. ABL10089; SEQ ID NO: 37), Maize GLK1 (accession No. AAK50392; SEQ ID NO:36), Oryza saliva (accession No. AAK50393; SEQ ID NO:38), or a fragment or variant thereof, that is capable of conferring resistance to a pathogen in a plant. A GLK1 nucleic acid molecule includes any nucleotide sequence that may encode the amino acid sequences described above, for example, but not limited to SEQ ID NO's: 2, 36, 37 and 38, and include, but are not limited to Arabidopsis GLK1 (NCBI/GenBank accession No. NM 127617.2 or AY028367; SEQ ID NO: 1), Maize GLK1 (accession No. AF318580.1; SEQ ID NO:33), Triticum (accession No. EF105406 or ABL10089; SEQ ID NO:34), Oryza sativa (accession No. AF318581.1; SEQ ID NO:35).

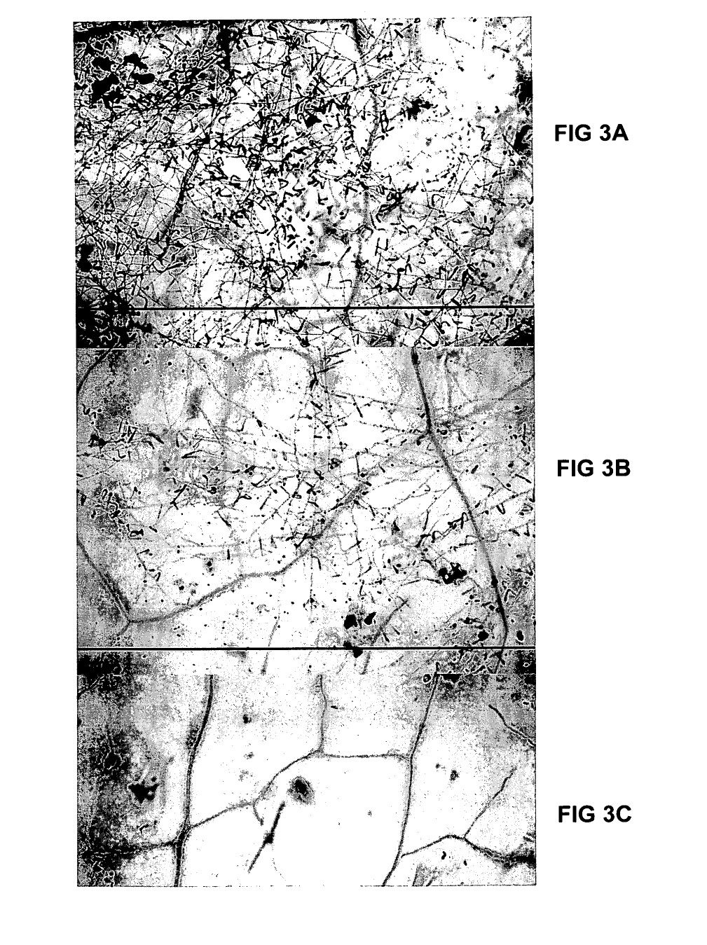

A “pathogen-resistant”, “pathogen resistance”, “disease-resistant”, or “disease resistance” nucleic acid molecule or polypeptide is any nucleic acid molecule or polypeptide, or fragment or variant thereof, that is capable of conferring resistance to a pathogen or a disease in a plant. Pathogen, or disease resistance may be determined by observation of a unit plant surface area, for example as shown in FIGS. 3A-C, where pathogen infestation of a wild-type plant is compared to a plant that is expressing the nucleotide of the present invention that results in pathogen resistance. Pathogen resistance may also be determined quantitatively by measuring the degree of pathogen infestation for a given area, for example, the number or hyphae per unit plant surface area, for example, a leaf surface area, the length of hyphae per unit plant surface area, the number or size of necrotic lesions or other pathogenic factor, per unit plant surface area, and compare the values between a wild-type plant and a plant expressing the nucleic acid sequence of the present invention. A plant that exhibits a reduction of the number of hyphae per unit plant surface area, the length of hyphae per unit plant surface area, the number or size of necrotic lesions or other pathogenic factor, per unit plant surface area, of from about 2% to about 100% or any amount therebetween, is considered pathogen resistant. For example a plant that exhibits a reduction of from about 5% to about 25% or any amount therebetween, 10% to about 75% or any amount therebetween, 25%-75% or any amount therebetween, or a reduction of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100%, in the amount of pathogen infestation for a given area, for example, the number or hyphae per unit plant surface area (e.g. leaf surface area), the length of hyphae per unit plant surface area, the number or size of necrotic lesions or other pathogenic factor, per unit plant surface area is considered pathogen resistant. One or more than one nucleic acid molecule encoding one or more than one polypeptide that results in pathogen resistance in a plant may include a nucleic acid sequence encoding GLK1 as described herein (for example SEQ ID NO:1, or a fragment or variant thereof), the GLK1 polypeptide (for example SEQ ID NO:2, or a fragment or variant thereof), or a nucleic one or polypeptide, that is induced as a result of the over-expression of GLK1 in a plant, for example, one or more than one of the molecules described in Tables 1, 2, 3 or FIGS. 4, 7 and 11, herein.

A nucleic acid molecule or polypeptide that is capable of “conferring resistance” to a pathogen in a plant is a molecule, that when present or over-expressed in a plant, plant cell, plant tissue, or seed, protects that plant, plant cell, plant tissue, or seed from infection by a pathogen i.e., prevents or reduces the disease response of the plant, plant cell, plant tissue, or seed to that pathogen, when compared to the disease response of a plant, plant cell, plant tissue, or seed in which the nucleic acid molecule or polypeptide is absent, under-expressed, or present at “wild-type” levels. In some embodiments, such a nucleic acid molecule or polypeptide confers “non-host resistance.” Such a nucleic acid molecule or polypeptide may include a GLK1 molecule or may include one or more of the molecules described in Table 1 or FIG. 3 herein.

By “operatively linked” it is meant that the particular sequences interact either directly or indirectly to carry out an intended function, such as mediation or modulation of gene expression. The interaction of operatively linked sequences may, for example, be mediated by proteins that interact with the operatively linked sequences. A coding region of interest may also be introduced within a vector along with other sequences, typically heterologous, to produce a chimeric construct.

The present invention contemplates a nucleotide sequence encoding GLK1 or a biologically active fragment or variant thereof, wherein the fragment or variant encodes a polypeptide that confers pathogen resistance when over-expressed. For example, without wishing to be bound by theory, the over-expression of GLK1 may confer pathogen resistance, or it may result in increased expression of one or more than one of a second protein, for example, but not limited to those proteins identified in FIGS. 4, 7, 11, Tables 1-3, or a both FIGS. 4, 7, 11 and Tablet-3. The increased expression of one or more of a second protein may result in pathogen resistance, increased nitrogen status, or both pathogen resistance and increased nitrogen status.

In a preferred embodiment, the nucleotide sequence that is over-expressed in a plant comprises SEQ ID NO:1. However, the nucleotide sequence may also include a variant that comprises between about 70% to 100% sequence identity, or any amount therebetween with SEQ ID NO:1, for example between about 80% to 100% sequence identity, or any amount therebetween with SEQ ID NO:1, between about 85% to 100% sequence identity, or any amount therebetween with SEQ ID NO:1, between about 90% to 100% sequence identity, or any amount therebetween with SEQ ID NO:1, for example, but not limited to about 70%, 72,%, 75%, 78%, 80%, 82%, 85%, 87%, 90%, 91%, 92%, 93%, 94%, 95%, 96%, 97%, 98%, 99% or about 100% similarity, or any amount therebetween, including the sequence identity that may be determined between the GLK1 sequences provided in FIG. 1D. Provided that the fragment or variant thereof when over-expressed in a plant confers pathogen or disease resistance to the plant. For example, as reported in Rossini et al (2001, Plant Cell 13:1231-1244), ZmGLK1 and OsGLK1 exhibit 72.3% sequence identity. The degree of sequence identity between different GLK1 sequences is typically greater than that between GLK1 and GLK2 of the same or different organism (e.g. identity between ZmGLK1 and ZmGLK2 is 49.3%, OsGLK1 and OsGLK2 is 58.0%). It is preferred that the variant or fragment of GLK1 comprise regions of high sequence identity that are known between GLK1 polypeptides (see FIG. 1D) including a GARP domain, comprising a putative DNA binding domain (Rossini et al., 2001, Plant Cell 13:1231-1244; and US 2007/0022495; both of which are incorporated herein by reference) that folds as an HLH in PHD computer predictions, with the amino acid sequence PELHRR (amino acid 230 in FIG. 1D) starting at first helix is about 14 amino acids long and separated from a second helix by about a 22 amino acid loop, with the second helix starts at NI/VASHLQ (amino acid 266 in FIG. 1D).

To determine whether a nucleic acid exhibits sequence identity with the sequences presented herein, the sequence may be aligned and % identity determined manually, or an oligonucleotide alignment algorithm may be used, for example, but not limited to BLAST (GenBank URL: ncbi.nlm.nih.gov/cgi-bin/BLAST/; using default parameters: Program: blastn; Database: nr; Expect 10; filter: default; Alignment: pairwise; Query genetic Codes: Standard (1)), BLAST2 (EMBL URL: embl-heidelberg.de/Services/index.html using default parameters: Matrix BLOSUM62; Filter: default, echofilter: on, Expect: 10, cutoff: default; Strand: both; Descriptions: 50, Alignments: 50), or FASTA (using default parameters). Other similar algorithms may be employed to determine sequence identity between two or more amino acid sequences as would be know to one of skill in the art.

The present invention also includes nucleotide sequences encoding polypeptides having GLK1 activity, that is encoding a polypeptide that confers pathogen resistance when over-expressed in a plant. The nucleotide sequence may encode GLK1 for example, SEQ ID NO:1, or a sequence that hybridizes to SEQ ID NO:1, or a complement of SEQ ID NO:1, under stringent hybridization conditions (see Maniatis et al., in Molecular Cloning (A Laboratory Manual), Cold Spring Harbor Laboratory (1982) p 387 to 389; Ausubel, et al. (eds), 1989, Current Protocols in Molecular Biology, Vol. 1, Green Publishing Associates, Inc., and John Wiley & Sons, Inc., New York, at p. 2.10.3; both of which are incorporated herein by reference). Without wishing to be limiting in any manner, representative examples of stringent hybridization conditions include hybridization in 4×SSC at 65° C. for 8-16 hours, followed by one, two or three washes in 0.1×SSC at 65° C. for an hour, or hybridization in 5×SSC and 50% formamide at 42° C. for 8 to 16 hours, followed by one, two or three washes in about 0.5×SSC to about 0.2×SSC at 65° C. for one hour. However, hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y., which is herein incorporated by reference). Generally, but not wishing to be limiting, stringent conditions are selected to be about 5° C. lower than the thermal melting point for the specific sequence at a defined ionic strength and pH, as can be determined by one of skill in the art.

The GLK1 and other nucleic acid molecules and polypeptides described herein may be isolated and produced using standard recombinant and other techniques as described herein or known in the art e.g. see Maniatis et al., in Molecular Cloning (A Laboratory Manual), Cold Spring Harbor Laboratory (1982) p 387 to 389; Ausubel, et al. (eds), 1989, Current Protocols in Molecular Biology, Vol. 1, Green Publishing Associates, Inc., and John Wiley & Sons, Inc., New York, at p. 2.10.3; both of which are incorporated herein by reference).

By “DNA regulatory region” it is meant a nucleic acid sequence that has the property of controlling the expression of a DNA sequence that is operably linked with the regulatory region. Such regulatory regions may include promoter or enhancer regions, and other regulatory elements recognized by one of skill in the art. By “promoter” it is meant the nucleotide sequences at the 5′ end of a coding region, or fragment thereof that contain all the signals essential for the initiation of transcription and for the regulation of the rate of transcription. The promoters used to exemplify the present invention are constitutive promoters that are known to those of skill in the art. However, if tissue specific expression of the gene is desired, for example seed, or leaf specific expression, then promoters specific to these tissues may also be employed. Furthermore, as would be known to those of skill in the art, inducible promoters may also be used in order to regulate the expression of the gene following the induction of expression by providing the appropriate stimulus for inducing expression. In the absence of an inducer the nucleic acid sequence will not be transcribed. Typically the protein factor, that binds specifically to an inducible promoter to activate transcription, is present in an inactive form which is then directly or indirectly converted to the active form by the inducer.

The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory element may be exposed to an inducer by externally applying the inducer to the cell or plant such as by spraying, watering, heating or similar methods. Inducible elements may be derived from either plant or non-plant genes (e.g. Gatz, C. and Lenk, I. R. P., 1998, Trends Plant Sci. 3, 352-358; which is incorporated by reference). Examples, of potential inducible promoters include, but not limited to, teracycline-inducible promoter (Gatz, C., 1997, Ann. Rev. Plant Physiol. Plant Mol. Biol. 48, 89-108; which is incorporated by reference), steroid inducible promoter (Aoyama, T. and Chua, N. H., 1997, Plant J. 2, 397-404; which is incorporated by reference), for example but not limited to a dexamethasone-induced promoter, and ethanol-inducible promoter (Salter, M. G., et al, 1998, Plant Journal 16, 127-132; Caddick, M. X., et al, 1998, Nature Biotech. 16, 177-180, which are incorporated by reference) cytokinin inducible IB6 and CKI1 genes (Brandstatter, I. and Kieber, J. J., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference). By using an inducible promoter that is operatively linked to the nucleotide sequence of the present invention encoding GLK1, GLK1 may be selectively expressed. This may be desired if the expression of GLK1 leads to reduced plant growth or development, for example if constitutive expression of GLK1 leads to reduced plant growth or delays plant development.

It is also contemplated that the nucleotide sequence of the present invention may also be operatively linked to a wound inducible (Titarenko E., et al., 1997, Plant Physiol. 115:817-826), or pathogenesis related promoter, for example but not limited to a promoter from the gene encoding any of the pathogenesis related proteins PR1-PR15 (van Loon et al., 1999, Phys Mol. Plant. Pathol., 55:85-97; Durrant W. E., Dong. X 2004, Annu. Rev. Phytopatho; 1. 42:185-209, which are incorporated herein by reference), including PRI (e.g. Payne G., et al., 1988, Plant Mol. Biol. 11:89-94; Payne G et al., 1989 Plant Mol. Biol. 12:595-596), or a promoter from PRIa-PRIg (van Loon et al., 1999, Phys Mol. Plant. Pathol., 55:85-97), PR2 (Ward E. R. 1991, Plant Physiol. 96:390-397), PR3 (Payne G., et al. 1990, PNAS 81:98-102), PR4 (Ward E. R., 1991, Plant Cell, 3:1085-1094), PR5 (Payne G., et al., 1998, Plant Mol. Biol. 11:232-234). Additionally, the nucleotide sequence of the present invention may be operatively linked to a promoter encoding a stress-induced protein, resulting from either an abiotic or biotic stress, for example but not limited to a promoter obtained from a jasmonic acid induced gene, for example NPR1 (Kachroo N. A., et al., 2003, Mol. Plant. Microbe Interact. 16:588-599). By using a stress, or would-inducible promoter that is operatively linked to the nucleotide sequence of the present invention encoding GLK1, GLK1 may be induced under conditions that result in its selective expression. This may be desired if the expression of GLK1 leads to reduced plant growth or development.

A constitutive regulatory element directs the expression of a gene throughout the various parts of a plant and continuously throughout plant development. Examples of known constitutive regulatory elements include promoters associated with the CaMV 35S transcript (Odell et al., 1985, Nature, 313: 810-812), the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), tobacco t-CUP promoter (WO/99/67389; U.S. Pat. No. 5,824,872), the HPL promoter (WO 02/50291), and the tobacco translational initiation factor 4A gene (Mandel et al, 1995 Plant Mol. Biol. 29: 995-1004). The term “constitutive” as used herein does not necessarily indicate that a gene under control of the constitutive regulatory element is expressed at the same level in all cell types, but that the gene is expressed in a wide range of cell types even though variation in abundance is often observed.

The chimeric gene construct of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes and the small subunit of the ribulose-1,5-bisphosphate carboxylase (ssRUBISCO) gene. The 3′ untranslated region from the structural gene of the present construct can therefore be used to construct chimeric genes for expression in plants.

The chimeric gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

To aid in identification of transformed plant cells, the constructs of this invention may be further manipulated to include plant selectable markers. Useful selectable markers include enzymes which provide for resistance to an antibiotic such as gentamycin, hygromycin, kanamycin, and the like. Similarly, enzymes providing for production of a compound identifiable by colour change such as GUS (beta-glucuronidase), or luminescence, such as luciferase are useful.

Also considered part of this invention are transgenic plants containing the chimeric gene construct comprising a nucleotide sequence encoding GLK1 as described herein. However, it is to be understood that the nucleotide sequence of the present invention encoding GLK1, may also be combined with a range of regulatory elements for expression within a range of host organisms. Such organisms include, but are not limited to, corn, wheat, barley, oat, tobacco, Brassica, soybean, pea, alfalfa, potato, ginseng, Arabidopsis, horticultural plants, and other plants.

Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. DT. Dennis, D H Turpin, D D Lefebrve, D B Layzell (eds), Addison Wesly, Langmans Ltd. London, pp. 561-579 (1997). For Arabidospsis see Clough and Bent (1998, Plant J. 16, 735-743). The present invention further includes a suitable vector comprising the chimeric gene construct.

As described herein, a network of gene products showed increased transcript accumulation as a result of over-expression of GLK1. The most highly accumulated transcripts of this GLK1 ‘regulon’ encodes genes that are associated with plant defense against mainly biotic stress (Table 1, FIG. 4). Without wishing to be bound by theory, the reprogrammed network may have resulted in the development of a non-host resistant phenotype to a pathogen, for example Fusarium graminearum. This therefore identifies a role for GLK1 other than of chloroplast differentiation as is known in the prior art.

The suite of defense related genes that appear to be associated with the GLK1 regulon include genes that are known to respond to reactive oxygen species (ROS) as well as genes that function in detoxification such as cytochrome p450 (CYP81 family) and MATE efflux proteins. MATE efflux proteins belong to a family of multidrug and toxin extrusion efflux transporters in plants and microbes (Brown, M. H. et al. 1998, Mol. Microbiol. 31:393-395). One member, ALF5, has been shown to be required for resistance to toxins in Arabidopsis (Diener, A. C., et al., 2001, Plant Cell 13:1625-1637). In addition, the eds5 mutation in Arabidopsis encodes a MATE efflux protein that is strongly induced by pathogens, SA application and UV-light exposure (Nawrath, C., et al., 2002, Plant Cell 14: 275-286). The upregulation of MATE efflux protein is consistent with the F. graminearum resistant phenotype observed here as the trichothecene toxin deoxnivalenol (DON) which is produced by the pathogen has been shown to be a factor in infection (Harris, L. J., et al., 2001, Physiol. Mol. P1. Path. 58:173-181).

A pathogen related gene PR10 was also identified to be highly upregulated. PR10 has been observed to be upregulated by infection with pathogens (Riggleman, R. C., 1985, Plant Mol. Biol. 4:81-86) and has been observed to possess antimicrobial activity (C-J. Park, et al., 2004, Plant J. 37:186-198). One of the most highly accumulating transcripts in the GLK1 regulon however, encodes an unknown protein containing a Phox/Bem 1 or PBI domain. The function of this PBI domain containing protein is not known. However, PBI domains are involved in protein-protein interactions and are involved in the activation of NADPH oxidase in responses to ROS as well as suppressing cell death (Simon-Plas, F., et al., 2002, Plant J., 31:137-147; Torres, M. A., et al., 2005, Nature Genetics 37:1130-1134), in MEKK kinase signaling and have also been observed to be upregulated in response to pathogen (Narusaka, Y., et al., 2004, Mol. Plant. Microbe Inter. 17:749-762).

Included in the GLK1 regulon are cinnamyl alcohol dehydrogenase (CAD), and ELI3-2, an aromatic alcohol dehydrogenase. CAD has bee demonstrated to be upregulated in response to pathogen and is implicated in defense lignin biosynthesis, as well as non-host defense in wheat. ELI3-2 encodes an aromatic alcohol dehydrogenase that is upregulated in association with local infection sites. Its expression is has been shown to be dependent on RPM1 expression. GLK1 over-expression also upregulated isochorismate synthase (At1g18870) transcription. Another highly accumulating gene transcript in GLK1 OE is At2g41050, a PQ-loop repeat protein homologous to cystinosin (a lysosomal cystine transporter).

As shown herein, it is possible that GLK1 over expression (OE) may directly transactivate a number of defense related genes. The phenotype of GLK1 OE may be the result of up-regulation, down-regulation, or both up-regulation and down-regulation, of these genes to prepare (prime) the plant for pathogen invasion. Priming is a phenomenon that preconditions the plant to stress. Therefore, one or more than one of the nucleic acid sequences identified in FIG. 3 that exhibit up-regulation, for example of about 2 fold or more, in expression, down-regulation, for example, of 2 fold or more in expression, or both up-regulation and down-regulation, for example, of 2 fold or more, may be used to screen a plant of interest to determine if the plant exhibits pathogen resistance. Example of sequences that may be up or down regulated include those listed in Tables 1-3.

The present invention therefore provides a genetic construct comprising a regulatory region operably linked to a nucleic acid molecule encoding a GLK1 polypeptide or fragment or variant thereof. The nucleic acid molecule may comprise a nucleic acid sequence as set forth in SEQ ID NO: 1, a nucleotide sequence that exhibits from about 70% to about 100% sequence identity with the sequence of SEQ ID NO:1, or a nucleotide sequence that hybridizes to the nucleic acid sequence set forth in SEQ ID NO: 1 under conditions of high stringency. The GLK1 polypeptide may comprise an amino acid sequence set forth in SEQ ID NO: 2. The present invention also pertains to a host cell, a transgenic plant, or a transgenic plant seed, comprising the genetic construct as just described, the transgenic plant is characterized as being resistant to a pathogen, as exhibiting increased nitrogen status, or both resistant to a pathogen and exhibiting increased nitrogen status.

The present invention also provides a method of detecting a candidate pathogen-resistance gene in a plant, comprising, comparing the gene expression profile of a transgenic plant over-expressing a nucleic acid molecule encoding a GLK1 polypeptide or fragment or variant thereof, with the gene expression profile of a wild-type plant; and determining the genes over-expressed in the transgenic plant compared to the wild-type plant, wherein one or more than one of the genes over-expressed in the transgenic plant are candidate pathogen-resistance genes. The present invention also provides a method as just described wherein the GLK1 polypeptide is selected from the group consisting of an Arabidopsis GLK1, a wheat GLK1 polypeptide, a maize GLK1 polypeptide, a rice GLK1 polypeptide, and a soy bean GLK1 polypeptide.

The present invention also pertains to a method of detecting a plant exhibiting resistance to a pathogen, the method comprising:

i) determining the expression levels of a GLK1 nucleic acid molecule or polypeptide or fragment or variant thereof in a test plant;

ii) determining the expression levels of the GLK1 nucleic acid molecule or polypeptide or fragment or variant thereof in a control plant; and

iii) comparing the expression levels in the test plant to the expression levels in the control plant,

wherein the over-expression of the GLK1 nucleic acid molecule or polypeptide or fragment or variant thereof in the test plant compared to the control plant is indicative of the test plant being a plant exhibiting resistance to a pathogen. The test plant may be selected from the group consisting of Arabidopsis, canola, Brassica spp., maize, tobacco, alfalfa, potato, ginseng, pea, oat, rice, soybean, wheat, barley, sunflower, and cotton.

Furthermore the present invention includes a method of detecting a plant exhibiting resistance to a pathogen, the method comprising:

i) determining the expression levels of one or more than one of the nucleic acid molecule or polypeptide set out in Table 1 or in FIG. 3, or a fragment or variant thereof, in a test plant;

ii) determining the expression levels of the one or more of the nucleic acid molecule or polypeptide set out in Table 1 or in FIG. 3, or a fragment or variant thereof, in a control plant; and

iii) comparing the expression levels in the test plant to the expression levels in the control plant,

wherein the over-expression of the one or more of the nucleic acid molecule or polypeptide, reduced expression of the one or more of the nucleic acid molecule or polypeptide, or both over-expression of the one or more of the nucleic acid molecule or polypeptide and reduced expression of the one or more of the nucleic acid molecule or polypeptide, set out in Table 1 or in FIG. 3, or a fragment or variant thereof, in the test plant compared to the control plant is indicative of the test plant being a plant exhibiting resistance to a pathogen. The test plant may be selected from the group consisting of Arabidopsis, canola, Brassica spp., maize, tobacco, alfalfa, potato, ginseng, pea, oat, rice, soybean, wheat, barley, sunflower, and cotton.

The method of detecting a plant (a first plant) that exhibits resistance to a pathogen as described above may comprise detecting a nucleic acid molecule that is expressed at least about 2 fold higher when compared to the expression of the same nucleic acid molecule within a wild-type plant (a second plant). Preferably, the nucleic acid molecule is expressed from about 5 to about 150 fold higher, or any amount therebetween, when compared to the expression of the same nucleic acid molecule within a wild-type plant (a second plant). For example, the nucleic acid molecule may be expressed from about 2, 3, 4, 5, 6, 7, 8, 9, 10, 12, 14, 16, 18, 20, 22, 25, 28, 30, 35, 40, 45, 50, 55, 60, 665, 70, 75, 80, 90, 100, 110, 120, 130, 140, 150 fold higher, or any amount therebetween, when compared to the expression of the same nucleic acid molecule within a wild-type plant (a second plant). This difference in expression may be determined using any method that can differentiate between the levels of expression between the first and second plant, for example but not limited to northern analysis, dot blot analysis, quantitative PCR, a microarray, or other method as would be known to one of skill in the art. Examples of nucleic acids that may be used within this method include those that encode, but are not limited to one or more than one of the following: a putative Mn superoxide dismutase (At3g56350), a glycosyl hydrolase family 1 protein (At3g60140), a Phox/Bem1p domain protein (At1g77960), proline-rich extensin-like family protein (At5g49080), a Bet v I allergen family protein (At1g35310), a trypsin, protease inhibitor family protein (At1g73260), golden2-like transcription factor (GLK1; At2g20570), cinnamyl-alcohol dehydrogenase family protein (At1g09500), a hydroxyproline-rich glycoprotein family protein (At1g77970), a glutamine-dependent asparagine synthetase 1 (At3g47340), a major latex-related protein (At4g23680), a putative mannitol dehydrogenase (ELI3-2; At4g37990), a glycosyl hydrolase family 1 protein (At3g09260), a lactoylglutathione lyase family protein/glyoxalase I family protein (At1g80160), Lactoylglutathione lyase (At1g80160), Isochorismate synthase (At1g18870), a cystinosin PQ-loop repeat protein (At2g41050), MATE efflux protein (At4g21910), Galactinol synthase (At1g09350), or a combination of any of these proteins. Additional nucleic acid markers may also be used as listed in Table 1. It is also contemplated that the proteins encoded by these markers may be used to determine expression of the encoded proteins listed in Table 1. The levels of protein expression may be determined using standard methods including PAGE, staining and scanning the gel, or antibody based techniques, for example ELISA, or diffusion assays as would be known within the art.

Also provided is a method of detecting a plant exhibiting resistance to a pathogen, the method comprising:

i) determining the expression levels of a At1g77960, At2g41050, or both At1g77960 and At2g41050 nucleic acid molecule or polypeptide or fragment or variant thereof in a test plant;

ii) determining the expression levels of the At1g77960, At2g41050, or both At1g77960 and At2g41050 nucleic acid molecule or polypeptide or fragment or variant thereof in a control plant; and

iii) comparing the expression levels in the test plant to the expression levels in the control plant,

wherein the over-expression of the At1g77960, At2g41050, or both At1g77960 and At2g41050 nucleic acid molecule or polypeptide or fragment or variant thereof in the test plant compared to the control plant is indicative of the test plant being a plant exhibiting resistance to a pathogen. The test plant may be selected from the group consisting of Arabidopsis, canola, Brassica spp., maize, tobacco, alfalfa, potato, ginseng, pea, oat, rice, soybean, wheat, barley, sunflower, and cotton.

Affymetrix microarray analyses indicated that over-expression of wheat GLK1 (TaGLK1 Accession No. ABL10089) in Arabidopsis shows the regulation of similar genes as in AtGLK1 OE in Arabidopsis (Tables 1-3).

As shown in FIG. 7, Phox-bem1p (PB1) and ASN1 transcripts are expressed in TaGLK1oe and AtGLK1oe Arabidopsis lines. This data validates the microarray data presented in Table 3 with the genes At1g77960 (PB1) and At3g47340 (ASN1). Taken together, these analyses indicate that TaGLK1 OE is capable of reprogramming identical networks of gene expression as that observed using AtGLK1 OE.

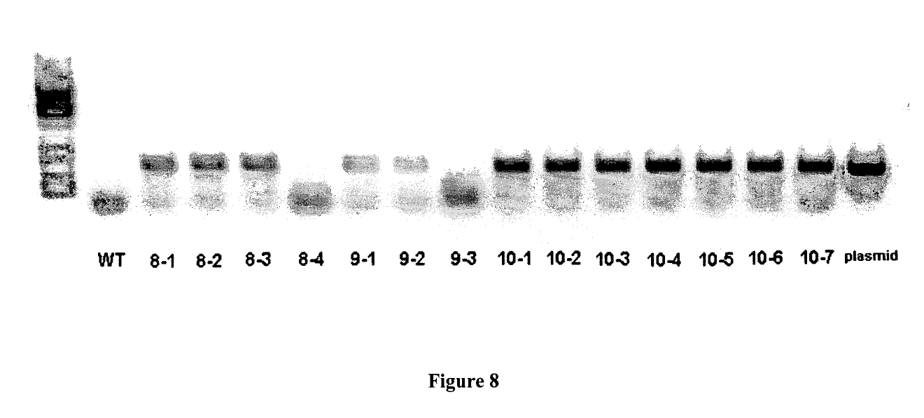

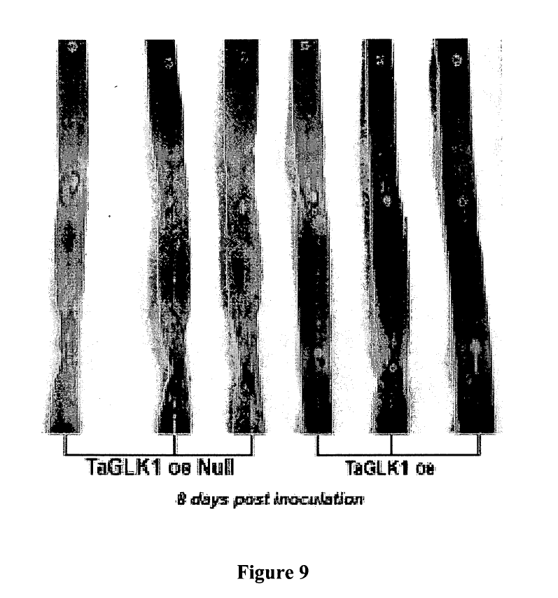

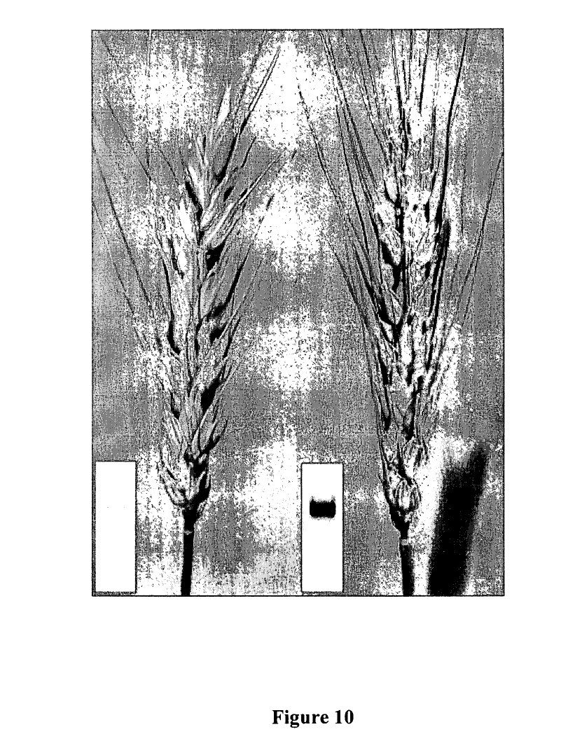

Transgenic wheat (Triticum aestivum cv Fielder) over-expressing wheat TaGLK1 (FIG. 8) shows enhanced resistance to the spread of lesions in wheat leaves inoculated with F. graminearum (FIG. 9) and in the development of Fusarium Head Blight (FHB) symptoms in developing wheat heads (FIG. 10).

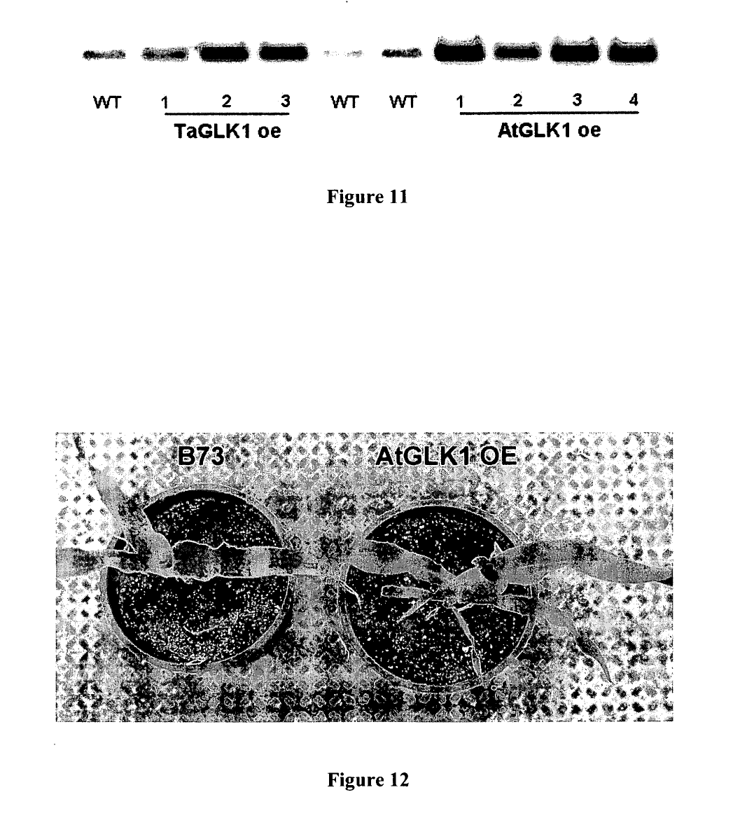

As described herein, overexpression of AtGLK1 and TaGLK1 in Arabidopsis also resulted in the upregulation of the glutamine dependent asparagine synthetase 1 ASN1 transcripts (Table 1-3; FIG. 11). Increases in endogenous levels of ASN1 have been suggested to be associated with higher nitrogen status (Tian et al, 2007, J Biol Chem 282: 18532-18541). Over-expression of bacterial ASN1 is known to enhance the nitrogen status in Arabidopsis and soybean sink tissues (Lam et al, 2003, Plant Physiol 132: 926-935; Wan et al, 2006, Plant Biol (Stuttgart) 8: 271-276), enhanced vegetative growth (Giannino et al, 2007, Euphytica DOI 10.1007/s10681-007-9506-3) and increased synthesis of inulin (Sobolev et al, J Agric Food Chem 55, 10827-10831). The over-expression of AtGLK1 in maize cv. Hii2 produced transgenic maize that had darker green leaves when grown under midwinter (early December, Latitude 46N) greenhouse conditions suggestive of enhanced nitrogen utilization (FIG. 12). This demonstrating that over-expression of At and TaGLK1(s) presents an opportunity to modify nitrogen status in crop plants.

Therefore the present invention also provides a method of increasing the nitrogen status in a plant, the method comprising:

i) introducing a nucleic acid molecule encoding a GLK1 polypeptide or fragment or variant thereof into a plant cell to produce a transgenic plant cell, such that the nucleic acid molecule is positioned for expression in the transgenic plant cell; and

ii) growing a transgenic plant from the transgenic plant cell,

wherein the GLK1 polypeptide or fragment or variant thereof is over-expressed relative to wild type GLK1 at least 2 fold, whereby the 2 fold over-expression is sufficient to confer resistance to the pathogen in the plant.

The present invention also provides a plant, or a plant cell, that is characterized as having increase nitrogen status. The plant with increased nitrogen status comprising a GLK1 nucleic acid molecule that encodes a GLK1 polypeptide, wherein expression of the GLK1 produces at least 2 fold greater GLK1 polypeptide relative to a wild-type plant. The increase in nitrogen status may be observed by comparing the plant comprising the GLK1 nucleic acid molecule with a plant that does not express the GLK1 nucleic acid molecule and determining the levels of expression of ASN1, the colour of the leaves, or both. Wherein an increase in the expression of ASN1, a darker green leaf, or both, when compared to the plant that does not express GLK1, indicates an increased nitrogen status.

Furthermore, the present invention provides a macroarray, or a microarray comprising, but are not limited to, one or more than one nucleic acid that encode: a putative Mn superoxide dismutase (At3g56350), a glycosyl hydrolase family 1 protein (At3g60140), a Phox/Bem1p domain protein (At1g77960), proline-rich extensin-like family protein (At5g49080), a Bet v I allergen family protein (At1g35310), a trypsin, protease inhibitor family protein (At1g73260), golden2-like transcription factor (GLK1; At2g20570), cinnamyl-alcohol dehydrogenase family protein (At1g09500), a hydroxyproline-rich glycoprotein family protein (At1g77970), a glutamine-dependent asparagine synthetase 1 (At3g47340), a major latex-related protein (At4g23680), a putative mannitol dehydrogenase (ELI3-2; At4g37990), a glycosyl hydrolase family 1 protein (At3g09260), a lactoylglutathione lyase family protein/glyoxalase I family protein (At1g80160), Lactoylglutathione lyase (At1g80160), Isochorismate synthase (At1g18870), a cystinosin PQ-loop repeat protein (At2g41050), MATE efflux protein (At4g21910), Galactinol synthase (At1g09350), or a combination thereof. The microarray may be used to determine if a plant of interest exhibits pathogen resistance.

The present invention also provides a macroarray, or a microarray comprising one or more than one of the nucleic acid molecule or polypeptide set out in Table 1 or in FIG. 3, or a fragment or variant thereof. The microarray may be used to determine if a plant of interest exhibits pathogen resistance.

For macroarrays, the following non-limiting method may be used where nucleic acid fragments (with known sequence information) are spotted onto a membrane, for example a nylon membrane (Hybond N, Amersham Pharmacia) in a way to ensure that the location of each DNA fragment is known. The arrayed DNA are fixed to the membrane and hybridized to a labelled probe as described by Sambrook et al., 1989, Molecular Cloning, 2^{nd} ed., Cold Spring Harbor Laboratory Press. Total RNA is isolated from a test plant (for example, according to the method of Chang et al., Plant Mol. Biol. Rep., 1993, 11:113-116), and Poly A+ mRNA isolated. Labelled cDNA is produced by reverse transcription (for example using SuperScript II Rnase H⁻ reverse transcriptase (GIBCO BRL). The labelled cDNA probes are hybridized to the DNA present in the array and detected using any suitable method, for example, fluorescence, or autoradiography.

Microarrays may be produced using methods known in the art (for example, as described at the following URL: affymetrix [dot] com/technology/tech_probe [dot] html). DNA fragments may also be directly spotted onto glass slides. Labelled probes, for example, fluorescently labelled cDNA probes can be synthesized according to the methods outlined at the following URL: uhnres [dot] utoronto [dot] ca/services/microarray/protocols/Pro_RT [dot] html, using poly A⁺ RNA isolated from the appropriate plant tissue, as described above (in DNA macroarrays), and hybridized to the microarray using standard procedures: uhnres [dot] utoronto [dot] ca/services/microarray/protocols/Pro_hybridization [dot] html. Differentially labeled cDNA probes may be used if desired (eg. cDNA produced from two different sources of RNA labeled with Cyanine 3 and Cyanine 5, respectively). The hybridized slides or arrays are then scanned.

The present invention discloses the following sequences:

| SEQ ID NO: | Name |
|---|---|
| 1 | GLK1 Arabidopsis |
| | nucleic acid |
| 2 | GLK1 Arabidopsis |
| | amino acid |
| 3 | At1g77960 nucleic acid |
| 4 | At1g77960 amino acid |
| 5 | GLK1-5′Xba primer |
| 6 | GLK1-3′Kpn primer |
| 7 | At2g20570 5′ primer |
| 8 | At2g20570 3′ primer |
| 9 | At1g09350 5′ primer |
| 10 | At1g09350 3′ primer |
| 11 | At1g09500 5′ primer |
| 12 | At1g09500 3′ primer |
| 13 | At1g35310 5′ primer |
| 14 | At1g35310 3′ primer |
| 15 | At1g73260 5′ primer |
| 16 | At1g73260 3′ primer |
| 17 | At1g77960 5′ primer |
| 18 | At1g77960 3′ primer |
| 19 | At1g80160 5′ primer |
| 20 | At1g80160 3′ primer |
| 21 | At3g0250 5′ primer |
| 22 | At3g0250 3′ primer |
| 23 | At3g60140 5′ primer |
| 24 | At3g60140 3′ primer |
| 25 | At4g37990 5′ primer |
| 26 | At4g37990 3′ primer |
| 27 | At1g18870 5′ primer |
| 28 | At1g18870 3′ primer |
| 29 | At2g14610 5′ primer |
| 30 | At2g14610 3′ primer |
| 31 | 5′ primer |
| 32 | 3′ primer |
| 33 | GLK1 Maize nucleic acid |
| 34 | GLK1 Wheat nucleic acid |
| 35 | GLK1 Rice nucleic acid |
| 36 | GLK1 Maize amino acid |
| 37 | GLK1 Wheat amino acid |
| 38 | GLK1 Rice amino acid |
| 39 | GLK2 Maize amino acid |
| 40 | GLK2 Rice amino acid |
| 41 | AtGLK2 amino acid |
| 42 | At1g02450 5′ primer |
| 43 | At1g02450 3′ primer |
| 44 | primer to TaGLK1 5′ |
| 45 | primer to TaGLK1 3′ |
| 46 | At2g415050 5′ primer |
| 47 | At2g415050 3′ primer |
| 48 | At3g28740 5′ primer |
| 49 | At3g28740 3′ primer |
| 50 | At1g18870 5′ primer |
| 51 | At1g18870 3′ primer |
| 52 | At4g21910 5′ primer |
| 53 | At4g21910 3′ primer |
| 54 | At3g47340 (ASN1) 5′ |
| | primer |
| 55 | At3g47340 (ASN1) 3′ |
| | primer |
| 56 | ABC10089 (TaGLK1) 5′ |
| | primer |
| 57 | ABC10089 (TaGLK1) 3′ |
| | primer |

The present invention will be further illustrated in the following examples.

### EXAMPLES

### Example 1

### Materials and Methods

Growth of plant material: Arabidopsis thaliana (ecotype Columbia) seeds were germinated in soil in 5 cm pots. Seedlings were grown in Conviron E15 chambers at 22° C., 16 hour days at a light intensity of 150 μEm⁻². Fertilization was carried once weekly with 20:20:20: NPK.

RNA isolation: Leaf (2.5 cm rosette) tissues were collected 4 hours after the onset of light period. One gram quantities of leaf (rosette stage, 3 cm long) tissues were collected and frozen in liquid N₂. Total RNA was isolated using Trizol™, P/N 15596-018 (Invitrogen, Burlington, ON, Canada) according to manufacturer's instructions. RNA quality was assessed by electrophoresis on formaldehyde gels.

Cloning of AtGLK1 (AT2G20570): RT-PCR was performed, using total RNA and Superscript III (Invitrogen, Burlington, ON, Canada). First strand synthesis reactions were carried for 1 ug of the isolated RNA with oligo dT (T₂₀VN) and Superscript III (Invitrogen 18080-044). The following primers:

(SEQ ID NO: 5)
GLK1 5′ Xba1-TATATTTGGATCTAGAAGTGAAG;
(SEQ ID NO: 6)
GLK1 3′ Kpn1-TTGGTTCTCGGGTACCTACGG

were used to amplify the resultant cDNA fragment. The PCR fragment was subcloned into pGEM-t-easy (Promega, Madison Wis.) and transformed into E. coli DH5α and the insert was confirmed by sequencing using m13 forward and reverse primers. The fragment was excised with Xba and Kpn1 (introduced by the GLK1 primers) and ligated into pHS723 already containing the 70S-nos-terminator cassette to create pHS723-GLK1. pHS723 contains a NPTII-GUS fusion to allow confirmation of selected plants with GUS (Nair, R. B., et al., 2000, Plant Physiol. 123:1623-1634).

Overexpression of Arabidopsis with GLK1: pHS723-GLK1 was introduced into Agrobacterium tumafaciens (strain GV3109 pmp90) and the resulting transformant was used to infect Arabidopsis by the floral dip method (Clough, S. J., Bent, A. F., 1998, Plant J. 16:735-743). Seeds from Tₒ plants were collected, sterilized and plated on MS media containing 50 μg/ml kanamycin and transformed plants selected as described (Clough, S. J., Bent, A. F., 1998, Plant J. 16:735-743). Plantlets were transferred to soil and GUS staining was used to further confirm integration of the plasmid. GUS stain was also used as a marker to obtain homozygous T₂ plants with single insertions. All further experiments were carried out with T₃ plants.

Arabidopsis Gene Chip Analyses: Affymetrix Gene Chip hybridization and analyses were carried as per manufacturer's protocols at the McGill University and Genome Quebec Innovation Centre (Montreal), using the Affymetrix Arabidopsis ATH1-121501 Gene Chip representing 24,000 genes. For each replicate, 10 ug of total RNA adjusted to a concentration of 1 μg/ul in DMPC water was used. The data was MAS5.1 mean scale normalized. Microarray analyses were carried out as describe in Savitch et al, (2007, Biochem Biophys Res Comm 359: 234-238). Affymetrix Microarray data were analysed by normalization and T-tests using FlexArray program (see URL: genomequebec [dot] mcgill [dot] ca/FlexArray/).

Validation of the microarray data for selected genes was carried out by RT-PCR as described above with the following primers:

(SEQ ID NO: 7)
At2g20570: 5′-TCGGGGAAAGGTGAAGAAGTCGT,
(SEQ ID NO: 8)
3′-CAGCCATGGCCTCGTCAATACA;
(SEQ ID NO: 9)
At1g09350: 5′-ATGGCACCTGAGATGAACAACAAG,
(SEQ ID NO: 10)
3′-GCAAACATATAAGGTACAAAGAGG;
(SEQ ID NO: 11)
At1g09500: 5′-CAACCACATGCACACTAATTC,
(SEQ ID NO: 12)
3′-CCATTGGCTGAAGGAGTCTCG;
(SEQ ID NO: 13)
At1g35310: 5′-CAACATGCCTCCAAAGCCACTC,
(SEQ ID NO: 14)
3′-ACGCATACAATAACTCTCCCACAC;
(SEQ ID NO: 15)
At1g73260: 5′-CTATCAAGCCGCCTCACCTA,
(SEQ ID NO: 16)
3′-CTCACCGACCCGCCAGTA;
(SEQ ID NO: 17)
At1g77960: 5′-ATCGCCGTTGTTGAGTCCTTTATG,
(SEQ ID NO: 18)
3′-AGTATTTGTGCCCCTTTGTTCAGC;
(SEQ ID NO: 19)
At1g80160: 5′-GATGGCGCGTGGTTGTTT,
(SEQ ID NO: 20)
3′-GGGGAGGCTATCGCAGTTG;
(SEQ ID NO: 21)
At3g02500: 5′-CATCTCTAGTACTGCCTCTTCTCC,
(SEQ ID NO: 22)
3′-TGTGTAACCAATATCCCAAAATG;
(SEQ ID NO: 23)
At3g60140: 5′-TGCCTCGGCGTTTCAGTA,
(SEQ ID NO: 24)
3′-TCTCCGACGACCGATTTCT;
(SEQ ID NO: 25)
At4g37990: 5′-CGCCGCACCGCTCCTCTG,
(SEQ ID NO: 26)
3′-GCGGTACCTAACGTCGGCTTTCTC;
(SEQ ID NO: 27)
At1g18870: 5′-AGTGCCCCAGGTCGAGTTTGATG,
(SEQ ID NO: 28)
3′-TCCCGCCTTTCTTTAGTTGATTGG;
(SEQ ID NO: 29)
At2g14610: 5′-CTCAAGATAGCCCACAAGA,
(SEQ ID NO: 30)
3′-AAGGCCACATATTTTACAT
(SEQ ID NO: 42)
At1g02450: 5′-TACAATTATTCCCTAGAGACCAT,
(SEQ ID NO: 43)
3′-TACAATTATTCCCTAGAGACCAT
(SEQ ID NO: 46)
At2g41050 5′-TATAAACCCGTCGACTCATTCATT
(SEQ ID NO: 47)
3′-GGGCAAGTCATTCATATCTAAGGA
(SEQ ID NO: 48)
At3g28740 5′-CGGCCGCGTAAACTAAACCTA
(SEQ ID NO: 49)
3′-TCCCGGCAAGTATCATAACAAGTA
(SEQ ID NO: 50)
At1g18870 5′-AGTGCCCCAGGTCGAGTTTGATG
(SEQ ID NO: 51)
3′-TCCCGCCTTTCTTTAGTTGATTGG
(SEQ ID NO: 52)
At4g21910 5′-GTTGGCACTACCGGCGATACTTA
(SEQ ID NO: 53)
3′-ACACCCAACCGGAATACCAACAAC
(SEQ ID NO: 54)
At3g47340(ASN1) 5′-GTGGAATACTTGCCGTGTTAGGAT
(SEQ ID NO: 55)
3′-CACGAGCCACCATGAAAGAGTTAT
(SEQ ID NO: 56)
ABL10089(TaGLK1) 5′-CCGCGGGCGTCGAGGAGTTG
(SEQ ID NO: 57)
3′-CGGCGGCATACATCTGTCGTCGTT

Fusarium graminearum Infection Bioassay: Assessment of Susceptibility to Fusarium graminearum:

Fungal challenge of Arabidopsis leaves with F. graminearum strain DOAM233423 was carried out essentially as described by Makandar et al (Makandar, R., et al., 2006, Mol. Plant. Microbe Interact. 19:23-129) with the following modifications. Two centimeter rosette leaves from four to 5 weeks old Arabidopsis seedlings grown as described above were used for fungal inoculation. A fungal inoculum containing fifty-thousand spores per ml was infiltrated into the abaxial surface of leaves with a needle-less syringe. The pathogen-inoculated plants were covered with a clear plastic dome to maintain high humidity. The disease spread was recorded 48 hrs and 96 hrs post inoculation by Trypan-blue (TB) stain. After destaining, the leaves were stored in 60% glycerol and examined for presence of mycelia under the light microscope.

### Example 2

### Overexpression of GLK1

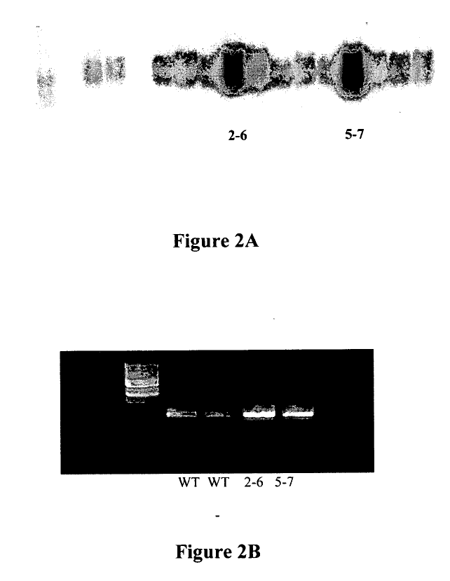

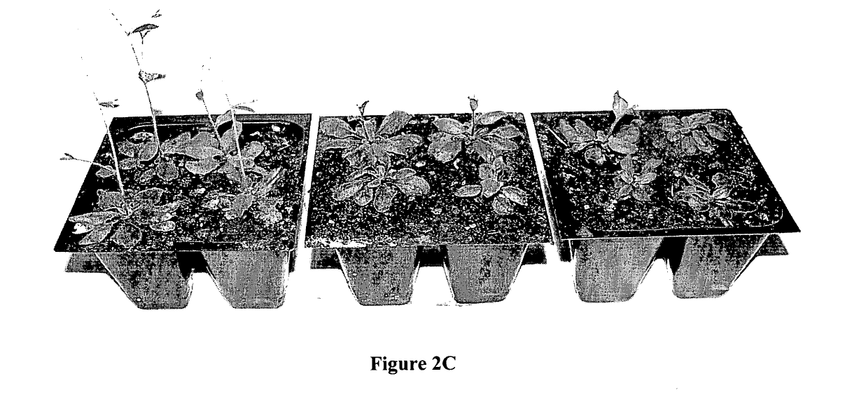

To study the effect of GLK in gene regulation, GLK1 and GLK2 were studied in Arabidopsis. More specifically, Arabidopsis GLK 1 cDNA (SEQ ID NO: 1; FIG. 1A), regulated by the tandem 35S promoter (FIG. 1C), was inserted into the transformation vector pHS723 (Nair et al., 2000) to produce the plasmid pHSGLK1. The plasmid was introduced into Arabidopsis by the floral dip method (Clough and Bent, 1998, Plant J. 16:735-743) and two homozygous independent transgenic lines (2-6 and 5-7) exhibiting increased accumulation of AtGLK1 transcripts i.e., over-expressing AtGLK1 were obtained, as confirmed by Northern blotting using full length AtGLK1 (SEQ ID NO:1) and RT-PCR (FIG. 2A-B) using the following primers:

(SEQ ID NO: 31)
5′ primer-tcggggaaaggtgaagaagtcgt
(SEQ ID NO: 32)
3′ primer-cagccatggcctcgtcaataca

Seedlings (T5) of both GLK1 OE lines were approximately 5 days slower in growth than the wild type under conditions specified in the Methods section (FIG. 2C).

### Example 3

### Analysis of Disease Phenotypes

The ability of the two independent GLK1 over-expressing lines to respond to pathogenesis was studied using the hypersensitive response test and by resistance to Fusarium graminearum infection.

The hypersensitive response was induced by challenging the leaves with Pseudomonas syringae. Both GLK1 over-expressing lines were able to initiate a hypersensitive response much more rapidly than wild type.

The leaves of transgenic Arabidopsis over-expressing GLK1 were also challenged with a virulent strain of F. graminearum conidia (Chen et al, 2006) and compared to both the wild type (WT) and an Arabidopsis line over-expressing the NahG transgene. The ability of the leaves of the GLK1 over-expressing line to inhibit mycelial growth of Fusarium graminearum was dramatic and is illustrated in FIGS. 3A-3C, which show comparisons between NahG (a SA impaired line that has lost its ability to develop SAR and is susceptible to infection (Gaffney, T., et al. 1993, Science 261:754-756), the wild type (WT) and the GLK1 OE (5-7) lines. The responses to infection were followed over a time course. At four days post inoculation, the presence of the highest density of hyphae and conidiophores was observed on the NahG line (FIG. 3A) followed by the WT line (FIG. 3B). No mycelial growth of the fungus was observed on the leaves of the GLK1 OE line (FIG. 3C). The phenotypic results were repeatable and similar results were obtained with the GLK1 OE 2-6 line. The resistance to infection is consistent with the upregulation of defense related genes by GLK1.

### Example 4

### Global Analyses of Gene Expression

Global analyses of the effects of GLK1 over-expression (GLK1 OE) on gene expression were carried out by comparing three biological replicates of GLK OE against three biological replicates of the WT (wild-type) controls using the A. thaliana 25K Affymetrix^{R} gene chip.

Total RNA samples obtained from biological replicates of three wild type, one GLK1 OE line 2-6 and two GLK1 OE line 5-7 leaves (Table 1), and TaGLK 1 OE (Table 2, see Example 6) Arabidopsis plants were used individually for the hybridizations with six Affymetrix Arabidopsis ATH1-121501 Gene Chips. The data was obtained from un-paired analyses of normalized values and represented differences that were statistically significant. The results identified 379 genes of which transcripts encoding one hundred and ninety-four genes were identified to be significantly upregulated and one hundred and forty-six downregulated more than 2 fold (Table 1; “Genes significantly up or down regulated 2-fold in Arabidopsis overexpressing (OE) Arabidopsis GLK1 (AtGLK1). Data analysed using FlexArray for normalization and T-Test”). The most highly accumulated transcripts encode proteins that have been identified in defense responses and include both PR genes as well as genes of enzymatic function implicated in biotic stress responses.

Table 2 identifies genes significantly up or down regulated 2-fold in Arabidopsis overexpressing (OE) wheat GLK1 (TaGLK1, see Example 6; Data analysed using FlexArray for normalization and T-Test)

Table 3 identifies common genes significantly up or down regulated 2-fold in Arabidopsis overexpressing (OE) Arabidopsis GLK1 (AtGLK1) and wheat GLK1 (TaGLK1; Data analysed using FlexArray for normalization and T-Test).

Affymetrix microarray analyses indicated that over-expression of wheat GLK1 (TaGLK1 Accession No. ABL10089) in Arabidopsis shows the regulation of similar genes as in AtGLK1 OE in Arabidopsis (Tables 1-3).

Some of the genes identified by the microarray were confirmed by RT-PCR. The most highly accumulated transcripts, confirmed by RT-PCR, include PB 1 (Phox bem1p domain protein) and ASN I (glutamine-dependent asparagine synthetase 1; FIG. 7). Phox-bem1p (PB1) and ASN1 transcripts are expressed in TaGLK1oe and AtGLK1oe Arabidopsis lines. This data validates the microarray data presented in Table 3 with the genes At1g77960 (PB1) and At3g47340 (ASN1). Taken together, these analyses indicate that TaGLK1 OE is capable of reprogramming identical networks of gene expression as that observed using AtGLK1 OE.

Increases in endogenous levels of ASN1 have been suggested to be associated with higher nitrogen status (Tian et al, 2007, J Biol Chem 282: 18532-18541). Over-expression of bacterial ASN1 is known to enhance the nitrogen status in Arabidopsis and soybean sink tissues (Lam et al, 2003, Plant Physiol 132: 926-935; Wan et al, 2006, Plant Biol (Stuttgart) 8: 271-276), enhanced vegetative growth (Giannino et al, 2007, Euphytica DOI 10.1007/s10681-007-9506-3) and increased synthesis of inulin (Sobolev et al, J Agric Food Chem 55, 10827-10831). The over-expression of AtGLK1 in maize cv. Hii2 produced transgenic maize that had darker green leaves when grown under midwinter (early December, Latitude 46N) greenhouse conditions suggestive of enhanced nitrogen utilization (FIG. 12). This demonstrating that over-expression of At and TaGLK1(s) presents an opportunity to modify nitrogen status in crop plants.

Additional highly accumulated transcripts represented potentially defense-related genes (FIG. 4). PR1, a gene indicative of the activation of the SAR (Uknes, S., et al., 1992, Plant Cell 4: 645-656) was downregulated as shown in both microarray and RT-PCR analyses (FIG. 4).

Microarray analyses of GLK2 over-expressing plants did not show the same profiles in defense related gene expression as observed in GLK1 over-expressing plants.

TABLE 1
Genes significantly up or down regulated 2-fold in Arabidopsis overexpressing
(OE) Arabidopsis GLK1 (AtGLK1). Data analysed using FlexArray for normalization and T-
Test

| | | AtGLK1 |
|---|---|---|
| | | OE Fold |
| Transcript ID | Gene_Title | change |
| At1g77960 | Phox bem1p domain protein | 42.25 |
| At3g47340 | glutamine-dependent asparagine synthetase 1 (ASN1) | 12.29 |
| At2g20570 | golden2-like transcription factor (GLK1) | 11.56 |
| At2g39030 | GCN5-related N-acetyltransferase (GNAT) family protein | 10.94 |
| At4g23680 | major latex protein-related/MLP-related | 9.92 |
| At5g22500 | acyl CoA reductase, putative/male-sterility protein, putative | 8.42 |
| At5g45670 | GDSL-motif lipase/hydrolase family protein | 8.01 |
| At3g55970 | oxidoreductase, 2OG-Fe(II) oxygenase family protein | 8.01 |
| At1g77970 | hydroxyproline-rich glycoprotein family protein | 7.85 |
| At5g37300 | expressed protein | 7.39 |
| At2g37770 | aldo/keto reductase family protein | 7.37 |
| At1g35310 | Bet v I allergen family protein | 6.64 |
| At3g56350 | superoxide dismutase (Mn), putative | 6.47 |
| At2g20670 | expressed protein | 6.28 |
| At5g44530 | subtilase family protein | 5.98 |
| At3g60140 | glycosyl hydrolase family 1 protein | 5.92 |
| At1g09350 | galactinol synthase, putative | 5.82 |
| At1g13650 | expressed protein | 5.66 |
| At3g11480 | S-adenosyl-L-methionine:carboxyl methyltransferase family protein | 5.60 |
| At4g15210 | beta-amylase (BMY1)/1,4-alpha-D-glucan maltohydrolase | 5.40 |
| At3g16460 | jacalin lectin family protein | 5.15 |
| At1g73260 | trypsin and protease inhibitor family protein/Kunitz family | 4.95 |
| At2g23000 | serine carboxypeptidase S10 family protein | 4.84 |
| At2g21220 | auxin-responsive protein, putative | 4.73 |
| At4g22470 | protease inhibitor/seed storage/lipid transfer protein (LTP) family protein | 4.69 |
| At4g37990 | mannitol dehydrogenase, putative (ELI3-2) | 4.69 |
| At1g56650 | myb family transcription factor (MYB75) | 4.67 |
| At5g48490 | protease inhibitor/seed storage/lipid transfer protein (LTP) family protein | 4.49 |
| At1g78170 | expressed protein | 4.47 |
| At1g79460 | ent-kaurene synthase/ent-kaurene synthetase B (KS) (GA2) | 4.39 |
| At2g35290 | expressed protein | 4.39 |
| At1g14250 | nucleoside phosphatase family protein/GDA1/CD39 family protein | 4.37 |
| At3g15720 | glycoside hydrolase family 28 protein/polygalacturonase (pectinase) | 4.31 |
| | family protein | |
| At5g24780 | vegetative storage protein 2 (VSP2) | 4.20 |
| At5g62165 | MADS-box protein (AGL42) | 4.14 |
| At1g09500 | cinnamyl-alcohol dehydrogenase family/CAD family | 4.14 |
| At2g33380 | calcium-binding RD20 protein (RD20) | 4.10 |
| At3g04000 | short-chain dehydrogenase/reductase (SDR) family protein | 3.99 |
| At5g36910 | thionin (THI2.2) | 3.99 |
| At1g52800 | oxidoreductase, 2OG-Fe(II) oxygenase family protein | 3.96 |
| At2g46790 | pseudo-response regulator, putative/timing of CAB expression 1-like | 3.92 |
| | protein, | |
| At2g30790 | photosystem II oxygen-evolving complex 23, putative | 3.86 |
| At5g09730 | glycosyl hydrolase family 3 protein | 3.83 |
| At1g77210 | sugar transporter, putative | 3.79 |
| At2g01890 | purple acid phosphatase, putative | 3.78 |
| At5g42800 | dihydroflavonol 4-reductase (dihydrokaempferol 4-reductase) (DFR) | 3.74 |
| At4g35060 | heavy-metal-associated domain-containing protein/copper chaperone | 3.73 |
| | (CCH)-related | |
| At5g49080 | proline-rich extensin-like family protein | 3.72 |
| At5g35490 | expressed protein (MRU1) | 3.70 |
| At1g10585 | expressed protein | 3.64 |
| At5g23820 | MD-2-related lipid recognition domain-containing protein/ML domain- | 3.58 |
| | containing protein | |
| At1g35290 | thioesterase family protein | 3.55 |
| At1g53885 | senescence-associated protein-related | 3.54 |
| At2g47180 | galactinol synthase, putative | 3.49 |
| At5g59480 | haloacid dehalogenase-like hydrolase family protein | 3.47 |
| At4g09020 | isoamylase, putative/starch debranching enzyme, putative | 3.41 |
| At5g50915 | basic helix-loop-helix (bHLH) family protein | 3.40 |
| At2g15090 | fatty acid elongase, putative | 3.37 |
| At2g42530 | cold-responsive protein/cold-regulated protein (cor15b) | 3.35 |
| At1g80160 | lactoylglutathione lyase family protein/glyoxalase I family protein | 3.35 |
| At2g37900 | proton-dependent oligopeptide transport (POT) family protein | 3.32 |
| At5g59310 | lipid transfer protein 4 (LTP4) | 3.25 |
| At1g17020 | oxidoreductase, 2OG-Fe(II) oxygenase family protein | 3.19 |
| At2g39920 | acid phosphatase class B family protein | 3.18 |
| At4g37770 | 1-aminocyclopropane-1-carboxylate synthase, putative/ACC synthase, | 3.13 |
| | putative | |
| At3g15950 | DNA topoisomerase-related | 3.11 |
| At3g08920 | rhodanese-like domain-containing protein | 3.05 |
| At2g34810 | FAD-binding domain-containing protein | 3.04 |
| At1g49430 | long-chain-fatty-acid--CoA ligase/long-chain acyl-CoA synthetase | 3.03 |
| At3g09260 | glycosyl hydrolase family 1 protein | 2.97 |
| At2g38870 | protease inhibitor, putative | 2.96 |
| At5g50950 | fumarate hydratase, putative/fumarase, putative | 2.94 |
| At1g77580 | myosin heavy chain-related | 2.92 |
| At3g28220 | meprin and TRAF homology domain-containing protein/MATH domain- | 2.92 |
| | containing protein | |
| At1g34040 | alliinase family protein /// alliinase family protein | 2.92 |
| At2g29490 | glutathione S-transferase, putative | 2.91 |
| At1g52690 | late embryogenesis abundant protein, putative/LEA protein, putative | 2.88 |
| At4g12500 | protease inhibitor/seed storage/lipid transfer protein (LTP) family protein | 2.86 |
| At2g46680 | homeobox-leucine zipper protein 7 (HB-7)/HD-ZIP transcription factor 7 | 2.85 |
| At2g21650 | myb family transcription factor | 2.81 |
| At2g38180 | GDSL-motif lipase/hydrolase family protein | 2.81 |
| At3g28740 | cytochrome P450 family protein | 2.81 |
| At2g48020 | sugar transporter, putative | 2.81 |
| At1g73870 | zinc finger (B-box type) family protein | 2.77 |
| At4g12490 | protease inhibitor/seed storage/lipid transfer protein (LTP) family protein | 2.77 |
| At3g13672 | seven in absentia (SINA) family protein | 2.74 |
| At1g18870 | isochorismate synthase, putative/isochorismate mutase, putative | 2.73 |
| At2g42190 | expressed protein | 2.72 |
| At5g67520 | adenylylsulfate kinase, putative | 2.70 |
| At5g24420 | glucosamine/galactosamine-6-phosphate isomerase-related | 2.69 |
| At2g43510 | trypsin inhibitor, putative | 2.69 |
| At1g56170 | transcription factor, putative | 2.67 |
| At1g16530 | LOB domain protein 3/lateral organ boundaries domain protein 3 | 2.67 |
| | (LBD3) | |
| At2g22240 | inositol-3-phosphate synthase isozyme 2/myo-inositol-1-phosphate | 2.66 |
| | synthase 2/MI-1-P synthase 2/IPS 2 | |
| At4g21910 | MATE efflux family protein | 2.66 |
| At5g24160 | squalene monooxygenase 1,2/squalene epoxidase 1,2 (SQP1,2) | 2.65 |
| At3g29810 | phytochelatin synthetase family protein/COBRA cell expansion protein | 2.64 |
| | COBL2 | |
| At1g58290 | glutamyl-tRNA reductase 1/GluTR (HEMA1) | 2.64 |
| At1g64200 | vacuolar ATP synthase subunit E, putative | 2.64 |
| At4g21680 | proton-dependent oligopeptide transport (POT) family protein | 2.63 |
| At4g10850 | nodulin MtN3 family protein | 2.62 |
| At1g69160 | expressed protein | 2.60 |
| At2g36050 | ovate protein-related | 2.60 |
| At5g49360 | glycosyl hydrolase family 3 protein | 2.60 |
| At5g47610 | zinc finger (C3HC4-type RING finger) family protein | 2.60 |
| At3g20100 | cytochrome P450 family protein | 2.60 |
| At1g15010 | expressed protein | 2.60 |
| At5g05600 | oxidoreductase, 2OG-Fe(II) oxygenase family protein | 2.59 |
| At4g19020 | chromomethylase 2 (CMT2) | 2.57 |
| At2g34070 | expressed protein | 2.56 |
| At5g43580 | protease inhibitor, putative | 2.56 |
| At5g05860 | UDP-glucoronosyl/UDP-glucosyl transferase family protein | 2.56 |
| At4g13560 | late embryogenesis abundant domain-containing protein/LEA domain- | 2.55 |
| | containing protein | |
| At1g55960 | expressed protein | 2.54 |
| At4g23600 | coronatine-responsive tyrosine aminotransferase/tyrosine transaminase | 2.53 |
| At2g36080 | DNA-binding protein, putative | 2.52 |
| At5g24150 | squalene monooxygenase 1,1/squalene epoxidase 1,1 (SQP1,1) | 2.52 |
| At1g79160 | expressed protein | 2.52 |
| At1g02220 | no apical meristem (NAM) family protein | 2.50 |
| At4g39510 | cytochrome P450 family protein | 2.50 |
| At3g29575 | expressed protein | 2.50 |
| At5g65020 | annexin 2 (ANN2) | 2.49 |
| At2g30540 | glutaredoxin family protein | 2.48 |
| At5g52570 | beta-carotene hydroxylase, putative | 2.47 |
| At3g02480 | ABA-responsive protein-related | 2.46 |
| At5g02940 | expressed protein | 2.45 |
| At2g02990 | ribonuclease 1 (RNS1) | 2.45 |
| At5g59220 | protein phosphatase 2C, putative/PP2C, putative | 2.45 |
| At1g66760 | MATE efflux family protein | 2.44 |
| At5g17220 | glutathione S-transferase, putative | 2.44 |
| At4g27440 | protochlorophyllide reductase B, chloroplast/PCR B/NADPH- | 2.44 |
| | protochlorophyllide oxidoreductase B (PORB) | |
| At4g01430 | nodulin MtN21 family protein | 2.44 |
| At2g39330 | jacalin lectin family protein | 2.44 |
| At1g11300 | S-locus lectin protein kinase family protein | 2.44 |
| At1g62660 | beta-fructosidase (BFRUCT3)/beta-fructofuranosidase/invertase, | 2.43 |
| | vacuolar | |
| At1g59930 | hypothetical protein | 2.43 |
| At4g17470 | palmitoyl protein thioesterase family protein | 2.43 |
| At3g02380 | zinc finger protein CONSTANS-LIKE 2 (COL2) | 2.43 |
| At5g01520 | zinc finger (C3HC4-type RING finger) family protein | 2.43 |
| At4g33666 | expressed protein | 2.42 |
| At2g25900 | zinc finger (CCCH-type) family protein | 2.42 |
| At1g68190 | zinc finger (B-box type) family protein | 2.41 |
| At4g33150 | lysine-ketoglutarate reductase/saccharopine dehydrogenase bifunctional | 2.41 |
| | enzyme | |
| At3g14770 | nodulin MtN3 family protein | 2.40 |
| At1g02340 | long hypocotyl in far-red 1 (HFR1)/reduced phytochrome signaling | 2.40 |
| | (REP1)/basic helix-loop-helix FBI1 protein (FBI1)/reduced sensitivity | |
| | to far-red light (RSF1)/bHLH protein 26 (BHLH026) (BHLH26) | |
| At5g44050 | MATE efflux family protein | 2.39 |
| At4g19430 | expressed protein | 2.39 |
| At3g06500 | beta-fructofuranosidase, putative/invertase, putative/saccharase, | 2.39 |
| | putative/beta-fructosidase, putative | |
| At5g57340 | expressed protein | 2.38 |
| At1g20190 | expansin, putative (EXP11) | 2.38 |
| At4g37530 | peroxidase 50 (PER50) (P50) (PRXR2) | 2.37 |
| At1g10370 | glutathione S-transferase, putative (ERD9) | 2.37 |
| At4g22870 | leucoanthocyanidin dioxygenase, putative/anthocyanidin synthase, | 2.36 |
| | putative | |
| At2g03600 | expressed protein | 2.36 |
| At5g35510 | hypothetical protein | 2.36 |
| At2g39420 | esterase/lipase/thioesterase family protein | 2.35 |
| At1g19640 | S-adenosyl-L-methionine:jasmonic acid carboxyl methyltransferase | 2.35 |
| | (JMT) | |
| At4g16730 | terpene synthase/cyclase family protein | 2.35 |
| At1g44350 | IAA-amino acid hydrolase 6, putative (ILL6)/IAA-Ala hydrolase, putative | 2.34 |
| At1g71140 | MATE efflux family protein | 2.33 |
| At1g52890 | no apical meristem (NAM) family protein | 2.33 |
| At2g16660 | nodulin family protein | 2.31 |
| At4g12480 | protease inhibitor/seed storage/lipid transfer protein (LTP) family protein | 2.30 |
| At1g26920 | expressed protein | 2.29 |
| At1g60030 | xanthine/uracil permease family protein | 2.29 |
| At1g55020 | lipoxygenase (LOX1) | 2.28 |
| At5g04490 | phosphatidate cytidylyltransferase family protein | 2.28 |
| At5g35480 | expressed protein | 2.27 |
| At3g56080 | dehydration-responsive protein-related | 2.27 |
| At2g46640 | hypothetical protein | 2.26 |
| At2g40880 | cysteine protease inhibitor, putative/cystatin, putative (FL3-27) | 2.26 |
| At2g43535 | trypsin inhibitor, putative | 2.25 |
| At1g22550 | proton-dependent oligopeptide transport (POT) family protein | 2.22 |
| At2g36590 | proline transporter, putative | 2.22 |
| At2g24580 | sarcosine oxidase family protein | 2.22 |
| At4g16590 | glucosyltransferase-related | 2.22 |
| At2g15960 | expressed protein | 2.21 |
| At1g13080 | cytochrome P450 family protein | 2.20 |
| At5g45960 | GDSL-motif lipase/hydrolase family protein | 2.20 |
| At3g55110 | ABC transporter family protein | 2.20 |
| At3g57680 | peptidase S41 family protein | 2.19 |
| At5g15960 | stress-responsive protein (KIN1) | 2.19 |
| At4g08870 | arginase, putative /// arginase, putative | 2.19 |
| At5g65390 | arabinogalactan-protein (AGP7) | 2.17 |
| At4g11650 | osmotin-like protein (OSM34) | 2.17 |
| At2g22470 | arabinogalactan-protein (AGP2) | 2.17 |
| At1g56580 | expressed protein | 2.17 |
| At4g17500 | ethylene-responsive element-binding protein 1 (ERF1)/EREBP-2 | 2.17 |
| | protein | |
| At3g62950 | glutaredoxin family protein | 2.16 |
| At4g01080 | expressed protein | 2.16 |
| At1g75580 | auxin-responsive protein, putative | 2.16 |
| At1g76090 | S-adenosyl-methionine-sterol-C-methyltransferase | 2.15 |
| At1g69490 | no apical meristem (NAM) family protein | 2.15 |
| At4g15660 | glutaredoxin family protein | 2.14 |
| At3g29320 | glucan phosphorylase, putative | 2.13 |
| At5g54060 | glycosyltransferase family protein | 2.13 |
| At4g37800 | xyloglucan:xyloglucosyl transferase, putative/xyloglucan | 2.12 |
| | endotransglycosylase, putative/endo-xyloglucan transferase, putative | |
| At1g03020 | glutaredoxin family protein | 2.12 |
| At1g75450 | FAD-binding domain-containing protein/cytokinin oxidase family protein | 2.11 |
| At3g46660 | UDP-glucoronosyl/UDP-glucosyl transferase family protein | 2.11 |
| At1g70820 | phosphoglucomutase, putative/glucose phosphomutase, putative | 2.11 |
| At1g17970 | zinc finger (C3HC4-type RING finger) family protein | 2.10 |
| At4g16690 | esterase/lipase/thioesterase family protein | 2.10 |
| At5g06870 | polygalacturonase inhibiting protein 2 (PGIP2) | 2.10 |
| At4g27410 | no apical meristem (NAM) family protein (RD26) | 2.09 |
| At3g01420 | pathogen-responsive alpha-dioxygenase, putative | 2.09 |
| At1g05340 | expressed protein | 2.09 |
| At1g29395 | stress-responsive protein, putative | 2.09 |
| At3g11900 | amino acid transporter family protein | 2.09 |
| At4g21620 | glycine-rich protein | 2.08 |
| At4g18440 | adenylosuccinate lyase, putative/adenylosuccinase, putative | 2.08 |
| At1g20350 | mitochondrial import inner membrane translocase subunit Tim17, | 2.08 |
| | putative | |
| At1g74010 | strictosidine synthase family protein | 2.08 |
| At3g61060 | F-box family protein/lectin-related | 2.07 |
| At3g07350 | expressed protein | 2.07 |
| At5g13400 | proton-dependent oligopeptide transport (POT) family protein | 2.07 |
| At1g06570 | 4-hydroxyphenylpyruvate dioxygenase (HPD) | 2.05 |
| At1g76790 | O-methyltransferase family 2 protein | 2.05 |
| At1g71080 | expressed protein | 2.05 |
| At5g57550 | xyloglucan:xyloglucosyl transferase/xyloglucan endotransglycosylase/ | 2.04 |
| | endo-xyloglucan transferase (XTR3) | |
| At2g37180 | plasma membrane intrinsic protein 2B (PIP2B)/aquaporin PIP2.2 | 2.04 |
| | (PIP2.2) | |
| At4g24380 | expressed protein | 2.04 |
| At2g28190 | superoxide dismutase (Cu—Zn), chloroplast (SODCP)/copper/zinc | 2.03 |
| | superoxide dismutase (CSD2) | |
| At5g45840 | leucine-rich repeat transmembrane protein kinase, putative | 2.03 |
| At2g37760 | aldo/keto reductase family protein | 2.03 |
| At2g26690 | nitrate transporter (NTP2) | 2.02 |
| At2g36870 | xyloglucan:xyloglucosyl transferase, putative/xyloglucan | 2.02 |
| | endotransglycosylase, putative/endo-xyloglucan transferase, putative | |
| At5g48180 | kelch repeat-containing protein | 2.02 |
| At5g54160 | quercetin 3-O-methyltransferase 1/flavonol 3-O-methyltransferase 1/ | 2.02 |
| | caffeic acid/5-hydroxyferulic acid O-methyltransferase (OMT1) | |
| At1g60190 | armadillo/beta-catenin repeat family protein/U-box domain-containing | 2.02 |
| | protein | |
| At4g14130 | xyloglucan:xyloglucosyl transferase, putative/xyloglucan | 2.02 |
| | endotransglycosylase, putative/endo-xyloglucan transferase, putative | |
| | (XTR7) | |
| At3g14060 | expressed protein /// expressed protein | 2.01 |
| At3g23410 | alcohol oxidase-related | 2.01 |
| At1g54040 | kelch repeat-containing protein | 2.01 |
| At1g12520 | superoxide dismutase copper chaperone, putative | 2.01 |
| At3g05180 | GDSL-motif lipase/hydrolase family protein | 2.01 |
| At5g65870 | phytosulfokines 5 (PSK5) | 2.01 |
| At3g50770 | calmodulin-related protein, putative | 2.00 |
| At5g02580 | expressed protein | 2.00 |
| At5g24120 | RNA polymerase sigma subunit SigE (sigE)/sigma-like factor (SIG5) | 2.00 |
| At5g13220 | expressed protein | 2.00 |
| At1g56670 | GDSL-motif lipase/hydrolase family protein | 2.00 |
| At1g24150 | formin homology 2 domain-containing protein/FH2 domain-containing | −2.02 |
| | protein | |
| At3g05730 | expressed protein | −2.02 |
| At4g24190 | shepherd protein (SHD)/clavata formation protein, putative | −2.03 |
| At4g27300 | S-locus protein kinase, putative | −2.03 |
| At5g07030 | aspartyl protease family protein | −2.04 |
| At1g27030 | expressed protein | −2.04 |
| At5g04950 | nicotianamine synthase, putative | −2.04 |
| At5g11670 | malate oxidoreductase, putative | −2.05 |
| At2g31060 | elongation factor family protein | −2.05 |
| At2g33210 | chaperonin, putative | −2.06 |
| At5g54610 | ankyrin repeat family protein | −2.06 |
| At2g36970 | UDP-glucoronosyl/UDP-glucosyl transferase family protein | −2.06 |
| At3g57260 | glycosyl hydrolase family 17 protein | −2.07 |
| At4g16870 | — | −2.07 |
| At2g22500 | mitochondrial substrate carrier family protein | −2.07 |
| At1g35230 | arabinogalactan-protein (AGP5) | −2.07 |
| At2g41180 | sigA-binding protein-related | −2.07 |
| At2g16060 | non-symbiotic hemoglobin 1 (HB1) (GLB1) | −2.08 |
| At3g15400 | anther development protein, putative | −2.08 |
| At1g03210 | phenazine biosynthesis PhzC/PhzF family protein | −2.08 |
| At3g09940 | monodehydroascorbate reductase, putative | −2.08 |
| At2g24850 | aminotransferase, putative | −2.10 |
| At5g60950 | phytochelatin synthetase-related | −2.11 |
| At1g76960 | expressed protein | −2.11 |
| At1g48210 | serine/threonine protein kinase, putative | −2.11 |
| At2g02130 | plant defensin-fusion protein, putative (PDF2.3) | −2.12 |
| At4g23130 | receptor-like protein kinase 6 (RLK6) | −2.12 |
| At2g26020 | plant defensin-fusion protein, putative (PDF1.2b) | −2.12 |
| At1g56120 | leucine-rich repeat family protein/protein kinase family protein | −2.14 |
| At1g08050 | zinc finger (C3HC4-type RING finger) family protein | −2.15 |
| At4g18250 | receptor serine/threonine kinase, putative | −2.16 |
| At2g38940 | phosphate transporter (PT2) | −2.16 |
| At1g01340 | cyclic nucleotide-regulated ion channel (CNGC10) (ACBK1) | −2.17 |
| At3g05650 | disease resistance family protein | −2.17 |
| At5g08260 | serine carboxypeptidase S10 family protein | −2.17 |
| At1g08450 | calreticulin 3 (CRT3) | −2.18 |
| At1g57630 | disease resistance protein (TIR class), putative | −2.18 |
| At1g19960 | expressed protein | −2.18 |
| At4g14400 | ankyrin repeat family protein | −2.19 |
| At3g02040 | glycerophosphoryl diester phosphodiesterase family protein | −2.20 |
| At5g62210 | embryo-specific protein-related | −2.21 |
| At3g24900 | disease resistance family protein/LRR family protein | −2.21 |
| At2g14610 | pathogenesis-related protein 1 (PR-1) | −2.22 |
| At5g54720 | ankyrin repeat family protein | −2.23 |
| At3g50480 | broad-spectrum mildew resistance RPW8 family protein | −2.23 |
| At5g52810 | ornithine cyclodeaminase/mu-crystallin family protein | −2.23 |
| At2g34260 | transducin family protein/WD-40 repeat family protein | −2.24 |
| At2g22170 | lipid-associated family protein | −2.24 |
| At3g53980 | protease inhibitor/seed storage/lipid transfer protein (LTP) family protein | −2.25 |
| At5g25260 | expressed protein | −2.25 |
| At4g20780 | calcium-binding protein, putative | −2.25 |
| At5g44420 | plant defensin protein, putative (PDF1.2a) | −2.26 |
| At4g21830 | methionine sulfoxide reductase domain-containing protein/SelR | −2.26 |
| | domain-containing protein | |
| At2g20870 | cell wall protein precursor, putative | −2.26 |
| At4g23200 | protein kinase family protein | −2.27 |
| At3g26210 | cytochrome P450 71B23, putative (CYP71B23) | −2.27 |
| At5g39670 | calcium-binding EF hand family protein | −2.27 |
| At3g60420 | expressed protein | −2.27 |
| At2g40750 | WRKY family transcription factor | −2.28 |
| At5g02760 | protein phosphatase 2C family protein/PP2C family protein | −2.28 |
| At3g44750 | histone deacetylase, putative (HD2A) | −2.30 |
| At1g80270 | DNA-binding protein, putative | −2.30 |
| At1g10340 | ankyrin repeat family protein | −2.31 |
| At1g24020 | Bet v I allergen family protein | −2.31 |
| At5g20400 | oxidoreductase, 2OG-Fe(II) oxygenase family protein | −2.32 |
| At3g51330 | aspartyl protease family protein | −2.32 |
| At5g58940 | protein kinase family protein | −2.34 |
| At1g35710 | leucine-rich repeat transmembrane protein kinase, putative | −2.34 |
| At5g45380 | sodium:solute symporter family protein | −2.35 |
| At4g11850 | phospholipase D gamma 1/PLD gamma 1 (PLDGAMMA1) | −2.37 |
| At4g00700 | C2 domain-containing protein | −2.39 |
| At5g52310 | low-temperature-responsive protein 78 (LTI78)/desiccation-responsive | −2.39 |
| | protein 29A (RD29A) | |
| At5g22580 | expressed protein | −2.40 |
| At3g05660 | disease resistance family protein | −2.40 |
| At5g12940 | leucine-rich repeat family protein | −2.42 |
| At4g34135 | UDP-glucoronosyl/UDP-glucosyl transferase family protein | −2.42 |
| At1g52200 | expressed protein | −2.42 |
| At4g23220 | protein kinase family protein | −2.46 |
| At2g04450 | MutT/nudix family protein | −2.47 |
| At1g67990 | caffeoyl-CoA 3-O-methyltransferase, putative | −2.50 |
| At4g23150 | protein kinase family protein | −2.50 |
| At1g21250 | wall-associated kinase 1 (WAK1) | −2.52 |
| At5g28540 | luminal binding protein 1 (BiP-1) (BP1) | −2.53 |
| At3g09020 | alpha 1,4-glycosyltransferase family protein/glycosyltransferase sugar- | −2.54 |
| | binding DXD motif-containing protein | |
| At2g02930 | glutathione S-transferase, putative | −2.55 |
| At5g61160 | transferase family protein | −2.55 |
| At2g02810 | UDP-galactose/UDP-glucose transporter | −2.56 |
| At3g48640 | expressed protein | −2.58 |
| At4g21380 | S-locus protein kinase, putative (ARK3) | −2.58 |
| At4g14365 | zinc finger (C3HC4-type RING finger) family protein/ankyrin repeat | −2.61 |
| | family protein | |
| At3g01290 | band 7 family protein | −2.63 |
| At1g30900 | vacuolar sorting receptor, putative | −2.64 |
| At2g26440 | pectinesterase family protein | −2.64 |
| At4g02380 | late embryogenesis abundant 3 family protein/LEA3 family protein | −2.67 |
| At1g13470 | expressed protein | −2.71 |
| At3g23110 | disease resistance family protein /// disease resistance family protein | −2.73 |
| At5g64510 | expressed protein | −2.74 |
| At5g59670 | leucine-rich repeat protein kinase, putative | −2.75 |
| At1g75040 | pathogenesis-related protein 5 (PR-5) | −2.76 |
| At5g42900 | expressed protein | −2.77 |
| At1g66850 | protease inhibitor/seed storage/lipid transfer protein (LTP) family protein | −2.78 |
| At4g01700 | chitinase, putative | −2.79 |
| At1g77510 | protein disulfide isomerase, putative | −2.82 |
| At5g07550 | glycine-rich protein (GRP19) | −2.84 |
| At1g31690 | copper amine oxidase, putative | −2.87 |
| At3g25010 | disease resistance family protein | −2.90 |
| At1g75940 | glycosyl hydrolase family 1 protein/anther-specific protein ATA27 | −2.93 |
| At1g04980 | thioredoxin family protein | −2.94 |
| At4g23140 | receptor-like protein kinase 5 (RLK5) | −2.95 |
| At2g47130 | short-chain dehydrogenase/reductase (SDR) family protein | −2.97 |
| At5g20630 | germin-like protein (GER3) | −3.01 |
| At4g11890 | protein kinase family protein | −3.03 |
| At1g68875 | expressed protein | −3.04 |
| At4g10500 | oxidoreductase, 2OG-Fe(II) oxygenase family protein | −3.17 |
| At5g52760 | heavy-metal-associated domain-containing protein | −3.20 |
| At3g48080 | lipase class 3 family protein/disease resistance protein-related | −3.31 |
| At5g52740 | heavy-metal-associated domain-containing protein | −3.31 |
| At5g59680 | leucine-rich repeat protein kinase, putative | −3.34 |
| At4g17030 | expansin-related | −3.35 |
| At3g16530 | legume lectin family protein | −3.57 |
| At4g03450 | ankyrin repeat family protein | −3.60 |
| At2g26560 | patatin, putative | −3.60 |
| At3g23550 | MATE efflux family protein | −3.61 |
| At1g14870 | expressed protein | −3.73 |
| At1g67810 | Fe—S metabolism associated domain-containing protein | −3.77 |
| At3g11010 | disease resistance family protein/LRR family protein | −3.82 |
| At3g16670 | expressed protein | −3.86 |
| At5g55450 | protease inhibitor/seed storage/lipid transfer protein (LTP) family protein | −3.90 |
| At5g20790 | expressed protein | −3.93 |
| At2g14560 | expressed protein | −3.96 |
| At1g09080 | luminal binding protein 3 (BiP-3) (BP3) | −4.12 |
| At4g25110 | latex-abundant family protein (AMC2)/caspase family protein | −4.21 |
| At4g08300 | nodulin MtN21 family protein | −4.23 |
| At5g18470 | curculin-like (mannose-binding) lectin family protein | −4.23 |
| At3g51590 | lipid transfer protein, putative | −5.14 |
| At1g29720 | protein kinase family protein | −5.41 |
| At3g57240 | beta-1,3-glucanase (BG3) | −5.50 |
| At5g23020 | 2-isopropylmalate synthase 2 (IMS2) | −8.88 |

TABLE 2
Genes significantly up or down regulated 2-fold in Arabidopsis overexpressing
(OE) wheat GLK1 (TaGLK1). Data analysed using FlexArray for normalization and T-Test

| | | TaGLK1 |
|---|---|---|
| | | OE Fold |
| Transcript ID | Gene Title | change |
| At3g08770 | lipid transfer protein 6 (LTP6) | 74.83 |
| At2g33850 | expressed protein | 63.70 |
| At1g72260 | thionin (THI2.1) | 47.99 |
| At2g42840 | protodermal factor 1 (PDF1) | 47.96 |
| At3g09260 | glycosyl hydrolase family 1 protein | 26.10 |
| At4g37410 | cytochrome P450, putative | 25.94 |
| At1g62500 | protease inhibitor/seed storage/lipid transfer protein (LTP) family protein | 25.44 |
| At5g45670 | GDSL-motif lipase/hydrolase family protein | 24.42 |
| At4g22490 | protease inhibitor/seed storage/lipid transfer protein (LTP) family protein | 21.94 |
| At4g29030 | glycine-rich protein | 19.68 |
| At1g77960 | Phox-bem1p domain protein | 18.56 |
| At2g28790 | osmotin-like protein, putative | 16.93 |
| At5g36910 | thionin (THI2.2) | 16.22 |
| At2g20870 | cell wall protein precursor, putative | 15.83 |
| At1g54010 | myrosinase-associated protein, putative | 15.49 |
| At1g28290 | pollen Ole e 1 allergen and extensin family protein | 15.44 |
| At1g54020 | myrosinase-associated protein, putative | 14.89 |
| At5g23940 | transferase family protein | 14.76 |
| At5g33370 | GDSL-motif lipase/hydrolase family protein | 14.45 |
| At3g15950 | DNA topoisomerase-related | 12.88 |
| At5g08000 | glycosyl hydrolase family protein 17 | 12.68 |
| At5g42800 | dihydroflavonol 4-reductase (dihydrokaempferol 4-reductase) (DFR) | 12.64 |
| At3g16430 | jacalin lectin family protein | 12.56 |
| At4g29700 | type I phosphodiesterase/nucleotide pyrophosphatase family protein | 11.56 |
| At5g24420 | glucosamine/galactosamine-6-phosphate isomerase-related | 11.34 |
| At2g39310 | jacalin lectin family protein | 10.72 |
| At4g19430 | expressed protein | 10.34 |
| At1g24020 | Bet v I allergen family protein | 10.18 |
| At4g22870 | leucoanthocyanidin dioxygenase, putative/anthocyanidin synthase, | 10.13 |
| | putative | |
| At3g02550 | LOB domain protein 41/lateral organ boundaries domain protein 41 | 10.00 |
| | (LBD41) | |
| At4g17470 | palmitoyl protein thioesterase family protein | 9.67 |
| At4g23680 | major latex protein-related/MLP-related | 8.76 |
| At5g20740 | invertase/pectin methylesterase inhibitor family protein | 8.74 |
| At1g18250 | thaumatin, putative | 8.70 |
| At2g14900 | gibberellin-regulated family protein | 8.65 |
| At4g11310 | cysteine proteinase, putative | 8.65 |
| At1g76790 | O-methyltransferase family 2 protein | 8.45 |
| At5g07030 | aspartyl protease family protein | 8.32 |
| At1g08560 | syntaxin-related protein KNOLLE (KN)/syntaxin 111 (SYP111) | 8.12 |
| At5g23820 | MD-2-related lipid recognition domain-containing protein/ML domain- | 8.06 |
| | containing protein | |
| At5g28640 | SSXT protein-related/glycine-rich protein | 7.97 |
| At4g23800 | high mobility group (HMG1/2) family protein | 7.95 |
| At5g47500 | pectinesterase family protein | 7.92 |
| At4g33260 | WD-40 repeat family protein | 7.86 |
| At2g21140 | hydroxyproline-rich glycoprotein family protein | 7.85 |
| At3g02640 | expressed protein | 7.83 |
| At5g16250 | expressed protein | 7.75 |
| At1g02730 | cellulose synthase family protein | 7.68 |
| At1g31710 | copper amine oxidase, putative | 7.55 |
| At1g10640 | polygalacturonase, putative/pectinase, putative | 7.55 |
| At3g55970 | oxidoreductase, 2OG-Fe(II) oxygenase family protein | 7.33 |
| At5g55180 | glycosyl hydrolase family 17 protein | 7.27 |
| At5g17220 | glutathione S-transferase, putative | 7.26 |
| At2g38530 | nonspecific lipid transfer protein 2 (LTP2) | 7.21 |
| At4g15910 | drought-responsive protein/drought-induced protein (Di21) | 7.11 |
| At4g02290 | glycosyl hydrolase family 9 protein | 7.07 |
| At3g22880 | meiotic recombination protein, putative | 6.86 |
| At5g54060 | glycosyltransferase family protein | 6.85 |
| At2g43520 | trypsin inhibitor, putative | 6.81 |
| At4g31840 | plastocyanin-like domain-containing protein | 6.81 |
| At3g17680 | expressed protein | 6.68 |
| At3g48350 | cysteine proteinase, putative | 6.66 |
| At4g12490 | protease inhibitor/seed storage/lipid transfer protein (LTP) family protein | 6.61 |
| At4g15830 | expressed protein | 6.58 |
| At3g16450 | jacalin lectin family protein | 6.54 |
| At1g18370 | kinesin motor family protein (NACK1) | 6.50 |
| At2g02850 | plastocyanin-like domain-containing protein/plantacyanin, putative | 6.38 |
| At1g03495 | pseudogene, transferase family | 6.17 |
| At1g06100 | fatty acid desaturase family protein | 6.13 |
| At4g12500 | protease inhibitor/seed storage/lipid transfer protein (LTP) family protein | 6.13 |
| At4g34160 | cyclin delta-3 (CYCD3) | 6.11 |
| At2g38870 | protease inhibitor, putative | 6.07 |
| At2g23000 | serine carboxypeptidase S10 family protein | 6.04 |
| At3g05600 | epoxide hydrolase, putative | 6.01 |
| At1g19610 | plant defensin-fusion protein, putative (PDF1.4) | 5.92 |
| At3g16390 | jacalin lectin family protein | 5.90 |
| At5g51750 | subtilase family protein | 5.87 |
| At1g13710 | cytochrome P450 family protein | 5.72 |
| At1g70710 | endo-1,4-beta-glucanase (EGASE)/cellulase | 5.71 |
| At1g43800 | acyl-(acyl-carrier-protein) desaturase, putative/stearoyl-ACP | 5.67 |
| | desaturase, putative | |
| At3g15720 | glycoside hydrolase family 28 protein/polygalacturonase (pectinase) | 5.67 |
| At3g46640 | myb family transcription factor | 5.63 |
| At4g29020 | glycine-rich protein | 5.56 |
| At3g21770 | peroxidase 30 (PER30) (P30) (PRXR9) | 5.51 |
| At5g04530 | beta-ketoacyl-CoA synthase family protein | 5.46 |
| At3g02120 | hydroxyproline-rich glycoprotein family protein | 5.45 |
| At1g80080 | leucine-rich repeat family protein | 5.44 |
| At4g32830 | protein kinase, putative | 5.41 |
| At2g25880 | serine/threonine protein kinase, putative | 5.40 |
| At5g17700 | MATE efflux family protein | 5.40 |
| At4g02850 | phenazine biosynthesis PhzC/PhzF family protein | 5.39 |
| At1g52040 | myrosinase-binding protein, putative (F-ATMBP) | 5.37 |
| At4g02800 | expressed protein | 5.34 |
| At1g14900 | high-mobility-group protein/HMG-I/Y protein | 5.33 |
| At5g43020 | leucine-rich repeat transmembrane protein kinase, putative | 5.31 |
| At5g22430 | expressed protein | 5.26 |
| At1g75240 | zinc finger homeobox family protein/ZF-HD homeobox family protein | 5.26 |
| At2g34490 | cytochrome P450 family protein | 5.25 |
| At5g37300 | expressed protein | 5.24 |
| At3g47340 | glutamine-dependent asparagine synthetase 1 (ASN1) | 5.24 |
| At4g05520 | calcium-binding EF hand family protein | 5.21 |
| At2g28210 | carbonic anhydrase family protein | 5.21 |
| At3g50070 | cyclin family protein | 5.19 |
| At1g44110 | cyclin, putative | 5.12 |
| At3g16470 | jacalin lectin family protein | 5.10 |
| At2g21640 | expressed protein | 5.10 |
| At5g01600 | ferritin 1 (FER1) | 5.06 |
| At2g43800 | formin homology 2 domain-containing protein/FH2 domain-containing | 5.04 |
| | protein | |
| At3g18000 | phosphoethanolamine N-methyltransferase 1/PEAMT 1 (NMT1) | 5.03 |
| At5g13840 | WD-40 repeat family protein | 5.01 |
| At1g14440 | zinc finger homeobox family protein/ZF-HD homeobox family protein | 5.00 |
| At3g28290 | integrin-related protein 14a | 4.98 |
| At3g04290 | GDSL-motif lipase/hydrolase family protein | 4.89 |
| At4g02360 | expressed protein | 4.86 |
| At5g25090 | plastocyanin-like domain-containing protein | 4.82 |
| At3g25980 | mitotic spindle checkpoint protein, putative (MAD2) | 4.80 |
| At2g38750 | annexin 4 (ANN4) | 4.78 |
| At2g42610 | expressed protein | 4.78 |
| At3g28500 | 60S acidic ribosomal protein P2 (RPP2C) | 4.77 |
| At3g28740 | cytochrome P450 family protein | 4.76 |
| At1g01600 | cytochrome P450, putative | 4.74 |
| At3g53190 | pectate lyase family protein | 4.74 |
| At4g14090 | UDP-glucoronosyl/UDP-glucosyl transferase family protein | 4.73 |
| At3g23890 | DNA topoisomerase, ATP-hydrolyzing/DNA topoisomerase II/DNA | 4.71 |
| | gyrase (TOP2) | |
| At1g22770 | gigantea protein (GI) | 4.70 |
| At1g64200 | vacuolar ATP synthase subunit E, putative | 4.68 |
| At5g54510 | auxin-responsive GH3 protein, putative (DFL-1) | 4.61 |
| At5g36710 | expressed protein | 4.60 |
| At1g04110 | subtilase family protein | 4.56 |
| At1g54050 | 17.4 kDa class III heat shock protein (HSP17.4-CIII) | 4.56 |
| At4g37750 | ovule development protein aintegumenta (ANT) | 4.56 |
| At3g55110 | ABC transporter family protein | 4.53 |
| At1g24070 | glycosyl transferase family 2 protein | 4.52 |
| At5g38120 | 4-coumarate--CoA ligase family protein/4-coumaroyl-CoA synthase | 4.50 |
| | family protein | |
| At4g12480 | protease inhibitor/seed storage/lipid transfer protein (LTP) family protein | 4.45 |
| At5g01870 | lipid transfer protein, putative | 4.43 |
| At2g25060 | plastocyanin-like domain-containing protein | 4.43 |
| At4g39510 | cytochrome P450 family protein | 4.42 |
| At4g14770 | tesmin/TSO1-like CXC domain-containing protein | 4.42 |
| At4g12470 | protease inhibitor/seed storage/lipid transfer protein (LTP) family protein | 4.38 |
| At5g48485 | protease inhibitor/seed storage/lipid transfer protein (LTP) family protein | 4.35 |
| At1g07050 | CONSTANS-like protein-related | 4.32 |
| At1g73620 | thaumatin-like protein, putative/pathogenesis-related protein, putative | 4.30 |
| At5g57785 | expressed protein | 4.30 |
| At1g79840 | homeobox-leucine zipper protein 10 (HB-10)/HD-ZIP transcription | 4.29 |
| | factor 10/homeobox protein (GLABRA2) | |
| At2g19590 | 1-aminocyclopropane-1-carboxylate oxidase, putative/ACC oxidase, | 4.28 |
| | putative | |
| At1g52410 | caldesmon-related | 4.26 |
| At5g44560 | SNF7 family protein | 4.26 |
| At2g21650 | myb family transcription factor | 4.24 |
| At5g13220 | expressed protein | 4.23 |
| At2g02100 | plant defensin-fusion protein, putative (PDF2.2) | 4.23 |
| At1g76540 | cell division control protein, putative | 4.18 |
| At5g64080 | protease inhibitor/seed storage/lipid transfer protein (LTP) family protein | 4.17 |
| At1g62360 | homeobox protein SHOOT MERISTEMLESS (STM) | 4.17 |
| At1g12845 | expressed protein | 4.17 |
| At3g51280 | male sterility MS5, putative | 4.17 |
| At3g14190 | expressed protein | 4.16 |
| At1g48330 | expressed protein | 4.15 |
| At5g19110 | extracellular dermal glycoprotein-related/EDGP-related | 4.08 |
| At2g39330 | jacalin lectin family protein | 4.08 |
| At1g50490 | ubiquitin-conjugating enzyme 20 (UBC20) | 4.04 |
| At4g27860 | integral membrane family protein | 4.04 |
| At1g25450 | very-long-chain fatty acid condensing enzyme, putative | 4.03 |
| At4g30140 | GDSL-motif lipase/hydrolase family protein | 3.99 |
| At2g23170 | auxin-responsive GH3 family protein | 3.99 |
| At1g67750 | pectate lyase family protein | 3.96 |
| At1g72970 | glucose-methanol-choline (GMC) oxidoreductase family protein | 3.95 |
| At1g70830 | Bet v I allergen family protein | 3.95 |
| At3g13175 | expressed protein | 3.95 |
| At4g34250 | fatty acid elongase, putative | 3.93 |
| At1g51460 | ABC transporter family protein | 3.93 |
| At2g38760 | annexin 3 (ANN3) | 3.92 |
| At5g15230 | gibberellin-regulated protein 4 (GASA4) | 3.91 |
| At3g16460 | jacalin lectin family protein | 3.91 |
| At2g43620 | chitinase, putative | 3.90 |
| At1g56650 | myb family transcription factor (MYB75) | 3.90 |
| At3g11600 | expressed protein | 3.89 |
| At2g40300 | ferritin, putative | 3.89 |
| At2g22840 | expressed protein | 3.89 |
| At3g23730 | xyloglucan:xyloglucosyl transferase, putative | 3.88 |
| At4g03210 | xyloglucan:xyloglucosyl transferase, putative | 3.87 |
| At1g66370 | myb family transcription factor (MYB113) | 3.86 |
| At1g62480 | vacuolar calcium-binding protein-related | 3.85 |
| At3g29590 | transferase family protein | 3.85 |
| At4g11650 | osmotin-like protein (OSM34) | 3.85 |
| At1g20030 | pathogenesis-related thaumatin family protein | 3.84 |
| At1g06350 | fatty acid desaturase family protein | 3.83 |
| At4g11190 | disease resistance-responsive family protein/dirigent family protein | 3.80 |
| At3g52110 | expressed protein | 3.80 |
| At3g01140 | myb family transcription factor (MYB106) | 3.79 |
| At5g58390 | peroxidase, putative | 3.79 |
| At3g06030 | NPK1-related protein kinase, putative (ANP3) | 3.79 |
| At1g55260 | protease inhibitor/seed storage/lipid transfer protein (LTP) family protein | 3.76 |
| At1g10522 | expressed protein | 3.75 |
| At3g17990 | phosphoethanolamine N-methyltransferase 1/PEAMT 1 (NMT1) | 3.75 |
| At5g60890 | receptor-like protein kinase (ATR1) (MYB34) | 3.75 |
| At4g00480 | myc-related transcription factor (MYC1) | 3.72 |
| At1g50240 | armadillo/beta-catenin repeat family protein | 3.72 |
| At2g39030 | GCN5-related N-acetyltransferase (GNAT) family protein | 3.70 |
| At1g17860 | trypsin and protease inhibitor family protein/Kunitz family | 3.70 |
| At4g38660 | thaumatin, putative | 3.69 |
| At3g23830 | glycine-rich RNA-binding protein, putative | 3.69 |
| At3g21300 | RNA methyltransferase family protein | 3.66 |
| At2g37640 | expansin, putative (EXP3) | 3.64 |
| At5g17160 | expressed protein | 3.64 |
| At2g42570 | expressed protein | 3.63 |
| At4g03100 | rac GTPase activating protein, putative | 3.62 |
| At5g16190 | glycosyl transferase family 2 protein | 3.60 |
| At1g53885 | senescence-associated protein-related | 3.60 |
| At4g37740 | expressed protein | 3.59 |
| At1g78490 | cytochrome P450 family protein | 3.58 |
| At1g17380 | expressed protein | 3.58 |
| At1g73260 | trypsin and protease inhibitor family protein/Kunitz family protein | 3.57 |
| At1g74430 | myb family transcription factor (MYB95) | 3.54 |
| At5g67270 | microtubule-associated EB1 family protein | 3.53 |
| At4g31290 | ChaC-like family protein | 3.52 |
| At5g23310 | superoxide dismutase (Fe)/iron superoxide dismutase 3 (FSD3) | 3.51 |
| At1g75780 | tubulin beta-1 chain (TUB1) | 3.50 |
| At1g33170 | dehydration-responsive family protein | 3.49 |
| At3g56070 | peptidyl-prolyl cis-trans isomerase, putative | 3.45 |
| At5g60100 | pseudo-response regulator 3 (APRR3) | 3.44 |
| At1g03780 | targeting protein-related | 3.43 |
| At3g44990 | xyloglucan:xyloglucosyl transferase, putative/xyloglucan | 3.41 |
| | endotransglycosylase, putative/endo-xyloglucan transferase, putative | |
| At5g45700 | NLI interacting factor (NIF) family protein | 3.41 |
| At2g34810 | FAD-binding domain-containing protein | 3.41 |
| At1g69200 | pfkB-type carbohydrate kinase family protein | 3.39 |
| At1g50010 | tubulin alpha-2/alpha-4 chain (TUA2) | 3.39 |
| At5g48360 | formin homology 2 domain-containing protein/FH2 domain protein | 3.38 |
| At5g37010 | expressed protein | 3.38 |
| At3g48500 | expressed protein | 3.37 |
| At3g12500 | basic endochitinase | 3.37 |
| At3g54820 | aquaporin, putative | 3.37 |
| At5g39790 | 5′-AMP-activated protein kinase beta-1 subunit-related | 3.34 |
| At3g27690 | chlorophyll A-B binding protein (LHCB2:4) | 3.33 |
| At3g26330 | cytochrome P450 family protein | 3.32 |
| At5g65020 | annexin 2 (ANN2) | 3.32 |
| At1g02690 | importin alpha-2 subunit, putative | 3.32 |
| At5g22500 | acyl CoA reductase, putative/male-sterility protein, putative | 3.32 |
| At4g16590 | glucosyltransferase-related | 3.28 |
| At2g36570 | leucine-rich repeat transmembrane protein kinase, putative | 3.28 |
| At3g44750 | histone deacetylase, putative (HD2A) | 3.28 |
| At2g43510 | trypsin inhibitor, putative | 3.27 |
| At1g10780 | F-box family protein | 3.27 |
| At3g18960 | transcriptional factor B3 family protein | 3.27 |
| At1g34245 | expressed protein | 3.25 |
| At2g27510 | ferredoxin, putative | 3.25 |
| At3g12870 | expressed protein | 3.25 |
| At3g10570 | cytochrome P450, putative | 3.24 |
| At1g56580 | expressed protein | 3.24 |
| At5g52120 | F-box family protein/SKP1 interacting partner 3-related | 3.23 |
| At2g23560 | hydrolase, alpha/beta fold family protein | 3.23 |
| At5g62080 | protease inhibitor/seed storage/lipid transfer protein (LTP) family protein | 3.21 |
| At1g14120 | 2-oxoglutarate-dependent dioxygenase, putative | 3.20 |
| At3g51590 | lipid transfer protein, putative | 3.20 |
| At4g36180 | leucine-rich repeat family protein | 3.20 |
| At5g62550 | expressed protein | 3.19 |
| At4g29610 | cytidine deaminase, putative/cytidine aminohydrolase, putative | 3.19 |
| At2g32280 | expressed protein | 3.19 |
| At1g17560 | ribosomal protein L14 family protein | 3.16 |
| At2g21660 | glycine-rich RNA-binding protein (GRP7) | 3.15 |
| At5g65280 | lanthionine synthetase C-like family protein | 3.14 |
| At1g72670 | calmodulin-binding family protein | 3.14 |
| At1g14250 | nucleoside phosphatase family protein/GDA1/CD39 family protein | 3.14 |
| At2g06925 | phospholipase A2 family protein | 3.13 |
| At1g46264 | heat shock transcription factor family protein | 3.11 |
| At1g53035 | expressed protein | 3.11 |
| At5g25475 | expressed protein | 3.11 |
| At4g19400 | expressed protein | 3.11 |
| At1g66760 | MATE efflux family protein | 3.10 |
| At3g22760 | CXC domain containing TSO1-like protein 1 (SOL1) | 3.10 |
| At5g51720 | expressed protein | 3.10 |
| At3g11480 | S-adenosyl-L-methionine:carboxyl methyltransferase family protein | 3.10 |
| At4g27570 | glycosyltransferase family protein | 3.09 |
| At4g37490 | G2/mitotic-specific cyclin (CYC1) | 3.09 |
| At3g22790 | kinase interacting family protein | 3.09 |
| At3g56330 | N2,N2-dimethylguanosine tRNA methyltransferase family protein | 3.09 |
| At2g34640 | expressed protein | 3.08 |
| At1g31335 | expressed protein | 3.08 |
| At4g13410 | glycosyl transferase family 2 protein | 3.08 |
| At5g02810 | pseudo-response regulator 7 (APRR7) | 3.07 |
| At2g27970 | cyclin-dependent kinase, putative/CDK, putative | 3.07 |
| At3g23670 | phragmoplast-associated kinesin-related protein, putative | 3.07 |
| At2g26330 | leucine-rich repeat protein kinase, putative (ERECTA) | 3.06 |
| At5g55520 | expressed protein | 3.06 |
| At5g57590 | aminotransferase class III family protein | 3.05 |
| At3g44590 | 60S acidic ribosomal protein P2 (RPP2D) | 3.05 |
| At1g14890 | invertase/pectin methylesterase inhibitor family protein | 3.04 |
| At3g20260 | expressed protein | 3.03 |
| At2g45490 | protein kinase, putative | 3.03 |
| At3g05180 | GDSL-motif lipase/hydrolase family protein | 3.03 |
| At1g60270 | pseudogene, glycosyl hydrolase family 1 | 3.02 |
| At4g25630 | fibrillarin 2 (FIB2) | 3.02 |
| At1g27030 | expressed protein | 3.02 |
| At5g55120 | expressed protein | 3.01 |
| At2g29550 | tubulin beta-7 chain (TUB7) | 3.01 |
| At1g57820 | zinc finger (C3HC4-type RING finger) family protein | 3.01 |
| At4g36010 | pathogenesis-related thaumatin family protein | 3.01 |
| At5g15160 | bHLH family protein | 3.01 |
| At2g01610 | invertase/pectin methylesterase inhibitor family protein | 3.00 |
| At5g45950 | GDSL-motif lipase/hydrolase family protein | 2.99 |
| At5g44050 | MATE efflux family protein | 2.99 |
| At1g23000 | heavy-metal-associated domain-containing protein | 2.99 |
| At5g56580 | mitogen-activated protein kinase kinase (MAPKK), putative (MKK6) | 2.99 |
| At3g05020 | acyl carrier protein 1, chloroplast (ACP-1) | 2.99 |
| At1g02205 | CER1 protein | 2.98 |
| At5g48310 | expressed protein | 2.98 |
| At3g19590 | WD-40 repeat family protein/mitotic checkpoint protein, putative | 2.95 |
| At5g55730 | fasciclin-like arabinogalactan-protein (FLA1) | 2.95 |
| At1g13650 | expressed protein | 2.94 |
| At1g56300 | DNAJ heat shock N-terminal domain-containing protein | 2.94 |
| At1g02800 | endo-1,4-beta-glucanase/cellulase (CEL2) | 2.94 |
| At1g45130 | beta-galactosidase, putative/lactase, putative | 2.94 |
| At5g46690 | basic helix-loop-helix (bHLH) family protein | 2.93 |
| At2g47180 | galactinol synthase, putative | 2.93 |
| At4g10270 | wound-responsive family protein | 2.93 |
| At2g39900 | LIM domain-containing protein | 2.93 |
| At4g26760 | microtubule associated protein (MAP65/ASE1) family protein | 2.92 |
| At1g78430 | tropomyosin-related | 2.92 |
| At1g62540 | flavin-containing monooxygenase family protein/FMO family protein | 2.92 |
| At4g29360 | glycosyl hydrolase family 17 protein | 2.92 |
| At5g67260 | cyclin family protein | 2.91 |
| At1g44446 | chlorophyll a oxygenase (CAO)/chlorophyll b synthase | 2.91 |
| At5g22740 | glycosyl transferase family 2 protein | 2.90 |
| At2g36200 | kinesin motor protein-related | 2.89 |
| At3g17820 | glutamine synthetase (GS1) | 2.89 |
| At1g72250 | kinesin motor protein-related | 2.89 |
| At2g26760 | cyclin, putative | 2.89 |
| At5g11410 | protein kinase family protein | 2.89 |
| At5g10160 | beta-hydroxyacyl-ACP dehydratase, putative | 2.89 |
| At5g44620 | cytochrome P450 family protein | 2.88 |
| At2g19670 | protein arginine N-methyltransferase, putative | 2.88 |
| At3g19820 | cell elongation protein/DWARF1/DIMINUTO (DIM) | 2.88 |
| At1g73330 | protease inhibitor, putative (DR4) | 2.88 |
| At5g51600 | microtubule associated protein (MAP65/ASE1) family protein | 2.87 |
| At5g18790 | ribosomal protein L33 family protein | 2.87 |
| At5g09980 | expressed protein | 2.86 |
| At4g39950 | cytochrome P450 79B2, putative (CYP79B2) | 2.86 |
| At4g28250 | beta-expansin, putative (EXPB3) | 2.85 |
| At3g55660 | expressed protein | 2.85 |
| At3g04630 | expressed protein | 2.85 |
| At2g17740 | DC1 domain-containing protein | 2.85 |
| At4g31805 | WRKY family transcription factor | 2.84 |
| At4g28160 | hydroxyproline-rich glycoprotein family protein | 2.84 |
| At2g19970 | pathogenesis-related protein, putative | 2.84 |
| At4g32980 | homeobox protein (ATH1) | 2.84 |
| At2g23150 | NRAMP metal ion transporter 3 (NRAMP3) | 2.83 |
| At3g28220 | meprin and TRAF homology domain-containing protein/MATH domain- | 2.82 |
| | containing protein | |
| At2g41650 | expressed protein | 2.82 |
| At1g44350 | IAA-amino acid hydrolase 6, putative (ILL6) | 2.81 |
| At2g47630 | esterase/lipase/thioesterase family protein | 2.81 |
| At3g21950 | S-adenosyl-L-methionine:carboxyl methyltransferase family protein | 2.81 |
| At4g15440 | hydroperoxide lyase (HPL1) | 2.80 |
| At3g61250 | myb family transcription factor (MYB17) | 2.80 |
| At1g52000 | jacalin lectin family protein | 2.80 |
| At4g24175 | expressed protein | 2.80 |
| At3g13980 | expressed protein | 2.79 |
| At5g23530 | expressed protein | 2.79 |
| At5g24160 | squalene monooxygenase 1,2/squalene epoxidase 1,2 (SQP1,2) | 2.78 |
| At3g07320 | glycosyl hydrolase family 17 protein | 2.77 |
| At3g51720 | expressed protein | 2.77 |
| At1g76310 | cyclin, putative | 2.77 |
| At4g24610 | expressed protein | 2.76 |
| At3g05980 | expressed protein | 2.76 |
| At5g48480 | expressed protein | 2.75 |
| At3g12700 | aspartyl protease family protein | 2.75 |
| At2g44040 | dihydrodipicolinate reductase family protein /// dihydrodipicolinate | 2.74 |
| | reductase family protein | |
| At2g43535 | trypsin inhibitor, putative | 2.73 |
| At1g06180 | myb family transcription factor | 2.73 |
| At4g27260 | auxin-responsive GH3 family protein | 2.72 |
| At1g18650 | glycosyl hydrolase family protein 17 | 2.72 |
| At1g72040 | deoxynucleoside kinase family | 2.72 |
| At4g33400 | dem protein-related/defective embryo and meristems protein-related | 2.71 |
| At5g02550 | expressed protein | 2.71 |
| At4g11290 | peroxidase, putative | 2.71 |
| At3g15680 | zinc finger (Ran-binding) family protein | 2.71 |
| At2g02990 | ribonuclease 1 (RNS1) | 2.71 |
| At1g52400 | glycosyl hydrolase family 1 protein/beta-glucosidase, putative (BG1) | 2.71 |
| At2g16270 | expressed protein | 2.71 |
| At5g65640 | basic helix-loop-helix (bHLH) family protein | 2.71 |
| At4g35060 | heavy-metal-associated domain-containing protein/copper chaperone | 2.71 |
| | (CCH)-related | |
| At1g06040 | zinc finger (B-box type) family protein/salt-tolerance protein (STO) | 2.70 |
| At1g19850 | transcription factor MONOPTEROS (MP)/auxin-responsive protein | 2.70 |
| | (IAA24)/auxin response factor 5 (ARF5) | |
| At1g65900 | expressed protein | 2.70 |
| At3g05130 | expressed protein | 2.69 |
| At1g17420 | lipoxygenase, putative | 2.69 |
| At5g15960 | stress-responsive protein (KIN1) | 2.69 |
| At4g03070 | 2-oxoglutarate-dependent dioxygenase (AOP1.2) | 2.69 |
| At1g59540 | kinesin motor protein-related | 2.69 |
| At1g67830 | GDSL-motif lipase/hydrolase family protein | 2.69 |
| At3g49160 | pyruvate kinase family protein | 2.68 |
| At5g24470 | pseudo-response regulator 5 (APRR5) | 2.68 |
| At5g39520 | expressed protein | 2.68 |
| At1g44760 | universal stress protein (USP) family protein | 2.67 |
| At5g60880 | expressed protein | 2.67 |
| At3g54090 | pfkB-type carbohydrate kinase family protein | 2.67 |
| At3g16690 | nodulin MtN3 family protein | 2.66 |
| At3g11210 | GDSL-motif lipase/hydrolase family protein | 2.66 |
| At2g42800 | leucine-rich repeat family protein | 2.66 |
| At3g10310 | kinesin motor protein-related | 2.66 |
| At5g65410 | zinc finger homeobox family protein/ZF-HD homeobox family protein | 2.66 |
| At5g06980 | expressed protein | 2.66 |
| At2g38620 | cell divsion control protein, putative | 2.65 |
| At1g80270 | DNA-binding protein, putative | 2.65 |
| At3g57600 | AP2 domain-containing transcription factor, putative | 2.65 |
| At2g27840 | histone deacetylase-related/HD-related | 2.65 |
| At4g35850 | pentatricopeptide (PPR) repeat-containing protein | 2.65 |
| At1g71050 | heavy-metal-associated domain-containing protein/copper chaperone | 2.65 |
| | (CCH)-related | |
| At1g65620 | LOB domain protein 6/lateral organ boundaries domain protein 6 | 2.65 |
| | (LBD6)/asymmetric leaves2 (AS2) | |
| At5g50335 | expressed protein | 2.64 |
| At5g15510 | expressed protein | 2.64 |
| At1g31550 | GDSL-motif lipase, putative | 2.64 |
| At1g78600 | zinc finger (B-box type) family protein | 2.63 |
| At5g61880 | signaling molecule-related | 2.63 |
| At5g64170 | dentin sialophosphoprotein-related | 2.63 |
| At3g20240 | mitochondrial substrate carrier family protein | 2.63 |
| At1g16000 | expressed protein | 2.62 |
| At3g18850 | phospholipid/glycerol acyltransferase family protein | 2.62 |
| At2g04570 | GDSL-motif lipase/hydrolase family protein | 2.61 |
| At2g20940 | expressed protein | 2.61 |
| At5g54670 | kinesin-like protein C (KATC) | 2.61 |
| At3g07410 | Ras-related GTP-binding family protein | 2.61 |
| At2g39920 | acid phosphatase class B family protein | 2.60 |
| At3g62860 | esterase/lipase/thioesterase family protein | 2.60 |
| At2g44640 | expressed protein | 2.60 |
| At4g12600 | ribosomal protein L7Ae/L30e/S12e/Gadd45 family protein | 2.60 |
| At5g62710 | leucine-rich repeat family protein/protein kinase family protein | 2.60 |
| At5g01370 | expressed protein | 2.60 |
| At5g01910 | hypothetical protein | 2.59 |
| At5g14700 | cinnamoyl-CoA reductase-related | 2.59 |
| At5g25590 | expressed protein | 2.59 |
| At5g46280 | DNA replication licensing factor, putative | 2.59 |
| At5g40330 | myb family transcription factor | 2.58 |
| At3g57150 | dyskerin, putative/nucleolar protein NAP57, putative | 2.57 |
| At1g61890 | MATE efflux family protein | 2.57 |
| At5g14920 | gibberellin-regulated family protein | 2.57 |
| At2g42110 | expressed protein | 2.57 |
| At1g14620 | expressed protein | 2.57 |
| At1g05440 | expressed protein | 2.57 |
| At1g29270 | expressed protein | 2.57 |
| At1g06620 | 2-oxoglutarate-dependent dioxygenase, putative | 2.57 |
| At1g69690 | TCP family transcription factor, putative | 2.57 |
| At1g07370 | proliferating cell nuclear antigen 1 (PCNA1) | 2.56 |
| At5g07180 | leucine-rich repeat family protein/protein kinase family protein | 2.56 |
| At2g16780 | WD-40 repeat protein (MSI2) | 2.56 |
| At5g66230 | expressed protein | 2.56 |
| At4g39260 | glycine-rich RNA-binding protein 8 (GRP8) (CCR1) | 2.56 |
| At1g11820 | glycosyl hydrolase family 17 protein | 2.56 |
| At4g16140 | proline-rich family protein | 2.55 |
| At3g13650 | disease resistance response protein-related/dirigent protein-related | 2.55 |
| At3g43960 | cysteine proteinase, putative | 2.55 |
| At5g13520 | peptidase M1 family protein | 2.55 |
| At4g16515 | expressed protein | 2.54 |
| At3g28900 | 60S ribosomal protein L34 (RPL34C) | 2.54 |
| At4g22120 | early-responsive to dehydration protein-related/ERD protein-related | 2.54 |
| At1g30820 | CTP synthase, putative/UTP--ammonia ligase, putative | 2.54 |
| At2g05790 | glycosyl hydrolase family 17 protein | 2.53 |
| At1g13280 | allene oxide cyclase family protein | 2.53 |
| At5g11510 | myb family transcription factor (MYB3R4) | 2.52 |
| At1g02200 | CER1 protein | 2.52 |
| At4g20430 | subtilase family protein | 2.51 |
| At1g32640 | basic helix-loop-helix (bHLH) protein (RAP-1) | 2.51 |
| At1g70210 | cyclin delta-1 (CYCD1) | 2.51 |
| At1g55020 | lipoxygenase (LOX1) | 2.51 |
| At2g25210 | 60S ribosomal protein L39 (RPL39A) | 2.51 |
| At4g34200 | D-3-phosphoglycerate dehydrogenase, putative/3-PGDH, putative | 2.51 |
| At1g71260 | expressed protein | 2.51 |
| At1g28230 | purine permease (PUP1) | 2.51 |
| At5g65080 | MADS-box family protein | 2.50 |
| At5g07990 | flavonoid 3′-monooxygenase/flavonoid 3′-hydroxylase (F3′H)/ | 2.50 |
| | cytochrome P450 75B1 (CYP75B1)/transparent testa 7 protein (TT7) | |
| At5g48490 | protease inhibitor/seed storage/lipid transfer protein (LTP) family protein | 2.50 |
| At5g07230 | protease inhibitor/seed storage/lipid transfer protein (LTP) family protein | 2.49 |
| At4g01080 | expressed protein | 2.49 |
| At3g50280 | transferase family protein | 2.49 |
| At1g63710 | cytochrome P450, putative | 2.49 |
| At1g73870 | zinc finger (B-box type) family protein | 2.48 |
| At5g67150 | transferase family protein | 2.48 |
| At5g03390 | expressed protein | 2.48 |
| At1g28530 | expressed protein | 2.48 |
| At5g63810 | beta-galactosidase, putative/lactase, putative | 2.48 |
| At3g54400 | aspartyl protease family protein | 2.48 |
| At3g03630 | cysteine synthase, chloroplast, putative/O-acetylserine (thiol)-lyase, | 2.48 |
| | putative/O-acetylserine sulfhydrylase, putative | |
| At4g09510 | beta-fructofuranosidase, putative/invertase, putative/saccharase, | 2.48 |
| | putative/beta-fructosidase, putative | |
| At4g28680 | tyrosine decarboxylase, putative | 2.48 |
| At5g09300 | 2-oxoisovalerate dehydrogenase, putative/3-methyl-2-oxobutanoate | 2.47 |
| | dehydrogenase, putative/branched-chain alpha-keto acid | |
| | dehydrogenase E1 alpha subunit, putative | |
| At2g38400 | alanine--glyoxylate aminotransferase, putative/beta-alanine-pyruvate | 2.47 |
| | aminotransferase, putative/AGT, putative | |
| At3g05810 | expressed protein | 2.47 |
| At2g38810 | histone H2A, putative | 2.47 |
| At2g36870 | xyloglucan:xyloglucosyl transferase, putative/xyloglucan | 2.47 |
| | endotransglycosylase, putative/endo-xyloglucan transferase, putative | |
| At2g20450 | 60S ribosomal protein L14 (RPL14A) | 2.46 |
| At5g39860 | bHLH protein | 2.46 |
| At2g36400 | expressed protein | 2.46 |
| At3g51290 | proline-rich family protein | 2.46 |
| At5g10830 | embryo-abundant protein-related | 2.46 |
| At4g35730 | expressed protein | 2.46 |
| At4g15640 | expressed protein | 2.46 |
| At2g37690 | phosphoribosylaminoimidazole carboxylase family protein/AIR | 2.46 |
| | carboxylase family protein | |
| At4g39980 | 2-dehydro-3-deoxyphosphoheptonate aldolase 1/3-deoxy-D-arabino- | 2.46 |
| | heptulosonate 7-phosphate synthase 1/DAHP synthetase 1 (DHS1) | |
| At1g72500 | inter-alpha-trypsin inhibitor heavy chain-related | 2.46 |
| At5g66440 | expressed protein | 2.46 |
| At1g68500 | expressed protein | 2.45 |
| At3g13470 | chaperonin, putative | 2.45 |
| At4g34290 | SWIB complex BAF60b domain-containing protein | 2.45 |
| At3g11900 | amino acid transporter family protein | 2.45 |
| At3g52170 | expressed protein | 2.45 |
| At4g02060 | prolifera protein (PRL)/DNA replication licensing factor Mcm7 (MCM7) | 2.45 |
| At1g53140 | dynamin family protein | 2.45 |
| At1g48630 | guanine nucleotide-binding family protein/activated protein kinase C | 2.45 |
| | receptor, putative/RACK, putative | |
| At3g05640 | protein phosphatase 2C, putative/PP2C, putative | 2.44 |
| At1g30530 | UDP-glucoronosyl/UDP-glucosyl transferase family protein | 2.44 |
| At2g28410 | expressed protein | 2.44 |
| At3g53370 | DNA-binding S1FA family protein | 2.44 |
| At4g15140 | expressed protein | 2.44 |
| At3g61490 | glycoside hydrolase family 28 protein/polygalacturonase (pectinase) | 2.43 |
| | family protein | |
| At5g46700 | senescence-associated protein, putative | 2.43 |
| At4g25260 | invertase/pectin methylesterase inhibitor family protein | 2.43 |
| At2g20670 | expressed protein | 2.43 |
| At3g17170 | ribosomal protein S6 family protein (RFC3) | 2.43 |
| At2g37340 | splicing factor RSZ33 (RSZ33) | 2.43 |
| At5g50370 | adenylate kinase, putative | 2.43 |
| At3g05890 | hydrophobic protein (RCI2B)/low temperature and salt responsive | 2.43 |
| | protein (LTI6B) | |
| At5g57180 | expressed protein | 2.43 |
| At4g15620 | integral membrane family protein | 2.42 |
| At1g10980 | expressed protein | 2.42 |
| At1g05620 | inosine-uridine preferring nucleoside hydrolase family protein | 2.42 |
| At3g14760 | expressed protein | 2.42 |
| At5g19730 | pectinesterase family protein | 2.42 |
| At5g27450 | mevalonate kinase (MK) | 2.42 |
| At1g63100 | scarecrow transcription factor family protein | 2.41 |
| At2g42740 | 60S ribosomal protein L11 (RPL11A) | 2.41 |
| At4g10390 | protein kinase family protein | 2.41 |
| At4g39860 | expressed protein | 2.41 |
| At3g11520 | cyclin, putative (CYC2) | 2.41 |
| At3g25190 | nodulin, putative | 2.41 |
| At2g37300 | expressed protein | 2.41 |
| At3g59650 | mitochondrial ribosomal protein L51/S25/CI-B8 family protein | 2.41 |
| At4g25150 | acid phosphatase, putative | 2.41 |
| At5g56500 | chaperonin, putative | 2.40 |
| At5g19090 | heavy-metal-associated domain-containing protein | 2.40 |
| At5g28490 | expressed protein | 2.40 |
| At1g65370 | meprin and TRAF homology domain-containing protein/MATH domain- | 2.40 |
| | containing protein | |
| At5g12080 | mechanosensitive ion channel domain-containing protein/MS ion | 2.40 |
| | channel domain-containing protein | |
| At1g31700 | copper amine oxidase, putative | 2.40 |
| At3g61820 | aspartyl protease family protein | 2.39 |
| At1g14980 | 10 kDa chaperonin (CPN10) | 2.39 |
| At2g43550 | trypsin inhibitor, putative | 2.39 |
| At3g27220 | kelch repeat-containing protein | 2.38 |
| At3g02110 | serine carboxypeptidase S10 family protein | 2.38 |
| At4g30800 | 40S ribosomal protein S11 (RPS11B) | 2.38 |
| At3g11120 | 60S ribosomal protein L41 (RPL41C) /// 60S ribosomal protein L41 | 2.38 |
| | (RPL41C) | |
| At5g12110 | elongation factor 1B alpha-subunit 1 (eEF1Balpha1) | 2.38 |
| At3g06220 | transcriptional factor B3 family protein | 2.38 |
| At5g06870 | polygalacturonase inhibiting protein 2 (PGIP2) | 2.38 |
| At3g61100 | expressed protein | 2.38 |
| At5g15530 | biotin carboxyl carrier protein 2 (BCCP2) | 2.38 |
| At1g11080 | serine carboxypeptidase S10 family protein | 2.37 |
| At4g26780 | co-chaperone grpE family protein | 2.37 |
| At3g58650 | expressed protein | 2.37 |
| At5g03230 | expressed protein | 2.37 |
| At5g51910 | TCP family transcription factor, putative | 2.37 |
| At4g15490 | UDP-glucoronosyl/UDP-glucosyl transferase family protein | 2.37 |
| At1g30600 | subtilase family protein | 2.37 |
| At5g02430 | WD-40 repeat family protein | 2.37 |
| At3g47500 | Dof-type zinc finger domain-containing protein | 2.37 |
| At3g16950 | dihydrolipoamide dehydrogenase 1, plastidic/lipoamide dehydrogenase | 2.36 |
| | 1 (PTLPD1) | |
| At4g01730 | zinc finger (DHHC type) family protein | 2.36 |
| At3g23410 | alcohol oxidase-related | 2.36 |
| At5g15520 | 40S ribosomal protein S19 (RPS19B) | 2.36 |
| At5g45040 | cytochrome c6 (ATC6) | 2.35 |
| At1g18330 | myb family transcription factor | 2.35 |
| At2g46640 | hypothetical protein | 2.35 |
| At4g10955 | lipase class 3 family protein | 2.35 |
| At5g15780 | pollen Ole e 1 allergen and extensin family protein | 2.35 |
| At5g38130 | transferase family protein | 2.35 |
| At1g06640 | 2-oxoglutarate-dependent dioxygenase, putative | 2.35 |
| At4g18970 | GDSL-motif lipase/hydrolase family protein | 2.35 |
| At2g05990 | enoyl-(acyl-carrier protein) reductase (NADH), chloroplast, putative/ | 2.34 |
| | NADH-dependent enoyl-ACP reductase, putative | |
| At5g22440 | 60S ribosomal protein L10A (RPL10aC) | 2.34 |
| At3g17120 | expressed protein | 2.34 |
| At2g43010 | phytochrome-interacting factor 4 (PIF4)/basic helix-loop-helix protein 9 | 2.34 |
| | (bHLH9)/short under red-light 2 (SRL2) | |
| At4g34138 | UDP-glucoronosyl/UDP-glucosyl transferase family protein | 2.34 |
| At1g23790 | expressed protein | 2.33 |
| At5g01820 | CBL-interacting protein kinase 14 (CIPK14) | 2.33 |
| At5g43810 | pinhead protein (PINHEAD)/zwille protein (ZWILLE) | 2.33 |
| At5g07280 | leucine-rich repeat protein kinase, putative/extra sporogenous cells | 2.33 |
| | (ESP) | |
| At4g37320 | cytochrome P450 family protein | 2.33 |
| At1g18710 | myb family transcription factor (MYB47) | 2.33 |
| At3g44320 | nitrilase 3 (NIT3) | 2.32 |
| At3g54560 | histone H2A.F/Z | 2.32 |
| At1g19540 | isoflavone reductase, putative | 2.32 |
| At3g53380 | lectin protein kinase family protein | 2.32 |
| At2g36350 | protein kinase, putative | 2.32 |
| At2g37770 | aldo/keto reductase family protein | 2.31 |
| At3g07390 | auxin-responsive protein/auxin-induced protein (AIR12) | 2.31 |
| At4g30660 | low temperature and salt responsive protein, putative | 2.31 |
| At2g17630 | phosphoserine aminotransferase, putative | 2.31 |
| At5g39320 | UDP-glucose 6-dehydrogenase, putative | 2.31 |
| At1g29250 | expressed protein | 2.31 |
| At3g51450 | strictosidine synthase family protein | 2.31 |
| At4g37870 | phosphoenolpyruvate carboxykinase (ATP), putative/PEP | 2.31 |
| | carboxykinase, putative/PEPCK, putative | |
| At3g17160 | expressed protein | 2.30 |
| At1g72630 | expressed protein | 2.30 |
| At2g34190 | xanthine/uracil permease family protein | 2.30 |
| At2g23180 | cytochrome P450, putative | 2.30 |
| At5g57660 | zinc finger (B-box type) family protein | 2.30 |
| At2g15090 | fatty acid elongase, putative | 2.30 |
| At4g34710 | arginine decarboxylase 2 (SPE2) | 2.30 |
| At1g23410 | ubiquitin extension protein, putative/40S ribosomal protein S27A | 2.30 |
| | (RPS27aA) | |
| At1g44970 | peroxidase, putative | 2.29 |
| At1g35290 | thioesterase family protein | 2.29 |
| At1g75580 | auxin-responsive protein, putative | 2.29 |
| At1g72140 | proton-dependent oligopeptide transport (POT) family protein | 2.29 |
| At1g04520 | 33 kDa secretory protein-related | 2.29 |
| At1g68650 | expressed protein | 2.29 |
| At1g22550 | proton-dependent oligopeptide transport (POT) family protein | 2.29 |
| At2g04032 | metal transporter, putative (ZIP7) | 2.29 |
| At1g66850 | protease inhibitor/seed storage/lipid transfer protein (LTP) family protein | 2.28 |
| At3g48460 | GDSL-motif lipase/hydrolase family protein | 2.28 |
| At3g10870 | hydrolase, alpha/beta fold family protein | 2.28 |
| At4g15190 | — | 2.28 |
| At5g23300 | dihydroorotate dehydrogenase, mitochondrial/dihydroorotate oxidase/ | 2.28 |
| | DHOdehase (PYRD) | |
| At1g64450 | proline-rich family protein | 2.28 |
| At1g16780 | vacuolar-type H+-translocating inorganic pyrophosphatase, putative | 2.28 |
| At4g28230 | expressed protein | 2.27 |
| At1g61610 | S-locus lectin protein kinase family protein | 2.27 |
| At4g03190 | F-box family protein (FBL18) | 2.27 |
| At2g26180 | calmodulin-binding family protein | 2.27 |
| At5g47700 | 60S acidic ribosomal protein P1 (RPP1C) | 2.26 |
| At1g76930 | proline-rich extensin-like family protein | 2.26 |
| At2g28620 | kinesin motor protein-related | 2.26 |
| At3g45450 | Clp amino terminal domain-containing protein /// Clp amino terminal | 2.26 |
| | domain-containing protein | |
| At3g14220 | GDSL-motif lipase/hydrolase family protein | 2.26 |
| At3g25900 | homocysteine S-methyltransferase 1 (HMT-1) | 2.26 |
| At2g30040 | protein kinase family protein | 2.26 |
| At1g62660 | beta-fructosidase (BFRUCT3)/beta-fructofuranosidase/invertase, | 2.26 |
| | vacuolar | |
| At4g26670 | mitochondrial import inner membrane translocase subunit | 2.25 |
| | Tim17/Tim22/Tim23 family protein | |
| At4g15210 | beta-amylase (BMY1)/1,4-alpha-D-glucan maltohydrolase | 2.25 |
| At4g20130 | ribulose-1,5 bisphosphate carboxylase/oxygenase large subunit N- | 2.25 |
| | methyltransferase-related | |
| At3g10610 | 40S ribosomal protein S17 (RPS17C) | 2.25 |
| At1g04820 | tubulin alpha-2/alpha-4 chain (TUA4) | 2.24 |
| At4g20930 | 3-hydroxyisobutyrate dehydrogenase, putative | 2.24 |
| At3g23940 | dehydratase family | 2.24 |
| At1g52720 | expressed protein | 2.24 |
| At2g36885 | expressed protein | 2.24 |
| At4g34400 | transcriptional factor B3 family protein | 2.24 |
| At2g29570 | proliferating cell nuclear antigen 2 (PCNA2) | 2.24 |
| At4g27010 | expressed protein | 2.24 |
| At2g41540 | NAD-dependent glycerol-3-phosphate dehydrogenase family protein | 2.23 |
| At2g38940 | phosphate transporter (PT2) | 2.23 |
| At2g22900 | galactosyl transferase GMA12/MNN10 family protein | 2.23 |
| At5g52860 | ABC transporter family protein | 2.23 |
| At2g45190 | axial regulator YABBY1 (YABBY1)/abnormal floral organs protein | 2.23 |
| | (AFO)/filamentous flower protein (FIL) | |
| At3g23295 | expressed protein | 2.23 |
| At3g23290 | expressed protein | 2.23 |
| At5g62360 | invertase/pectin methylesterase inhibitor family protein | 2.23 |
| At2g31160 | expressed protein | 2.22 |
| At5g42900 | expressed protein | 2.22 |
| At4g13170 | 60S ribosomal protein L13A (RPL13aC) | 2.22 |
| At2g14890 | arabinogalactan-protein (AGP9) | 2.22 |
| At2g33620 | DNA-binding family protein/AT-hook protein 1 (AHP1) | 2.22 |
| At2g35120 | glycine cleavage system H protein, mitochondrial, putative | 2.22 |
| At1g80110 | expressed protein | 2.22 |
| At4g30650 | hydrophobic protein, putative/low temperature and salt responsive | 2.21 |
| | protein, putative | |
| At4g16730 | terpene synthase/cyclase family protein | 2.21 |
| At3g52130 | protease inhibitor/seed storage/lipid transfer protein (LTP) family protein | 2.21 |
| At1g63240 | expressed protein | 2.21 |
| At1g74420 | xyloglucan fucosyltransferase, putative (FUT3) | 2.21 |
| At2g16440 | DNA replication licensing factor, putative | 2.21 |
| At4g26790 | GDSL-motif lipase/hydrolase family protein | 2.21 |
| At2g20490 | nucleolar RNA-binding Nop10p family protein | 2.20 |
| At3g57830 | leucine-rich repeat transmembrane protein kinase, putative | 2.20 |
| At4g25240 | multi-copper oxidase type I family protein | 2.20 |
| At3g20015 | aspartyl protease family protein | 2.20 |
| At5g06550 | transcription factor jumonji (jmjC) domain-containing protein | 2.20 |
| At5g62690 | tubulin beta-2/beta-3 chain (TUB2) /// tubulin beta-2/beta-3 chain (TUB2) | 2.20 |
| At2g19780 | leucine-rich repeat family protein/extensin family protein | 2.20 |
| At5g50580 | SUMO activating enzyme, putative /// SUMO activating enzyme, putative | 2.20 |
| At5g47330 | palmitoyl protein thioesterase family protein | 2.20 |
| At2g30500 | kinase interacting family protein | 2.19 |
| At5g53210 | basic helix-loop-helix (bHLH) family protein | 2.19 |
| At5g05600 | oxidoreductase, 2OG-Fe(II) oxygenase family protein | 2.19 |
| At1g49320 | BURP domain-containing protein | 2.19 |
| At1g65890 | acyl-activating enzyme 12 (AAE12) | 2.19 |
| At5g23400 | disease resistance family protein/LRR family protein | 2.19 |
| At5g48820 | kip-related protein 3 (KRP3)/cyclin-dependent kinase inhibitor 3 (ICK3) | 2.19 |
| At5g11550 | expressed protein | 2.19 |
| At2g37420 | kinesin motor protein-related | 2.18 |
| At2g33370 | 60S ribosomal protein L23 (RPL23B) | 2.18 |
| At1g21550 | calcium-binding protein, putative | 2.18 |
| At5g01075 | expressed protein | 2.18 |
| At1g53070 | legume lectin family protein | 2.18 |
| At5g15120 | expressed protein | 2.18 |
| At2g37790 | aldo/keto reductase family protein | 2.18 |
| At5g54970 | expressed protein | 2.18 |
| At3g29230 | pentatricopeptide (PPR) repeat-containing protein | 2.18 |
| At3g18510 | expressed protein | 2.18 |
| At5g64860 | 4-alpha-glucanotransferase, putative/disproportionating enzyme, | 2.18 |
| | putative | |
| At2g29730 | UDP-glucoronosyl/UDP-glucosyl transferase family protein /// UDP- | 2.18 |
| | glucoronosyl/UDP-glucosyl transferase family protein | |
| At1g33811 | GDSL-motif lipase/hydrolase family protein | 2.18 |
| At1g44835 | YbaK/prolyl-tRNA synthetase family protein | 2.17 |
| At4g17190 | farnesyl pyrophosphate synthetase 2 (FPS2)/FPP synthetase 2 | 2.17 |
| At5g23740 | 40S ribosomal protein S11 (RPS11C) | 2.17 |
| At1g77120 | alcohol dehydrogenase (ADH) | 2.17 |
| At2g21130 | peptidyl-prolyl cis-trans isomerase/cyclophilin (CYP2)/rotamase | 2.17 |
| At4g12880 | plastocyanin-like domain-containing protein | 2.17 |
| At4g15000 | 60S ribosomal protein L27 (RPL27C) | 2.17 |
| At4g05190 | kinesin-like protein A, putative | 2.16 |
| At3g03130 | expressed protein | 2.16 |
| At4g12970 | expressed protein | 2.16 |
| At5g47600 | heat shock protein-related | 2.16 |
| At5g47455 | expressed protein | 2.16 |
| At5g26220 | ChaC-like family protein | 2.16 |
| At3g26290 | cytochrome P450 71B26, putative (CYP71B26) | 2.16 |
| At4g17240 | expressed protein | 2.15 |
| At1g23100 | 10 kDa chaperonin, putative | 2.15 |
| At4g04190 | expressed protein | 2.15 |
| At5g25490 | zinc finger (Ran-binding) family protein | 2.15 |
| At5g59480 | haloacid dehalogenase-like hydrolase family protein | 2.15 |
| At1g78260 | RNA recognition motif (RRM)-containing protein | 2.15 |
| At2g32650 | expressed protein /// expressed protein | 2.15 |
| At2g06050 | 12-oxophytodienoate reductase (OPR3)/delayed dehiscence1 (DDE1) | 2.15 |
| At1g16530 | LOB domain protein 3/lateral organ boundaries domain protein 3 | 2.15 |
| | (LBD3) | |
| At5g56940 | ribosomal protein S16 family protein | 2.15 |
| At2g44450 | glycosyl hydrolase family 1 protein | 2.15 |
| At2g29890 | villin 1 (VLN1) | 2.14 |
| At3g51400 | expressed protein | 2.14 |
| At2g37380 | expressed protein | 2.14 |
| At4g38890 | dihydrouridine synthase family protein | 2.14 |
| At4g21910 | MATE efflux family protein | 2.14 |
| At1g65920 | regulator of chromosome condensation (RCC1) family protein/zinc | 2.14 |
| | finger protein-related | |
| At1g20590 | cyclin, putative /// cyclin, putative | 2.14 |
| At1g79530 | glyceraldehyde 3-phosphate dehydrogenase, cytosolic, putative/NAD- | 2.14 |
| | dependent glyceraldehyde-3-phosphate dehydrogenase, putative /// | |
| | glyceraldehyde 3-phosphate dehydrogenase, cytosolic, putative/NAD- | |
| | dependent glyceraldehyde-3-phosphate dehydrogenase, pu | |
| At1g20930 | cell division control protein, putative | 2.14 |
| At1g35260 | Bet v I allergen family protein | 2.14 |
| At5g02280 | synbindin, putative | 2.14 |
| At5g10400 | histone H3 | 2.14 |
| At4g28950 | Rac-like GTP-binding protein (ARAC7) | 2.14 |
| At4g18280 | glycine-rich cell wall protein-related | 2.14 |
| At4g23990 | cellulose synthase family protein | 2.14 |
| At5g08400 | expressed protein | 2.14 |
| At1g16790 | ribosomal protein-related | 2.13 |
| At5g65890 | ACT domain-containing protein (ACR1) | 2.13 |
| At5g54160 | quercetin 3-O-methyltransferase 1/flavonol 3-O-methyltransferase 1/ | 2.13 |
| | caffeic acid/5-hydroxyferulic acid O-methyltransferase (OMT1) | |
| At5g27330 | expressed protein | 2.13 |
| At5g67280 | leucine-rich repeat transmembrane protein kinase, putative | 2.13 |
| At5g04620 | aminotransferase class I and II family protein | 2.13 |
| At1g49760 | polyadenylate-binding protein, putative/PABP, putative | 2.13 |
| At3g60360 | expressed protein | 2.13 |
| At4g02150 | importin alpha-2 subunit | 2.13 |
| At5g26665 | pectinacetylesterase, putative | 2.13 |
| At3g62700 | glutathione-conjugate transporter, putative | 2.13 |
| At3g55280 | 60S ribosomal protein L23A (RPL23aB) | 2.12 |
| At3g13160 | pentatricopeptide (PPR) repeat-containing protein | 2.12 |
| At3g55310 | short-chain dehydrogenase/reductase (SDR) family protein | 2.12 |
| At5g60930 | chromosome-associated kinesin, putative | 2.12 |
| At1g58430 | GDSL-motif lipase/hydrolase family protein | 2.12 |
| At2g13610 | ABC transporter family protein | 2.11 |
| At1g05210 | expressed protein | 2.11 |
| At4g13210 | pectate lyase family protein | 2.11 |
| At2g20515 | expressed protein | 2.11 |
| At5g61290 | flavin-containing monooxygenase family protein/FMO family protein | 2.11 |
| At2g38820 | expressed protein | 2.11 |
| At1g16350 | inosine-5′-monophosphate dehydrogenase, putative | 2.11 |
| At5g23860 | tubulin beta-8 chain (TUB8) (TUBB8) | 2.11 |
| At3g04920 | 40S ribosomal protein S24 (RPS24A) | 2.11 |
| At1g49560 | myb family transcription factor | 2.11 |
| At2g19810 | zinc finger (CCCH-type) family protein | 2.11 |
| At4g14270 | expressed protein | 2.11 |
| At3g27360 | histone H3 | 2.10 |
| At2g19720 | 40S ribosomal protein S15A (RPS15aB) | 2.10 |
| At3g13510 | expressed protein | 2.10 |
| At3g56130 | biotin/lipoyl attachment domain-containing protein | 2.10 |
| At3g07770 | heat shock protein-related | 2.10 |
| At1g78170 | expressed protein | 2.10 |
| At2g40010 | 60S acidic ribosomal protein P0 (RPP0A) | 2.10 |
| At4g17480 | palmitoyl protein thioesterase family protein | 2.10 |
| At5g43060 | cysteine proteinase, putative/thiol protease, putative | 2.10 |
| At3g54260 | expressed protein | 2.10 |
| At5g60670 | 60S ribosomal protein L12 (RPL12C) | 2.10 |
| At3g17840 | leucine-rich repeat transmembrane protein kinase, putative | 2.09 |
| At2g18400 | ribosomal protein L6 family protein | 2.09 |
| At1g14180 | expressed protein | 2.09 |
| At5g63980 | 3′(2′),5′-bisphosphate nucleotidase/inositol polyphosphate 1- | 2.09 |
| | phosphatase/FIERY1 protein (FRY1) (SAL1) | |
| At2g05540 | glycine-rich protein | 2.09 |
| At2g22240 | inositol-3-phosphate synthase isozyme 2/myo-inositol-1-phosphate | 2.09 |
| | synthase 2/MI-1-P synthase 2/IPS 2 | |
| At5g02940 | expressed protein | 2.09 |
| At1g72230 | plastocyanin-like domain-containing protein | 2.09 |
| At3g51090 | expressed protein | 2.09 |
| At3g28860 | multidrug resistance P-glycoprotein, putative | 2.09 |
| At2g36380 | ABC transporter family protein | 2.09 |
| At3g16490 | calmodulin-binding family protein | 2.09 |
| At4g39200 | 40S ribosomal protein S25 (RPS25E) | 2.09 |
| At2g40360 | transducin family protein/WD-40 repeat family protein | 2.09 |
| At5g57290 | 60S acidic ribosomal protein P3 (RPP3B) | 2.09 |
| At2g32765 | small ubiquitin-like modifier 5 (SUMO) | 2.09 |
| At3g14200 | DNAJ heat shock N-terminal domain-containing protein | 2.08 |
| At5g08610 | DEAD box RNA helicase (RH26) | 2.08 |
| At3g52580 | 40S ribosomal protein S14 (RPS14C) | 2.08 |
| At3g18080 | glycosyl hydrolase family 1 protein | 2.08 |
| At3g53740 | 60S ribosomal protein L36 (RPL36B) | 2.08 |
| At4g05390 | ferredoxin--NADP(+) reductase, putative/adrenodoxin reductase, | 2.08 |
| | putative | |
| At1g73177 | expressed protein | 2.08 |
| At5g60300 | lectin protein kinase family protein | 2.08 |
| At2g22610 | kinesin motor protein-related | 2.08 |
| At2g16630 | proline-rich family protein | 2.08 |
| At5g57410 | expressed protein | 2.08 |
| At4g31700 | 40S ribosomal protein S6 (RPS6A) | 2.08 |
| At2g45970 | cytochrome P450, putative | 2.08 |
| At3g51740 | leucine-rich repeat transmembrane protein kinase, putative | 2.08 |
| At1g53520 | chalcone-flavanone isomerase-related | 2.08 |
| At1g51100 | expressed protein | 2.08 |
| At5g17490 | gibberellin response modulator, putative | 2.07 |
| At5g57630 | CBL-interacting protein kinase 21, putative (CIPK21) | 2.07 |
| At4g14080 | glycosyl hydrolase family 17 protein/anther-specific protein (A6) | 2.07 |
| At4g28590 | expressed protein | 2.07 |
| At3g09460 | expressed protein | 2.07 |
| At2g37190 | 60S ribosomal protein L12 (RPL12A) | 2.07 |
| At2g43020 | amine oxidase family protein | 2.07 |
| At5g27550 | kinesin motor protein-related | 2.07 |
| At1g02180 | ferredoxin-related | 2.07 |
| At2g35605 | SWIB complex BAF60b domain-containing protein | 2.06 |
| At4g26230 | 60S ribosomal protein L31 (RPL31B) | 2.06 |
| At1g50110 | branched-chain amino acid aminotransferase 6 (BCAT6) | 2.06 |
| At4g05400 | expressed protein | 2.06 |
| At2g27710 | 60S acidic ribosomal protein P2 (RPP2B) | 2.06 |
| At5g28060 | 40S ribosomal protein S24 (RPS24B) | 2.06 |
| At3g04000 | short-chain dehydrogenase/reductase (SDR) family protein | 2.06 |
| At1g03360 | exonuclease family protein | 2.06 |
| At3g49910 | 60S ribosomal protein L26 (RPL26A) | 2.06 |
| At3g02790 | zinc finger (C2H2 type) family protein | 2.06 |
| At4g10380 | major intrinsic family protein/MIP family protein | 2.06 |
| At3g12270 | protein arginine N-methyltransferase family protein | 2.05 |
| At4g01850 | S-adenosylmethionine synthetase 2 (SAM2) | 2.05 |
| At5g51100 | superoxide dismutase (Fe), putative | 2.05 |
| At1g64660 | Cys/Met metabolism pyridoxal-phosphate-dependent enzyme family | 2.05 |
| | protein | |
| At5g62210 | embryo-specific protein-related | 2.05 |
| At1g29980 | expressed protein | 2.05 |
| At1g61120 | terpene synthase/cyclase family protein | 2.05 |
| At3g52550 | hypothetical protein | 2.05 |
| At2g29440 | glutathione S-transferase, putative | 2.05 |
| At2g43460 | 60S ribosomal protein L38 (RPL38A) | 2.05 |
| At3g49260 | calmodulin-binding family protein | 2.05 |
| At1g64170 | cation/hydrogen exchanger, putative (CHX16) | 2.05 |
| At1g35180 | expressed protein /// expressed protein | 2.05 |
| At5g24780 | vegetative storage protein 2 (VSP2) | 2.05 |
| At1g52930 | brix domain-containing protein | 2.05 |
| At2g03090 | expansin, putative (EXP15) | 2.05 |
| At2g18990 | expressed protein | 2.05 |
| At4g29510 | protein arginine N-methyltransferase, putative | 2.04 |
| At1g01100 | 60S acidic ribosomal protein P1 (RPP1A) | 2.04 |
| At2g24440 | expressed protein | 2.04 |
| At2g22170 | lipid-associated family protein | 2.04 |
| At4g34850 | chalcone and stilbene synthase family protein | 2.04 |
| At5g54530 | expressed protein | 2.04 |
| At3g44010 | 40S ribosomal protein S29 (RPS29A) | 2.04 |
| At1g19570 | dehydroascorbate reductase, putative | 2.04 |
| At2g32060 | 40S ribosomal protein S12 (RPS12C) | 2.04 |
| At2g19170 | subtilase family protein | 2.04 |
| At5g40610 | glycerol-3-phosphate dehydrogenase (NAD+)/GPDH | 2.04 |
| At3g11670 | digalactosyldiacylglycerol synthase 1 (DGD1)/MGDG:MGDG | 2.04 |
| | galactosyltransferase/galactolipid galactosyltransferase | |
| At2g22330 | cytochrome P450, putative | 2.04 |
| At4g34670 | 40S ribosomal protein S3A (RPS3aB) | 2.04 |
| At5g48880 | acetyl-CoA C-acyltransferase 1/3-ketoacyl-CoA thiolase 1 (PKT1) | 2.04 |
| At3g06840 | expressed protein | 2.03 |
| At4g26660 | expressed protein | 2.03 |
| At5g09770 | ribosomal protein L17 family protein | 2.03 |
| At4g17610 | tRNA/rRNA methyltransferase (SpoU) family protein | 2.03 |
| At5g61170 | 40S ribosomal protein S19 (RPS19C) | 2.03 |
| At1g18140 | laccase family protein/diphenol oxidase family protein | 2.03 |
| At1g51610 | cation efflux family protein/metal tolerance protein, putative (MTPc4) | 2.03 |
| At2g40435 | expressed protein | 2.02 |
| At3g44970 | cytochrome P450 family protein | 2.02 |
| At5g59350 | expressed protein | 2.02 |
| At5g65870 | phytosulfokines 5 (PSK5) | 2.02 |
| At5g62290 | nucleotide-sensitive chloride conductance regulator (ICIn) family protein | 2.02 |
| At1g62510 | protease inhibitor/seed storage/lipid transfer protein (LTP) family protein | 2.02 |
| At3g47750 | ABC transporter family protein | 2.02 |
| At5g07090 | 40S ribosomal protein S4 (RPS4B) | 2.02 |
| At3g22740 | homocysteine S-methyltransferase 3 (HMT-3) | 2.01 |
| At3g61840 | expressed protein | 2.01 |
| At4g37150 | esterase, putative | 2.01 |
| At4g18910 | aquaglyceroporin/NOD26-like major intrinsic protein 2 (NLM2) | 2.01 |
| At3g02080 | 40S ribosomal protein S19 (RPS19A) | 2.01 |
| At1g07790 | histone H2B, putative | 2.01 |
| At2g37390 | heavy-metal-associated domain-containing protein | 2.01 |
| At1g03110 | transducin family protein/WD-40 repeat family protein | 2.01 |
| At1g23800 | aldehyde dehydrogenase, mitochondrial (ALDH3) | 2.01 |
| At2g39250 | AP2 domain-containing transcription factor, putative | 2.01 |
| At2g25270 | expressed protein | 2.01 |
| At5g48760 | 60S ribosomal protein L13A (RPL13aD) | 2.01 |
| At3g21740 | expressed protein | 2.01 |
| At4g33490 | nucellin protein, putative | 2.01 |
| At3g60490 | AP2 domain-containing transcription factor TINY, putative | 2.01 |
| At2g32590 | barren family protein | 2.01 |
| ccb382 | — | 2.01 |
| At2g41340 | eukaryotic rpb5 RNA polymerase subunit family protein | 2.01 |
| At2g36170 | ubiquitin extension protein 2 (UBQ2)/60S ribosomal protein L40 | 2.01 |
| | (RPL40A) | |
| At3g54630 | expressed protein | 2.01 |
| At5g01790 | expressed protein | 2.01 |
| At1g63160 | replication factor C 40 kDa, putative | 2.00 |
| At5g27930 | protein phosphatase 2C, putative/PP2C, putative | 2.00 |
| At1g33940 | hypothetical protein | 2.00 |
| At5g66750 | SNF2 domain-containing protein/helicase domain-containing protein | 2.00 |
| At4g29390 | 40S ribosomal protein S30 (RPS30B) | 2.00 |
| At1g79560 | FtsH protease, putative | 2.00 |
| At3g46560 | mitochondrial import inner membrane translocase (TIM9) | 2.00 |
| At2g44120 | 60S ribosomal protein L7 (RPL7C) | 2.00 |
| At4g03110 | RNA-binding protein, putative | −2.00 |
| At3g02800 | tyrosine specific protein phosphatase family protein | −2.00 |
| At2g24280 | serine carboxypeptidase S28 family protein | −2.00 |
| At1g04600 | myosin, putative | −2.00 |
| At2g45790 | eukaryotic phosphomannomutase family protein | −2.01 |
| At1g78200 | protein phosphatase 2C, putative | −2.01 |
| At3g24090 | glucosamine--fructose-6-phosphate aminotransferase (isomerizing), | −2.01 |
| | putative/hexosephosphate aminotransferase, putative/glucosamine-6- | |
| | phosphate synthase, putative/D-fructose-6-phosphate | |
| | amidotransferase, putative/GLCN6P synthase, putative | |
| At1g31580 | expressed protein | −2.01 |
| At3g01440 | oxygen evolving enhancer 3 (PsbQ) family protein | −2.01 |
| At4g35240 | expressed protein | −2.01 |
| At5g10250 | phototropic-responsive protein, putative | −2.01 |
| At1g53870 | expressed protein /// expressed protein | −2.01 |
| At1g80180 | expressed protein | −2.01 |
| At1g27200 | expressed protein | −2.01 |
| At1g06870 | signal peptidase, putative /// signal peptidase, putative | −2.01 |
| At5g06320 | harpin-induced family protein/HIN1 family protein/harpin-responsive | −2.01 |
| | family protein/NDR1/HIN1-like protein 3 | |
| At1g04550 | auxin-responsive protein/indoleacetic acid-induced protein 12 (IAA12) | −2.01 |
| At5g06560 | expressed protein | −2.02 |
| At3g50810 | integral membrane protein, putative | −2.02 |
| At1g50740 | expressed protein | −2.02 |
| At5g53870 | plastocyanin-like domain-containing protein | −2.02 |
| At3g01080 | WRKY family transcription factor | −2.02 |
| At5g49570 | transglutaminase-like family protein | −2.02 |
| At1g32060 | phosphoribulokinase (PRK)/phosphopentokinase | −2.02 |
| At4g11840 | phospholipase D gamma 3/PLD gamma 3 (PLDGAMMA3) | −2.02 |
| At2g41120 | expressed protein | −2.02 |
| At5g47070 | protein kinase, putative | −2.02 |
| At4g23180 | receptor-like protein kinase 4, putative (RLK4) | −2.03 |
| At3g59090 | expressed protein | −2.03 |
| At4g09570 | calcium-dependent protein kinase, putative CDPK | −2.03 |
| At4g26690 | glycerophosphoryl diester phosphodiesterase family protein | −2.03 |
| At1g01790 | K+ efflux antiporter, putative (KEA1) | −2.03 |
| At4g26860 | alanine racemase family protein | −2.03 |
| At1g69530 | expansin, putative (EXP1) | −2.03 |
| At5g07370 | inositol polyphosphate 6-/3-15-kinase 2a (IPK2a) | −2.03 |
| At4g35310 | calcium-dependent protein kinase, putative/CDPK, putative | −2.03 |
| At1g28380 | expressed protein | −2.03 |
| At2g37940 | expressed protein | −2.03 |
| At1g64740 | tubulin alpha-1 chain (TUA1) | −2.03 |
| At1g76580 | SPL1-Related3 protein (SPL1R3) | −2.03 |
| At1g19835 | expressed protein | −2.04 |
| At5g55850 | nitrate-responsive NOI protein, putative | −2.04 |
| At3g07580 | expressed protein | −2.04 |
| At5g13200 | GRAM domain-containing protein/ABA-responsive protein-related | −2.04 |
| At3g14620 | cytochrome P450, putative | −2.04 |
| At1g72700 | haloacid dehalogenase-like hydrolase family protein | −2.04 |
| At2g42360 | zinc finger (C3HC4-type RING finger) family protein | −2.04 |
| At3g28200 | peroxidase, putative | −2.04 |
| At1g10740 | expressed protein | −2.04 |
| At2g40600 | appr-1-p processing enzyme family protein | −2.05 |
| At1g11050 | protein kinase family protein | −2.05 |
| At4g16860 | disease resistance protein (TIR-NBS-LRR class), putative | −2.05 |
| At5g49360 | glycosyl hydrolase family 3 protein | −2.05 |
| At1g53430 | leucine-rich repeat family protein/protein kinase family protein | −2.05 |
| At5g39080 | transferase family protein | −2.05 |
| At3g15760 | expressed protein | −2.05 |
| At2g35760 | integral membrane family protein | −2.06 |
| At5g11090 | serine-rich protein-related | −2.06 |
| At3g05490 | rapid alkalinization factor (RALF) family protein | −2.06 |
| At5g14940 | proton-dependent oligopeptide transport (POT) family protein | −2.06 |
| At1g32870 | no apical meristem (NAM) family protein | −2.06 |
| At3g17410 | serine/threonine protein kinase, putative | −2.06 |
| At1g17340 | phosphoinositide phosphatase family protein | −2.06 |
| At3g26440 | expressed protein | −2.06 |
| At3g13330 | expressed protein | −2.06 |
| At4g17080 | MORN (Membrane Occupation and Recognition Nexus) repeat- | −2.06 |
| | containing protein/phosphatidylinositol-4-phosphate 5-kinase-related | |
| At1g17360 | COP1-interacting protein-related | −2.06 |
| At4g21390 | S-locus lectin protein kinase family protein | −2.07 |
| At4g01330 | protein kinase family protein | −2.07 |
| At1g61140 | SNF2 domain-containing protein/helicase domain-containing protein/ | −2.07 |
| | zinc finger protein-related | |
| At5g57760 | expressed protein | −2.07 |
| At3g08970 | DNAJ heat shock N-terminal domain-containing protein | −2.07 |
| At4g08500 | mitogen-activated protein kinase kinase, putative | −2.07 |
| At5g16570 | glutamine synthetase, putative | −2.07 |
| At5g66050 | expressed protein | −2.07 |
| At4g00490 | beta-amylase, putative/1,4-alpha-D-glucan maltohydrolase, putative | −2.07 |
| At3g04220 | disease resistance protein (TIR-NBS-LRR class), putative | −2.07 |
| At1g32940 | subtilase family protein | −2.08 |
| At1g09970 | leucine-rich repeat transmembrane protein kinase, putative | −2.08 |
| At5g16880 | VHS domain-containing protein/GAT domain-containing protein | −2.08 |
| At2g40750 | WRKY family transcription factor | −2.08 |
| At3g07650 | zinc finger (B-box type) family protein | −2.08 |
| At1g19450 | integral membrane protein, putative/sugar transporter family protein | −2.09 |
| At5g20110 | dynein light chain, putative | −2.09 |
| At1g16880 | uridylyltransferase-related | −2.09 |
| At5g04920 | vacuolar protein sorting 36 family protein/VPS36 family protein | −2.09 |
| At2g30520 | signal transducer of phototropic response (RPT2) | −2.09 |
| At5g18650 | zinc finger (C3HC4-type RING finger) family protein | −2.09 |
| At4g35110 | expressed protein | −2.09 |
| At3g53950 | glyoxal oxidase-related | −2.09 |
| At3g48990 | AMP-dependent synthetase and ligase family protein | −2.09 |
| At4g39640 | gamma-glutamyltranspeptidase family protein | −2.09 |
| At3g50830 | stress-responsive protein, putative | −2.09 |
| At5g46240 | inward rectifying potassium channel (KAT1) | −2.09 |
| At1g14370 | protein kinase (APK2a) | −2.09 |
| At1g76530 | auxin efflux carrier family protein | −2.10 |
| At5g63080 | transcription factor jumonji (jmjC) domain-containing protein | −2.10 |
| At5g61600 | ethylene-responsive element-binding family protein | −2.10 |
| At2g37950 | zinc finger (C3HC4-type RING finger) family protein | −2.10 |
| At4g23130 | receptor-like protein kinase 6 (RLK6) | −2.10 |
| At5g42380 | calmodulin-related protein, putative | −2.10 |
| At2g17500 | auxin efflux carrier family protein | −2.11 |
| At3g08870 | lectin protein kinase, putative | −2.11 |
| At1g65190 | protein kinase family protein | −2.11 |
| At1g49470 | expressed protein | −2.11 |
| At2g30070 | potassium transporter (KUP1) | −2.11 |
| At5g52050 | MATE efflux protein-related | −2.11 |
| At1g01360 | expressed protein | −2.11 |
| At1g18400 | basic helix-loop-helix (bHLH) family protein | −2.11 |
| At3g10500 | no apical meristem (NAM) family protein | −2.11 |
| At5g17300 | myb family transcription factor | −2.11 |
| At3g62410 | CP12 domain-containing protein | −2.11 |
| At5g40670 | PQ-loop repeat family protein/transmembrane family protein | −2.11 |
| At5g62900 | expressed protein | −2.11 |
| At1g58180 | carbonic anhydrase family protein | −2.11 |
| At2g16380 | SEC14 cytosolic factor family protein/phosphoglyceride transfer family | −2.12 |
| | protein | |
| At1g70000 | DNA-binding family protein | −2.12 |
| At5g25140 | cytochrome P450 family protein | −2.12 |
| At3g17770 | dihydroxyacetone kinase family protein | −2.12 |
| At5g19875 | expressed protein | −2.12 |
| At5g03350 | legume lectin family protein | −2.12 |
| At2g03710 | MADS-box protein (AGL3) | −2.12 |
| At5g26200 | mitochondrial substrate carrier family protein | −2.12 |
| At1g69295 | beta-1,3-glucanase-related | −2.12 |
| At2g25625 | expressed protein | −2.13 |
| At3g60320 | expressed protein | −2.13 |
| At2g02420 | expressed protein | −2.13 |
| At4g24570 | mitochondrial substrate carrier family protein | −2.13 |
| At3g27960 | kinesin light chain-related | −2.13 |
| At3g46110 | expressed protein | −2.13 |
| At2g16750 | protein kinase family protein | −2.13 |
| At3g47510 | expressed protein | −2.13 |
| At3g11420 | fringe-related protein | −2.13 |
| At4g39140 | expressed protein | −2.13 |
| At2g30010 | expressed protein | −2.13 |
| At2g23130 | arabinogalactan-protein (AGP17) | −2.14 |
| At5g53450 | protein kinase family protein | −2.14 |
| At1g64610 | WD-40 repeat family protein | −2.14 |
| At5g25280 | serine-rich protein-related | −2.14 |
| At4g24700 | expressed protein | −2.14 |
| At5g62860 | F-box family protein-related | −2.14 |
| At1g44130 | nucellin protein, putative | −2.14 |
| At1g27290 | expressed protein | −2.14 |
| At2g41090 | calmodulin-like calcium-binding protein, 22 kDa (CaBP-22) | −2.14 |
| At1g02170 | latex-abundant family protein (AMC1)/caspase family protein | −2.15 |
| At3g60260 | phagocytosis and cell motility protein ELMO1-related | −2.15 |
| At1g55850 | cellulose synthase family protein | −2.15 |
| At3g51890 | expressed protein | −2.15 |
| At5g55560 | protein kinase family protein | −2.15 |
| At1g51800 | leucine-rich repeat protein kinase, putative | −2.15 |
| At3g54100 | expressed protein | −2.15 |
| At5g60280 | lectin protein kinase family protein | −2.15 |
| At2g44500 | expressed protein | −2.16 |
| At5g35570 | expressed protein | −2.16 |
| At5g13190 | expressed protein | −2.16 |
| At1g33050 | expressed protein | −2.16 |
| At4g27300 | S-locus protein kinase, putative | −2.16 |
| At5g36930 | disease resistance protein (TIR-NBS-LRR class), putative | −2.16 |
| At1g03370 | C2 domain-containing protein/GRAM domain-containing protein | −2.16 |
| At1g03740 | protein kinase family protein | −2.16 |
| At2g42580 | tetratricopeptide repeat (TPR)-containing protein | −2.16 |
| At2g32860 | glycosyl hydrolase family 1 protein | −2.17 |
| At1g67970 | heat shock factor protein, putative (HSF5)/heat shock transcription | −2.17 |
| | factor, putative (HSTF5) | |
| At1g22180 | SEC14 cytosolic factor family protein/phosphoglyceride transfer family | −2.17 |
| | protein | |
| At1g62840 | expressed protein | −2.17 |
| At1g35210 | expressed protein | −2.17 |
| At1g17050 | geranyl diphosphate synthase, putative/GPPS, putative/ | −2.17 |
| | dimethylallyltransferase, putative/prenyl transferase, putative | |
| At4g14220 | zinc finger (C3HC4-type RING finger) family protein | −2.17 |
| At2g03730 | ACT domain-containing protein (ACR5) | −2.17 |
| At1g77810 | galactosyltransferase family protein | −2.17 |
| At5g24810 | ABC1 family protein | −2.17 |
| At3g21080 | ABC transporter-related | −2.17 |
| At5g21170 | 5′-AMP-activated protein kinase beta-2 subunit, putative | −2.17 |
| At4g08930 | thioredoxin-related | −2.17 |
| At1g56120 | leucine-rich repeat family protein/protein kinase family protein | −2.17 |
| At4g33950 | protein kinase, putative | −2.17 |
| At5g26770 | expressed protein | −2.18 |
| At5g13180 | no apical meristem (NAM) family protein | −2.18 |
| At3g07010 | pectate lyase family protein | −2.18 |
| At1g75310 | DNAJ heat shock N-terminal domain-containing protein | −2.18 |
| At1g10470 | two-component responsive regulator/response regulator 4 (ARR4) | −2.18 |
| At3g02170 | expressed protein | −2.18 |
| At5g15830 | bZIP transcription factor family protein | −2.18 |
| At4g23320 | protein kinase family protein | −2.18 |
| At5g45110 | ankyrin repeat family protein/BTB/POZ domain-containing protein | −2.18 |
| At1g32530 | zinc finger (C3HC4-type RING finger) family protein | −2.18 |
| At1g64330 | myosin heavy chain-related | −2.18 |
| At2g01850 | xyloglucan:xyloglucosyl transferase/xyloglucan endotransglycosylase/ | −2.18 |
| | endo-xyloglucan transferase (EXGT-A3) | |
| At4g17250 | expressed protein | −2.19 |
| At5g23510 | expressed protein /// expressed protein | −2.19 |
| At3g47090 | leucine-rich repeat transmembrane protein kinase, putative | −2.19 |
| At4g22980 | expressed protein | −2.19 |
| At1g33110 | MATE efflux family protein | −2.19 |
| At3g16830 | WD-40 repeat family protein | −2.19 |
| At4g12830 | hydrolase, alpha/beta fold family protein | −2.19 |
| At1g59870 | ABC transporter family protein | −2.19 |
| At1g15750 | WD-40 repeat family protein | −2.19 |
| At1g16500 | expressed protein | −2.20 |
| At1g75080 | brassinosteroid signalling positive regulator, putative /// brassinosteroid | −2.20 |
| | signalling positive regulator, putative | |
| At5g46500 | expressed protein | −2.20 |
| At1g65390 | disease resistance protein (TIR class), putative | −2.20 |
| At4g35090 | catalase 2 | −2.20 |
| At3g46900 | copper transporter, putative | −2.20 |
| At1g64360 | expressed protein | −2.21 |
| At3g15310 | expressed protein | −2.21 |
| At5g03520 | Ras-related GTP-binding protein, putative | −2.21 |
| At3g10930 | expressed protein | −2.21 |
| At1g63720 | expressed protein | −2.21 |
| At1g67920 | expressed protein | −2.21 |
| At4g36990 | heat shock transcription factor 4 (HSTF4) | −2.21 |
| At3g53670 | expressed protein | −2.21 |
| At1g18620 | expressed protein | −2.21 |
| At2g17220 | protein kinase, putative | −2.21 |
| At4g37610 | TAZ zinc finger family protein/BTB/POZ domain-containing protein | −2.21 |
| At2g36430 | expressed protein | −2.21 |
| At4g23270 | protein kinase family protein | −2.21 |
| At5g62570 | calmodulin-binding protein | −2.21 |
| At1g11330 | S-locus lectin protein kinase family protein | −2.22 |
| At2g01190 | octicosapeptide/Phox/Bem1p (PB1) domain-containing protein | −2.22 |
| At2g39400 | hydrolase, alpha/beta fold family protein | −2.22 |
| At4g18010 | inositol polyphosphate 5-phosphatase II (IP5PII) | −2.22 |
| At3g17610 | bZIP transcription factor family protein/HY5-like protein (HYH) | −2.22 |
| At1g76990 | ACT domain containing protein | −2.22 |
| At5g66900 | disease resistance protein (CC-NBS-LRR class), putative ve | −2.22 |
| At5g05250 | expressed protein | −2.22 |
| At3g13380 | leucine-rich repeat family protein/protein kinase family protein | −2.22 |
| At1g79700 | ovule development protein, putative | −2.22 |
| At4g00300 | fringe-related protein | −2.22 |
| At5g58670 | phosphoinositide-specific phospholipase C (PLC1) | −2.22 |
| At1g72790 | hydroxyproline-rich glycoprotein family protein | −2.23 |
| At1g33600 | leucine-rich repeat family protein | −2.23 |
| At4g08480 | mitogen-activated protein kinase, putative | −2.23 |
| At2g45170 | autophagy 8e (APG8e) | −2.23 |
| At1g74170 | leucine-rich repeat family protein | −2.23 |
| At3g57230 | MADS-box protein (AGL16) | −2.23 |
| At3g19010 | oxidoreductase, 2OG-Fe(II) oxygenase family protein | −2.23 |
| At2g15830 | expressed protein | −2.23 |
| At3g07610 | transcription factor jumonji (jmjC) domain-containing protein | −2.23 |
| At5g56140 | KH domain-containing protein | −2.23 |
| At5g44070 | phytochelatin synthase 1 (PCS1) | −2.23 |
| At2g31070 | TCP family transcription factor, putative | −2.23 |
| At2g26600 | glycosyl hydrolase family 17 protein | −2.23 |
| At2g34770 | fatty acid hydroxylase (FAH1) | −2.24 |
| At1g05575 | expressed protein | −2.24 |
| At5g22570 | WRKY family transcription factor | −2.24 |
| At3g49790 | expressed protein | −2.24 |
| At5g18240 | myb family transcription factor | −2.24 |
| At1g34760 | 14-3-3 protein GF14 omicron (GRF11) | −2.24 |
| At2g40110 | yippee family protein | −2.24 |
| At1g16670 | protein kinase family protein | −2.24 |
| At2g38210 | ethylene-responsive protein, putative | −2.24 |
| At1g21120 | O-methyltransferase, putative | −2.24 |
| At5g66890 | disease resistance protein (CC-NBS-LRR class), putative | −2.25 |
| At2g22300 | ethylene-responsive calmodulin-binding protein, putative (SR1) | −2.25 |
| At3g18290 | zinc finger protein-related | −2.25 |
| At4g19960 | potassium transporter family protein | −2.25 |
| At3g01350 | proton-dependent oligopeptide transport (POT) family protein | −2.25 |
| At3g55470 | C2 domain-containing protein | −2.26 |
| At2g29460 | glutathione S-transferase, putative | −2.26 |
| At1g29720 | protein kinase family protein | −2.26 |
| At5g61560 | protein kinase family protein | −2.26 |
| At5g67470 | formin homology 2 domain-containing protein/FH2 domain-containing | −2.27 |
| | protein | |
| At5g56750 | Ndr family protein | −2.27 |
| At3g15260 | protein phosphatase 2C, putative/PP2C, putative | −2.27 |
| At4g21400 | protein kinase family protein | −2.27 |
| At5g46490 | disease resistance protein (TIR-NBS-LRR class), putative | −2.27 |
| At1g33240 | trihelix DNA-binding protein, putative | −2.27 |
| At4g12390 | invertase/pectin methylesterase inhibitor family protein | −2.27 |
| At3g04640 | glycine-rich protein | −2.28 |
| At2g30550 | lipase class 3 family protein | −2.28 |
| At5g46410 | NLI interacting factor (NIF) family protein | −2.28 |
| At5g57560 | xyloglucan:xyloglucosyl transferase/xyloglucan endotransglycosylase/ | −2.28 |
| | endo-xyloglucan transferase (TCH4) | |
| psbB | — | −2.28 |
| At2g46330 | arabinogalactan-protein (AGP16) | −2.28 |
| At2g43430 | hydroxyacylglutathione hydrolase, mitochondrial/glyoxalase II (GLX2-1) | −2.28 |
| At2g04070 | MATE efflux family protein | −2.28 |
| At5g43190 | F-box family protein (FBX6) | −2.29 |
| At5g44290 | protein kinase family protein | −2.29 |
| At2g41730 | expressed protein | −2.29 |
| At1g76700 | DNAJ heat shock N-terminal domain-containing protein | −2.29 |
| At2g43820 | UDP-glucoronosyl/UDP-glucosyl transferase family protein | −2.29 |
| At4g16000 | expressed protein | −2.29 |
| At3g54960 | thioredoxin family protein | −2.29 |
| At1g71697 | choline kinase, putative | −2.30 |
| At5g54380 | protein kinase family protein | −2.30 |
| At2g29720 | monooxygenase family protein | −2.30 |
| At5g09290 | 3′(2′),5′-bisphosphate nucleotidase, putative/inositol polyphosphate 1- | −2.31 |
| | phosphatase, putative | |
| At2g40670 | two-component responsive regulator/response regulator 16 (ARR16) | −2.31 |
| At3g57480 | zinc finger (C2H2 type, AN1-like) family protein | −2.31 |
| At5g66200 | armadillo/beta-catenin repeat family protein | −2.31 |
| At4g29810 | mitogen-activated protein kinase kinase (MAPKK) (MKK2) | −2.31 |
| At5g56860 | zinc finger (GATA type) family protein | −2.31 |
| At1g32700 | zinc-binding family protein | −2.31 |
| At3g47010 | glycosyl hydrolase family 3 protein | −2.31 |
| At1g59620 | disease resistance protein (CC-NBS class), putative | −2.32 |
| At1g48300 | expressed protein | −2.32 |
| At3g59080 | aspartyl protease family protein | −2.32 |
| At5g65730 | xyloglucan:xyloglucosyl transferase, putative/xyloglucan | −2.32 |
| | endotransglycosylase, putative/endo-xyloglucan transferase, putative | |
| At1g75410 | BEL1-like homeodomain 3 protein (BLH3) | −2.32 |
| At1g75170 | SEC14 cytosolic factor family protein/phosphoglyceride transfer family | −2.32 |
| | protein | |
| At1g42990 | bZIP transcription factor family protein | −2.32 |
| At5g10650 | zinc finger (C3HC4-type RING finger) family protein | −2.32 |
| At4g22710 | cytochrome P450 family protein | −2.33 |
| At1g21270 | wall-associated kinase 2 (WAK2) | −2.33 |
| At4g18880 | heat shock transcription factor 21 (HSF21) | −2.33 |
| At2g21185 | expressed protein | −2.33 |
| At2g01860 | pentatricopeptide (PPR) repeat-containing protein | −2.33 |
| At1g62420 | expressed protein | −2.34 |
| At1g05570 | callose synthase 1 (CALS1)/1,3-beta-glucan synthase 1 | −2.34 |
| At3g45620 | transducin family protein/WD-40 repeat family protein | −2.34 |
| At3g16857 | two-component responsive regulator family protein/response regulator | −2.35 |
| | family protein | |
| At3g05900 | neurofilament protein-related | −2.35 |
| At1g53570 | mitogen-activated protein kinase kinase kinase (MAPKKK), putative | −2.35 |
| | (MAP3Ka) | |
| At1g23870 | glycosyl transferase family 20 protein/trehalose-phosphatase family | −2.35 |
| | protein | |
| At3g51920 | calmodulin-9 (CAM9) | −2.35 |
| At1g80130 | expressed protein | −2.35 |
| At3g04030 | myb family transcription factor | −2.35 |
| At1g51270 | vesicle-associated membrane protein, putative/VAMP | −2.35 |
| At1g26960 | homeobox-leucine zipper protein, putative/HD-ZIP transcription factor, | −2.35 |
| | putative | |
| At1g03210 | phenazine biosynthesis PhzC/PhzF family protein | −2.35 |
| At5g05460 | glycosyl hydrolase family 85 protein | −2.36 |
| At1g77760 | nitrate reductase 1 (NR1) | −2.36 |
| At1g31350 | F-box family protein | −2.36 |
| At1g61180 | disease resistance protein (CC-NBS-LRR class), putative | −2.36 |
| At4g28085 | expressed protein | −2.36 |
| At2g15040 | pseudogene, disease resistance protein-related | −2.36 |
| At1g74330 | protein kinase, putative | −2.37 |
| At5g62620 | galactosyltransferase family protein | −2.37 |
| At1g23830 | expressed protein | −2.37 |
| At5g08240 | expressed protein | −2.37 |
| At2g18230 | inorganic pyrophosphatase (soluble) (PPA)/pyrophosphate phospho- | −2.38 |
| | hydrolase/PPase | |
| At2g12290 | hypothetical protein /// hypothetical protein | −2.38 |
| At1g22510 | zinc finger (C3HC4-type RING finger) family protein | −2.38 |
| At5g67420 | LOB domain protein 37/lateral organ boundaries domain protein 37 | −2.38 |
| | (LBD37) | |
| At4g17350 | expressed protein | −2.38 |
| At4g23480 | hydroxyproline-rich glycoprotein family protein | −2.38 |
| At1g44100 | amino acid permease 5, putative (AAP5) | −2.38 |
| At5g65210 | bZIP family transcription factor (TGA1) | −2.38 |
| At1g77900 | expressed protein | −2.38 |
| At3g57530 | calcium-dependent protein kinase, putative/CDPK, putative | −2.38 |
| At1g75020 | phospholipid/glycerol acyltransferase family protein | −2.39 |
| At1g08570 | thioredoxin family protein | −2.39 |
| At5g27350 | sugar-porter family protein 1 (SFP1) | −2.40 |
| At1g78510 | solanesyl diphosphate synthase (SPS) | −2.40 |
| At4g33010 | glycine dehydrogenase (decarboxylating), putative/glycine | −2.40 |
| | decarboxylase, putative/glycine cleavage system P-protein, putative | |
| At1g63890 | zinc finger (C3HC4-type RING finger) family protein | −2.40 |
| At5g47220 | ethylene-responsive element-binding factor 2 (ERF2) | −2.40 |
| At3g28180 | glycosyl transferase family 2 protein | −2.40 |
| At1g67810 | Fe—S metabolism associated domain-containing protein | −2.41 |
| At5g46270 | disease resistance protein (TIR-NBS-LRR class), putative | −2.41 |
| At1g15890 | disease resistance protein (CC-NBS-LRR class), putative | −2.41 |
| At4g26060 | expressed protein | −2.42 |
| At1g30260 | expressed protein | −2.42 |
| At3g63420 | heterotrimeric G protein gamma-subunit (AGG1) | −2.42 |
| At4g38260 | expressed protein | −2.42 |
| At3g47250 | expressed protein | −2.42 |
| At2g24550 | expressed protein | −2.43 |
| At2g44240 | expressed protein | −2.43 |
| At4g10960 | lipase class 3 family protein | −2.43 |
| At1g78860 | curculin-like (mannose-binding) lectin family protein | −2.43 |
| At1g67850 | expressed protein | −2.43 |
| At4g26090 | disease resistance protein RPS2 (CC-NBS-LRR class), putative | −2.44 |
| At5g40890 | chloride channel protein (CLC-a) | −2.44 |
| At5g45500 | expressed protein | −2.44 |
| At5g50200 | expressed protein | −2.44 |
| At4g36520 | trichohyalin-related | −2.44 |
| At4g36500 | expressed protein | −2.44 |
| At1g74440 | expressed protein | −2.44 |
| At3g22910 | calcium-transporting ATPase, plasma membrane-type, putative (ACA13) | −2.45 |
| At5g54490 | calcium-binding EF-hand protein, putative | −2.45 |
| At1g12580 | protein kinase family protein | −2.45 |
| At3g52800 | zinc finger (AN1-like) family protein | −2.45 |
| At4g25000 | alpha-amylase, putative/1,4-alpha-D-glucan glucanohydrolase, putative | −2.45 |
| At5g37600 | glutamine synthetase, putative | −2.46 |
| At4g34480 | glycosyl hydrolase family 17 protein | −2.46 |
| At4g33920 | protein phosphatase 2C family protein/PP2C family protein | −2.46 |
| At4g35780 | protein kinase family protein | −2.46 |
| At1g04040 | acid phosphatase class B family protein | −2.46 |
| At2g45910 | protein kinase family protein/U-box domain-containing protein | −2.47 |
| At4g30850 | expressed protein | −2.47 |
| At2g01180 | phosphatidic acid phosphatase family protein/PAP2 family protein | −2.47 |
| At3g06080 | expressed protein | −2.47 |
| At1g77990 | sulfate transporter | −2.47 |
| At1g49050 | aspartyl protease family protein | −2.47 |
| At1g29240 | expressed protein | −2.48 |
| At3g48850 | mitochondrial phosphate transporter, putative | −2.48 |
| At3g06150 | expressed protein | −2.48 |
| At5g65010 | asparagine synthetase 2 (ASN2) | −2.48 |
| At3g63380 | calcium-transporting ATPase, plasma membrane-type, putative/Ca(2+)- | −2.49 |
| | ATPase, putative (ACA12) | |
| At3g07780 | expressed protein | −2.49 |
| At5g59680 | leucine-rich repeat protein kinase, putative | −2.50 |
| At5g56870 | beta-galactosidase, putative/lactase, putative | −2.50 |
| At1g29660 | GDSL-motif lipase/hydrolase family protein | −2.51 |
| At4g16690 | esterase/lipase/thioesterase family protein | −2.51 |
| At1g66970 | glycerophosphoryl diester phosphodiesterase family protein | −2.51 |
| At1g71880 | sucrose transporter/sucrose-proton symporter (SUC1) | −2.51 |
| At1g59590 | expressed protein | −2.51 |
| At5g45550 | mob1/phocein family protein | −2.51 |
| At2g26080 | glycine dehydrogenase (decarboxylating), putative/glycine | −2.52 |
| | decarboxylase, putative/glycine cleavage system P-protein, putative | |
| At5g51630 | disease resistance protein (TIR-NBS-LRR class), putative | −2.52 |
| At2g41110 | calmodulin-2/3/5 (CAM2) (CAL1) | −2.52 |
| At5g02490 | heat shock cognate 70 kDa protein 2 (HSC70-2) (HSP70-2) | −2.52 |
| At1g21130 | O-methyltransferase, putative | −2.52 |
| At3g57700 | protein kinase, putative | −2.52 |
| At5g59450 | scarecrow-like transcription factor 11 (SCL11) | −2.52 |
| At3g56710 | sigA-binding protein | −2.53 |
| At1g71330 | ABC transporter family protein | −2.53 |
| At5g53970 | aminotransferase, putative | −2.53 |
| At4g18740 | expressed protein | −2.53 |
| At1g17620 | expressed protein | −2.53 |
| At3g28580 | AAA-type ATPase family protein | −2.53 |
| At4g32870 | expressed protein | −2.53 |
| At4g37790 | homeobox-leucine zipper protein 22 (HAT22)/HD-ZIP protein 22 | −2.53 |
| At5g56250 | expressed protein | −2.54 |
| At1g26270 | phosphatidylinositol 3- and 4-kinase family protein | −2.54 |
| At3g56200 | amino acid transporter family protein | −2.54 |
| At2g38790 | expressed protein | −2.55 |
| At4g00150 | scarecrow-like transcription factor 6 (SCL6) | −2.55 |
| At1g55910 | metal transporter, putative (ZIP11) | −2.55 |
| At4g27280 | calcium-binding EF hand family protein | −2.55 |
| At5g44820 | expressed protein | −2.55 |
| At1g72940 | disease resistance protein (TIR-NBS class), putative | −2.55 |
| At4g37640 | calcium-transporting ATPase 2, plasma membrane-type/Ca(2+)- | −2.55 |
| | ATPase isoform 2 (ACA2) | |
| At2g17550 | expressed protein | −2.56 |
| At2g37460 | nodulin MtN21 family protein | −2.56 |
| At1g25550 | myb family transcription factor | −2.56 |
| At1g63880 | disease resistance protein (TIR-NBS-LRR class), putative | −2.56 |
| At2g27830 | expressed protein | −2.57 |
| At5g10695 | expressed protein | −2.57 |
| At2g23320 | WRKY family transcription factor | −2.57 |
| At4g23260 | protein kinase family protein | −2.57 |
| At2g06850 | xyloglucan:xyloglucosyl transferase/xyloglucan endotransglycosylase/ | −2.57 |
| | endo-xyloglucan transferase (EXT) (EXGT-A1) | |
| At1g09560 | germin-like protein (GLP4) (GLP5) | −2.57 |
| At2g40270 | protein kinase family protein | −2.57 |
| At3g60160 | ABC transporter family protein /// ABC transporter family protein | −2.57 |
| At5g61590 | AP2 domain-containing transcription factor family protein | −2.58 |
| At2g17440 | leucine-rich repeat family protein | −2.58 |
| At3g28510 | AAA-type ATPase family protein | −2.58 |
| At5g40850 | urophorphyrin III methylase (UPM1) | −2.58 |
| At4g20070 | peptidase M20/M25/M40 family protein | −2.58 |
| At1g04200 | expressed protein | −2.59 |
| At2g24850 | aminotransferase, putative | −2.59 |
| At1g05840 | aspartyl protease family protein | −2.59 |
| At4g20780 | calcium-binding protein, putative | −2.59 |
| At1g17610 | disease resistance protein-related | −2.59 |
| At1g69840 | band 7 family protein | −2.60 |
| At3g11280 | myb family transcription factor | −2.60 |
| At3g63080 | glutathione peroxidase, putative | −2.61 |
| At1g03400 | 2-oxoglutarate-dependent dioxygenase, putative | −2.61 |
| At1g18210 | calcium-binding protein, putative | −2.61 |
| At4g17900 | zinc-binding family protein | −2.61 |
| At4g16370 | oligopeptide transporter OPT family protein | −2.62 |
| At2g28400 | expressed protein | −2.62 |
| At5g56340 | zinc finger (C3HC4-type RING finger) family protein | −2.62 |
| At2g19710 | expressed protein | −2.62 |
| At3g24503 | aldehyde dehydrogenase (ALDH1a) | −2.62 |
| At3g45260 | zinc finger (C2H2 type) family protein | −2.62 |
| At3g16220 | expressed protein | −2.63 |
| At5g51460 | trehalose-6-phosphate phosphatase (TPPA) | −2.63 |
| At2g43850 | ankyrin protein kinase, putative (APK1) | −2.63 |
| At4g29050 | lectin protein kinase family protein | −2.63 |
| At1g60140 | glycosyl transferase family 20 protein/trehalose-phosphatase family | −2.63 |
| | protein | |
| At5g05190 | expressed protein | −2.63 |
| At1g79270 | expressed protein | −2.63 |
| At2g40100 | chlorophyll A-B binding protein (LHCB4.3) | −2.63 |
| At1g61100 | disease resistance protein (TIR class), putative | −2.64 |
| At5g44870 | disease resistance protein (TIR-NBS-LRR class), putative | −2.64 |
| At1g24530 | transducin family protein/WD-40 repeat family protein | −2.64 |
| At1g05000 | tyrosine specific protein phosphatase family protein | −2.64 |
| At4g23210 | protein kinase family protein | −2.64 |
| At1g23030 | armadillo/beta-catenin repeat family protein/U-box domain-containing | −2.64 |
| | protein | |
| At4g08950 | phosphate-responsive protein, putative (EXO) | −2.65 |
| At1g71040 | multi-copper oxidase type I family protein | −2.65 |
| At3g55950 | protein kinase family protein | −2.65 |
| At4g02330 | pectinesterase family protein | −2.66 |
| At3g45290 | seven transmembrane MLO family protein/MLO-like protein 3 (MLO3) | −2.66 |
| At1g03020 | glutaredoxin family protein | −2.66 |
| At3g56400 | WRKY family transcription factor | −2.66 |
| At1g51660 | mitogen-activated protein kinase kinase (MAPKK), putative (MKK4) | −2.66 |
| At1g03850 | glutaredoxin family protein | −2.67 |
| At3g61190 | BON1-associated protein 1 (BAP1) | −2.67 |
| At4g01720 | WRKY family transcription factor | −2.67 |
| At3g05200 | zinc finger (C3HC4-type RING finger) family protein (ATL6) | −2.67 |
| At2g17040 | no apical meristem (NAM) family protein | −2.67 |
| At5g04720 | disease resistance protein (CC-NBS-LRR class), putative | −2.67 |
| At2g18700 | glycosyl transferase family 20 protein/trehalose-phosphatase family | −2.67 |
| | protein | |
| At4g21940 | calcium-dependent protein kinase, putative/CDPK, putative | −2.68 |
| At2g31110 | expressed protein | −2.68 |
| At5g15730 | serine/threonine protein kinase, putative | −2.68 |
| At1g22930 | T-complex protein 11 | −2.68 |
| At2g33530 | serine carboxypeptidase S10 family protein | −2.68 |
| At4g26070 | mitogen-activated protein kinase kinase (MAPKK) (MKK1) (MEK1) | −2.68 |
| At3g22930 | calmodulin, putative | −2.69 |
| At5g15870 | glycosyl hydrolase family 81 protein | −2.69 |
| At2g18300 | basic helix-loop-helix (bHLH) family protein | −2.70 |
| At1g22280 | protein phosphatase 2C, putative/PP2C, putative | −2.70 |
| At1g15010 | expressed protein | −2.71 |
| At5g44130 | fasciclin-like arabinogalactan-protein, putative | −2.71 |
| At5g42050 | expressed protein | −2.71 |
| At1g18890 | calcium-dependent protein kinase 1 (CDPK1) | −2.71 |
| At2g47730 | glutathione S-transferase 6 (GST6) | −2.71 |
| At1g27730 | zinc finger (C2H2 type) family protein (ZAT10)/salt-tolerance zinc finger | −2.72 |
| | protein (STZ) | |
| At3g15770 | expressed protein | −2.72 |
| At1g79380 | copine-related | −2.72 |
| At2g44080 | expressed protein | −2.72 |
| At2g26980 | CBL-interacting protein kinase 3 (CIPK3) | −2.72 |
| At3g13062 | expressed protein | −2.72 |
| At4g29950 | microtubule-associated protein | −2.72 |
| At5g05960 | protease inhibitor/seed storage/lipid transfer protein (LTP) family protein | −2.72 |
| At3g49780 | phytosulfokines 3 (PSK3) | −2.73 |
| At2g15320 | leucine-rich repeat family protein | −2.73 |
| At3g07980 | protein kinase, putative | −2.73 |
| At4g23190 | protein kinase family protein | −2.73 |
| At5g11000 | expressed protein | −2.74 |
| At5g14120 | nodulin family protein | −2.74 |
| At5g37740 | C2 domain-containing protein | −2.74 |
| At3g22231 | expressed protein | −2.76 |
| At5g20830 | sucrose synthase/sucrose-UDP glucosyltransferase (SUS1) | −2.76 |
| At1g16510 | auxin-responsive family protein | −2.76 |
| At1g01140 | CBL-interacting protein kinase 9 (CIPK9) | −2.76 |
| At1g34420 | leucine-rich repeat family protein/protein kinase family protein | −2.76 |
| At5g64410 | oligopeptide transporter OPT family protein | −2.76 |
| At1g05630 | endonuclease/exonuclease/phosphatase family protein | −2.77 |
| At1g48320 | thioesterase family protein | −2.77 |
| At1g68690 | pseudogene, protein kinase family | −2.77 |
| At2g28200 | zinc finger (C2H2 type) family protein | −2.77 |
| At1g16170 | expressed protein | −2.77 |
| At1g20160 | subtilase family protein | −2.78 |
| At1g51280 | disease resistance protein (TIR class), putative | −2.78 |
| At5g38210 | serine/threonine protein kinase family protein | −2.78 |
| At4g28270 | zinc finger (C3HC4-type RING finger) family protein | −2.79 |
| At4g17070 | expressed protein | −2.79 |
| At1g23140 | C2 domain-containing protein | −2.80 |
| At3g50950 | disease resistance protein (CC-NBS-LRR class), putative | −2.80 |
| At3g57550 | guanylate kinase 2 (GK-2) | −2.80 |
| At4g30110 | ATPase E1-E2 type family protein/haloacid dehalogenase-like | −2.80 |
| | hydrolase family protein/heavy-metal-associated domain-containing | |
| | protein | |
| At3g29240 | expressed protein | −2.81 |
| At1g22530 | SEC14 cytosolic factor family protein/phosphoglyceride transfer family | −2.81 |
| | protein | |
| At4g11850 | phospholipase D gamma 1/PLD gamma 1 (PLDGAMMA1) | −2.81 |
| At1g70250 | receptor serine/threonine kinase, putative | −2.81 |
| At2g37540 | short-chain dehydrogenase/reductase (SDR) family protein | −2.81 |
| At3g56980 | basic helix-loop-helix (bHLH) family protein | −2.81 |
| At1g12200 | flavin-containing monooxygenase family protein/FMO family protein | −2.82 |
| At3g05650 | disease resistance family protein | −2.82 |
| At1g21310 | proline-rich extensin-like family protein | −2.82 |
| At1g70940 | auxin transport protein, putative (PIN3) | −2.82 |
| At3g56290 | expressed protein | −2.83 |
| At5g18490 | expressed protein | −2.83 |
| At4g25940 | epsin N-terminal homology (ENTH) domain-containing protein | −2.84 |
| At3g47780 | ABC transporter family protein | −2.84 |
| At1g15790 | expressed protein | −2.84 |
| At5g05440 | expressed protein | −2.84 |
| At1g25560 | AP2 domain-containing transcription factor, putative | −2.84 |
| At3g23880 | F-box family protein | −2.84 |
| At4g33420 | peroxidase, putative | −2.84 |
| At5g23210 | serine carboxypeptidase S10 family protein | −2.84 |
| At1g17990 | 12-oxophytodienoate reductase, putative | −2.85 |
| At1g03290 | expressed protein | −2.85 |
| At2g33580 | protein kinase family protein/peptidoglycan-binding LysM domain- | −2.85 |
| | containing protein | |
| At3g12740 | LEM3 (ligand-effect modulator 3) family protein/CDC50 family protein | −2.86 |
| At4g00970 | protein kinase family protein | −2.87 |
| At5g39050 | transferase family protein | −2.87 |
| At4g17660 | protein kinase, putative | −2.88 |
| At1g69270 | leucine-rich repeat family protein/protein kinase family protein | −2.88 |
| At1g72430 | auxin-responsive protein-related | −2.89 |
| At1g72930 | disease resistance protein (TIR-NBS class), putative | −2.89 |
| At3g09830 | protein kinase, putative | −2.90 |
| At1g23840 | expressed protein | −2.90 |
| At1g22890 | expressed protein | −2.91 |
| At2g48030 | endonuclease/exonuclease/phosphatase family protein | −2.91 |
| At5g04930 | phospholipid-transporting ATPase 1/aminophospholipid flippase 1/ | −2.91 |
| | magnesium-ATPase 1 (ALA1) | |
| At1g52200 | expressed protein | −2.91 |
| At1g77510 | protein disulfide isomerase, putative | −2.91 |
| At3g46130 | myb family transcription factor (MYB48) | −2.92 |
| At3g28007 | nodulin MtN3 family protein | −2.93 |
| At2g37970 | SOUL heme-binding family protein | −2.94 |
| At2g19130 | S-locus lectin protein kinase family protein | −2.94 |
| At3g61210 | embryo-abundant protein-related | −2.94 |
| At2g29990 | pyridine nucleotide-disulphide oxidoreductase family protein | −2.95 |
| At3g21520 | expressed protein | −2.95 |
| At3g26230 | cytochrome P450 family protein | −2.95 |
| At1g66160 | U-box domain-containing protein | −2.95 |
| At4g17090 | beta-amylase (CT-BMY)/1,4-alpha-D-glucan maltohydrolase | −2.96 |
| At4g32300 | lectin protein kinase family protein | −2.96 |
| At1g72300 | leucine-rich repeat transmembrane protein kinase, putative | −2.97 |
| At3g09020 | alpha 1,4-glycosyltransferase family protein/glycosyltransferase sugar- | −2.97 |
| | binding DXD motif-containing protein | |
| At4g21830 | methionine sulfoxide reductase domain-containing protein/SelR | −2.97 |
| | domain-containing protein | |
| At2g23680 | stress-responsive protein, putative | −2.97 |
| At4g33300 | disease resistance protein (CC-NBS-LRR class), putative | −2.98 |
| At3g07720 | kelch repeat-containing protein | −2.98 |
| At1g05340 | expressed protein | −2.98 |
| At2g34510 | expressed protein | −2.98 |
| At1g52290 | protein kinase family protein | −2.99 |
| At5g20250 | raffinose synthase family protein/seed imbibition protein, putative | −3.00 |
| | (din10) | |
| At4g23240 | protein kinase family protein | −3.00 |
| At5g46450 | disease resistance protein (TIR-NBS-LRR class), putative | −3.00 |
| At1g12290 | disease resistance protein (CC-NBS-LRR class), putative | −3.00 |
| At5g60900 | lectin protein kinase family protein | −3.01 |
| At1g21000 | zinc-binding family protein | −3.01 |
| At5g38900 | DSBA oxidoreductase family protein | −3.01 |
| At3g28340 | galactinol synthase, putative | −3.02 |
| At3g25600 | calmodulin, putative | −3.03 |
| At1g72360 | ethylene-responsive element-binding protein, putative | −3.04 |
| At4g35600 | protein kinase family protein | −3.04 |
| At1g26420 | FAD-binding domain-containing protein | −3.04 |
| At3g09010 | protein kinase family protein | −3.04 |
| At5g54860 | integral membrane transporter family protein | −3.05 |
| At3g01550 | triose phosphate/phosphate translocator, putative | −3.05 |
| At4g13510 | ammonium transporter 1, member 1 (AMT1.1) | −3.05 |
| At1g55265 | expressed protein | −3.05 |
| At2g04795 | expressed protein | −3.05 |
| At3g48640 | expressed protein | −3.07 |
| At4g31390 | ABC1 family protein | −3.08 |
| At5g25190 | ethylene-responsive element-binding protein, putative | −3.08 |
| At5g11970 | expressed protein | −3.08 |
| At2g01670 | MutT/nudix family protein | −3.09 |
| At4g00330 | protein kinase family protein | −3.09 |
| At5g45340 | cytochrome P450 family protein | −3.09 |
| At3g26590 | MATE efflux family protein | −3.09 |
| At3g19930 | sugar transport protein (STP4) | −3.10 |
| At1g34750 | protein phosphatase 2C, putative/PP2C, putative | −3.10 |
| At5g46330 | leucine-rich repeat transmembrane protein kinase, putative | −3.11 |
| At2g32030 | GCN5-related N-acetyltransferase (GNAT) family protein | −3.11 |
| At3g55500 | expansin, putative (EXP16) | −3.11 |
| At2g41180 | sigA-binding protein-related | −3.12 |
| At4g15530 | pyruvate phosphate dikinase family protein | −3.13 |
| At4g22530 | embryo-abundant protein-related | −3.13 |
| At1g52870 | peroxisomal membrane protein-related | −3.13 |
| At3g07870 | F-box family protein | −3.14 |
| At3g62150 | multidrug resistant (MDR) ABC transporter, putative | −3.15 |
| At2g29350 | tropinone reductase, putative | −3.16 |
| At1g18390 | protein kinase family protein | −3.16 |
| At1g67910 | expressed protein | −3.17 |
| At4g39070 | zinc finger (B-box type) family protein | −3.17 |
| At5g39020 | protein kinase family protein | −3.17 |
| At1g35140 | phosphate-responsive protein, putative | −3.17 |
| At4g17245 | zinc finger (C3HC4-type RING finger) family protein | −3.18 |
| At2g39360 | protein kinase family protein | −3.18 |
| At1g68840 | DNA-binding protein RAV2 (RAV2)/AP2 domain-containing protein | −3.19 |
| | RAP2.8 | |
| At5g57220 | cytochrome P450, putative | −3.19 |
| At2g17120 | peptidoglycan-binding LysM domain-containing protein | −3.20 |
| At5g45460 | expressed protein /// expressed protein | −3.20 |
| At1g68440 | expressed protein | −3.20 |
| At5g61210 | SNAP25 homologous protein SNAP33 (SNAP33) (SNAP33B)/ | −3.21 |
| | synaptosomal-associated protein SNAP25-like 1/snap25a | |
| At5g42760 | O-methyltransferase N-terminus domain-containing protein | −3.21 |
| At1g78450 | SOUL heme-binding family protein | −3.23 |
| At1g63900 | zinc finger (C3HC4-type RING finger) family protein | −3.23 |
| At1g78820 | curculin-like (mannose-binding) lectin family protein/PAN domain- | −3.23 |
| | containing protein | |
| At5g67370 | expressed protein | −3.23 |
| At1g66960 | lupeol synthase, putative/2,3-oxidosqualene-triterpenoid cyclase, | −3.24 |
| | putative | |
| At5g15850 | zinc finger protein CONSTANS-LIKE 1 (COL1) | −3.24 |
| At5g03120 | expressed protein | −3.24 |
| At5g25350 | F-box family protein | −3.25 |
| At1g17600 | disease resistance protein (TIR-NBS-LRR class), putative | −3.25 |
| At4g13810 | disease resistance family protein/LRR family protein | −3.25 |
| At3g28210 | zinc finger (AN1-like) family protein | −3.28 |
| At3g28540 | AAA-type ATPase family protein | −3.28 |
| At2g30770 | cytochrome P450 71A13, putative (CYP71A13) | −3.28 |
| At4g33790 | acyl CoA reductase, putative | −3.28 |
| At5g62165 | MADS-box protein (AGL42) | −3.30 |
| At1g30410 | ATP-binding cassette transport protein, putative | −3.30 |
| At5g14760 | L-aspartate oxidase family protein | −3.30 |
| At1g76960 | expressed protein | −3.30 |
| At4g11000 | ankyrin repeat family protein | −3.30 |
| At3g11840 | U-box domain-containing protein | −3.31 |
| At1g35350 | EXS family protein/ERD1/XPR1/SYG1 family protein | −3.31 |
| At2g27920 | serine carboxypeptidase S10 family protein | −3.32 |
| At5g02290 | protein kinase, putative | −3.33 |
| At1g80840 | WRKY family transcription factor | −3.34 |
| At5g49730 | ferric reductase-like transmembrane component family protein | −3.36 |
| At1g56150 | auxin-responsive family protein | −3.36 |
| At3g55980 | zinc finger (CCCH-type) family protein | −3.37 |
| At4g01700 | chitinase, putative | −3.37 |
| At3g11820 | syntaxin 121 (SYP121)/syntaxin-related protein (SYR1) | −3.37 |
| At1g59710 | expressed protein | −3.38 |
| At4g11900 | S-locus lectin protein kinase family protein | −3.38 |
| At5g66210 | calcium-dependent protein kinase family protein CDPK | −3.38 |
| At4g08850 | leucine-rich repeat family protein/protein kinase family protein | −3.40 |
| At5g45000 | Toll-Interleukin-Resistance (TIR) domain-containing protein | −3.40 |
| At5g55420 | hypothetical protein | −3.41 |
| At1g69720 | heme oxygenase 3 (HO3) | −3.41 |
| At4g20830 | FAD-binding domain-containing protein | −3.42 |
| At2g22500 | mitochondrial substrate carrier family protein | −3.42 |
| At5g44810 | expressed protein | −3.43 |
| At5g48380 | leucine-rich repeat family protein/protein kinase family protein | −3.43 |
| At3g06070 | expressed protein | −3.44 |
| At1g37130 | nitrate reductase 2 (NR2) | −3.45 |
| At3g47480 | calcium-binding EF hand family protein | −3.45 |
| At4g39800 | inositol-3-phosphate synthase isozyme 1/myo-inositol-1-phosphate | −3.45 |
| | synthase 1/MI-1-P synthase 1/IPS 1 | |
| At3g60290 | oxidoreductase, 2OG-Fe(II) oxygenase family protein | −3.45 |
| At4g16990 | disease resistance protein (TIR-NBS class), putative | −3.46 |
| At3g48720 | transferase family protein | −3.47 |
| At4g05020 | NADH dehydrogenase-related | −3.47 |
| At3g56410 | expressed protein | −3.48 |
| At4g35180 | amino acid transporter family protein | −3.49 |
| At3g44630 | disease resistance protein RPP1-WsB-like (TIR-NBS-LRR class), | −3.49 |
| | putative | |
| At3g07340 | basic helix-loop-helix (bHLH) family protein | −3.49 |
| At4g08470 | mitogen-activated protein kinase, putative | −3.52 |
| At5g40780 | lysine and histidine specific transporter, putative | −3.52 |
| At5g47560 | sodium/dicarboxylate cotransporter, putative | −3.53 |
| At3g30180 | cytochrome P450, putative | −3.54 |
| psbT | — | −3.54 |
| At2g23810 | senescence-associated family protein | −3.54 |
| At3g21870 | cyclin family protein | −3.54 |
| At2g32540 | cellulose synthase family protein | −3.54 |
| At5g45380 | sodium:solute symporter family protein | −3.55 |
| At1g76890 | trihelix DNA-binding protein/GT-2 factor (GT2) | −3.56 |
| At1g54740 | expressed protein | −3.56 |
| At5g27420 | zinc finger (C3HC4-type RING finger) family protein | −3.56 |
| At1g10550 | xyloglucan:xyloglucosyl transferase, putative/xyloglucan | −3.57 |
| | endotransglycosylase, putative/endo-xyloglucan transferase, putative | |
| At5g25440 | protein kinase family protein | −3.57 |
| At3g44400 | disease resistance protein (TIR-NBS-LRR class), putative | −3.57 |
| At1g68570 | proton-dependent oligopeptide transport (POT) family protein | −3.58 |
| psbI | — | −3.58 |
| At2g25735 | expressed protein | −3.60 |
| At5g24110 | WRKY family transcription factor | −3.60 |
| At5g61010 | exocyst subunit EXO70 family protein | −3.61 |
| At4g39030 | enhanced disease susceptibility 5 (EDS5)/salicylic acid induction | −3.63 |
| | deficient 1 (SID1) | |
| At3g16530 | legume lectin family protein | −3.65 |
| At1g55450 | embryo-abundant protein-related | −3.66 |
| At1g30250 | expressed protein | −3.66 |
| At3g53150 | UDP-glucoronosyl/UDP-glucosyl transferase family protein | −3.70 |
| At3g45640 | mitogen-activated protein kinase, putative/MAPK, putative (MPK3) | −3.70 |
| At4g39890 | Ras-related GTP-binding family protein | −3.71 |
| At3g48090 | disease resistance protein (EDS1) | −3.72 |
| At3g60470 | hypothetical protein | −3.72 |
| At2g19650 | DC1 domain-containing protein | −3.74 |
| At1g71390 | disease resistance family protein/LRR family protein | −3.75 |
| At1g24140 | matrixin family protein | −3.76 |
| At1g22740 | Ras-related protein (RAB7)/AtRab75/small GTP-binding protein, | −3.76 |
| | putative | |
| At5g02760 | protein phosphatase 2C family protein/PP2C family protein | −3.78 |
| At5g58770 | dehydrodolichyl diphosphate synthase, putative/DEDOL-PP synthase, | −3.78 |
| | putative | |
| At3g04210 | disease resistance protein (TIR-NBS class), putative | −3.79 |
| At1g67470 | protein kinase family protein | −3.79 |
| At4g00955 | expressed protein | −3.79 |
| At1g33260 | protein kinase family protein | −3.81 |
| At2g35980 | harpin-induced family protein (YLS9)/HIN1 family protein/harpin- | −3.83 |
| | responsive family protein | |
| At4g13340 | leucine-rich repeat family protein/extensin family protein | −3.84 |
| At4g38550 | expressed protein | −3.85 |
| At5g41790 | COP1-interactive protein 1/CIP1 | −3.85 |
| At5g47850 | protein kinase, putative | −3.85 |
| At1g47400 | expressed protein | −3.86 |
| At5g44580 | expressed protein | −3.87 |
| At1g02930 | glutathione S-transferase, putative | −3.87 |
| At4g17670 | senescence-associated protein-related | −3.87 |
| At5g61900 | copine BONZAI1 (BON1) | −3.88 |
| At4g34390 | extra-large guanine nucleotide binding protein, putative/G-protein, | −3.88 |
| | putative | |
| At4g37560 | formamidase, putative/formamide amidohydrolase, putative | −3.88 |
| At1g22400 | UDP-glucoronosyl/UDP-glucosyl transferase family protein | −3.88 |
| At5g52740 | heavy-metal-associated domain-containing protein | −3.89 |
| At2g24600 | ankyrin repeat family protein | −3.90 |
| At5g46050 | proton-dependent oligopeptide transport (POT) family protein | −3.90 |
| psbK | — | −3.92 |
| At1g67520 | lectin protein kinase family protein | −3.93 |
| At4g14020 | rapid alkalinization factor (RALF) family protein | −3.95 |
| At1g72280 | endoplasmic reticulum oxidoreductin 1 (ERO1) family protein | −3.95 |
| At5g05300 | expressed protein | −3.97 |
| At5g53370 | pectinesterase family protein | −3.98 |
| At3g19030 | expressed protein | −3.98 |
| At3g12830 | auxin-responsive family protein | −3.98 |
| At2g18680 | expressed protein | −3.98 |
| At1g13210 | haloacid dehalogenase-like hydrolase family protein | −4.00 |
| At2g17290 | calcium-dependent protein kinase isoform 6 (CPK6) | −4.00 |
| At4g20110 | vacuolar sorting receptor, putative | −4.01 |
| At5g18670 | beta-amylase, putative (BMY3)/1,4-alpha-D-glucan maltohydrolase, | −4.03 |
| | putative | |
| At5g14930 | leaf senescence-associated protein (SAG101) | −4.04 |
| At5g58310 | hydrolase, alpha/beta fold family protein | −4.04 |
| At4g38560 | expressed protein | −4.04 |
| At3g49340 | cysteine proteinase, putative | −4.04 |
| At4g01010 | cyclic nucleotide-regulated ion channel, putative (CNGC13) | −4.06 |
| At1g78000 | sulfate transporter (Sultr1;2) | −4.08 |
| At3g44350 | no apical meristem (NAM) family protein | −4.10 |
| At2g47130 | short-chain dehydrogenase/reductase (SDR) family protein | −4.10 |
| At4g10120 | sucrose-phosphate synthase, putative | −4.10 |
| At3g57460 | expressed protein | −4.10 |
| At2g32160 | expressed protein | −4.11 |
| At2g13810 | aminotransferase class I and II family protein | −4.12 |
| At3g25610 | haloacid dehalogenase-like hydrolase family protein | −4.12 |
| At3g14470 | disease resistance protein (NBS-LRR class), putative | −4.14 |
| At1g56510 | disease resistance protein (TIR-NBS-LRR class), putative | −4.15 |
| At1g03870 | fasciclin-like arabinogalactan-protein (FLA9) | −4.16 |
| At5g10380 | zinc finger (C3HC4-type RING finger) family protein | −4.17 |
| At2g40140 | zinc finger (CCCH-type) family protein | −4.20 |
| At3g13950 | expressed protein | −4.22 |
| At5g42830 | transferase family protein | −4.23 |
| At2g46440 | cyclic nucleotide-regulated ion channel/cyclic nucleotide-gated channel | −4.26 |
| | (CNGC3) | |
| At4g08300 | nodulin MtN21 family protein | −4.26 |
| At4g14610 | disease resistance RPS2 protein | −4.29 |
| At2g30250 | WRKY family transcription factor | −4.31 |
| At5g48540 | 33 kDa secretory protein-related | −4.37 |
| At3g14770 | nodulin MtN3 family protein | −4.38 |
| At1g18570 | myb family transcription factor (MYB51) | −4.40 |
| At3g19850 | phototropic-responsive NPH3 family protein | −4.40 |
| At5g25930 | leucine-rich repeat family protein/protein kinase family protein | −4.41 |
| At4g01750 | expressed protein | −4.41 |
| At5g58940 | protein kinase family protein | −4.42 |
| At1g11260 | glucose transporter (STP1) | −4.42 |
| At5g40450 | expressed protein | −4.44 |
| At3g30775 | proline oxidase, mitochondrial/osmotic stress-responsive proline | −4.45 |
| | dehydrogenase (POX) (PRO1) (ERD5) | |
| At3g02840 | immediate-early fungal elicitor family protein | −4.46 |
| At4g16563 | aspartyl protease family protein | −4.47 |
| At1g74360 | leucine-rich repeat transmembrane protein kinase, putative | −4.48 |
| At5g53550 | transporter, putative | −4.48 |
| At4g31000 | calmodulin-binding protein | −4.48 |
| At4g28400 | protein phosphatase 2C, putative/PP2C, putative | −4.51 |
| At5g19240 | expressed protein | −4.53 |
| At2g15080 | disease resistance family protein | −4.57 |
| At2g30930 | expressed protein | −4.60 |
| At5g17760 | AAA-type ATPase family protein | −4.65 |
| At3g09940 | monodehydroascorbate reductase, putative | −4.66 |
| At1g15520 | ABC transporter family protein | −4.67 |
| At4g34150 | C2 domain-containing protein | −4.70 |
| At5g60800 | heavy-metal-associated domain-containing protein | −4.74 |
| At1g16420 | latex-abundant protein, putative (AMC8)/caspase family protein | −4.74 |
| At3g57520 | alkaline alpha galactosidase, putative | −4.74 |
| At1g19380 | expressed protein | −4.75 |
| At2g40000 | expressed protein | −4.80 |
| At2g29110 | glutamate receptor family protein (GLR2.8) (GLUR9) | −4.84 |
| At1g19250 | flavin-containing monooxygenase family protein/FMO family protein | −4.86 |
| At1g72240 | expressed protein | −4.87 |
| At2g36970 | UDP-glucoronosyl/UDP-glucosyl transferase family protein | −4.87 |
| At4g14400 | ankyrin repeat family protein | −4.89 |
| At5g44420 | plant defensin protein, putative (PDF1.2a) | −4.91 |
| At3g26220 | cytochrome P450 family protein | −4.92 |
| At4g02420 | lectin protein kinase, putative | −4.92 |
| At1g67800 | copine-related | −4.92 |
| At4g37370 | cytochrome P450, putative | −4.92 |
| At4g39830 | L-ascorbate oxidase, putative | −4.96 |
| At1g26380 | FAD-binding domain-containing protein | −4.99 |
| At1g76040 | calcium-dependent protein kinase, putative/CDPK, putative | −4.99 |
| At2g29120 | glutamate receptor family protein (GLR2.7) | −5.00 |
| At1g21250 | wall-associated kinase 1 (WAK1) | −5.02 |
| At1g21910 | AP2 domain-containing transcription factor family protein | −5.13 |
| At4g04220 | disease resistance family protein | −5.13 |
| At3g13100 | ABC transporter family protein | −5.19 |
| At2g26400 | acireductone dioxygenase (ARD/ARD′) family protein | −5.20 |
| At4g28490 | leucine-rich repeat transmembrane protein kinase, putative | −5.21 |
| At5g43910 | pfkB-type carbohydrate kinase family protein | −5.21 |
| At2g32800 | protein kinase family protein | −5.23 |
| At1g76970 | VHS domain-containing protein/GAT domain-containing protein | −5.23 |
| At3g23550 | MATE efflux family protein | −5.24 |
| At2g13790 | leucine-rich repeat family protein/protein kinase family protein | −5.31 |
| At5g60950 | phytochelatin synthetase-related | −5.33 |
| At5g24210 | lipase class 3 family protein | −5.35 |
| At1g13340 | expressed protein | −5.40 |
| At5g46230 | expressed protein | −5.46 |
| At2g47800 | glutathione-conjugate transporter (MRP4) | −5.50 |
| At5g52810 | ornithine cyclodeaminase/mu-crystallin family protein | −5.52 |
| At3g22060 | receptor protein kinase-related | −5.53 |
| At4g23610 | expressed protein | −5.56 |
| At5g58120 | disease resistance protein (TIR-NBS-LRR class), putative | −5.56 |
| At4g12720 | MutT/nudix family protein | −5.57 |
| At1g72900 | disease resistance protein (TIR-NBS class), putative | −5.59 |
| At5g10760 | aspartyl protease family protein | −5.60 |
| At1g64380 | AP2 domain-containing transcription factor, putative | −5.62 |
| At1g64500 | glutaredoxin family protein | −5.62 |
| At4g23200 | protein kinase family protein | −5.64 |
| At1g57560 | myb family transcription factor (MYB50) | −5.66 |
| At5g67340 | armadillo/beta-catenin repeat family protein/U-box domain-containing | −5.69 |
| | protein | |
| At1g66880 | serine/threonine protein kinase family protein | −5.73 |
| At1g56060 | expressed protein | −5.78 |
| At1g78290 | serine/threonine protein kinase, putative | −5.80 |
| At1g51890 | leucine-rich repeat protein kinase, putative | −5.82 |
| At2g26190 | calmodulin-binding family protein | −5.82 |
| At1g57990 | purine permease-related | −5.87 |
| At5g40690 | expressed protein | −5.91 |
| At3g52400 | syntaxin, putative (SYP122) | −5.96 |
| At4g28390 | ADP, ATP carrier protein, mitochondrial, putative/ADP/ATP | −5.99 |
| | translocase, putative | |
| At5g24530 | oxidoreductase, 2OG-Fe(II) oxygenase family protein | −6.00 |
| At4g33050 | calmodulin-binding family protein | −6.01 |
| At2g15390 | xyloglucan fucosyltransferase, putative (FUT4) | −6.01 |
| At5g11920 | glycosyl hydrolase family 32 protein | −6.05 |
| At4g23810 | WRKY family transcription factor | −6.05 |
| At1g78410 | VQ motif-containing protein | −6.06 |
| At2g04430 | MutT/nudix family protein | −6.09 |
| At5g22530 | expressed protein | −6.10 |
| At1g01340 | cyclic nucleotide-regulated ion channel (CNGC10) (ACBK1) | −6.11 |
| At2g46400 | WRKY family transcription factor | −6.14 |
| At2g26020 | plant defensin-fusion protein, putative (PDF1.2b) | −6.15 |
| At1g07000 | exocyst subunit EXO70 family protein | −6.17 |
| At2g02930 | glutathione S-transferase, putative | −6.18 |
| At1g24150 | formin homology 2 domain-containing protein/FH2 domain-containing | −6.21 |
| | protein | |
| At1g08450 | calreticulin 3 (CRT3) | −6.24 |
| At1g72060 | expressed protein | −6.28 |
| At3g45970 | expansin family protein (EXPL1) | −6.28 |
| At1g65490 | expressed protein | −6.31 |
| At1g75750 | gibberellin-regulated protein 1 (GASA1) | −6.33 |
| At3g23110 | disease resistance family protein | −6.34 |
| At5g54610 | ankyrin repeat family protein | −6.42 |
| At4g18250 | receptor serine/threonine kinase, putative | −6.54 |
| At5g57550 | xyloglucan:xyloglucosyl transferase (XTR3) | −6.54 |
| At3g07520 | glutamate receptor family protein (GLR1.4) | −6.62 |
| At3g24900 | disease resistance family protein/LRR family protein | −6.62 |
| At4g17030 | expansin-related | −6.63 |
| At5g26920 | calmodulin-binding protein | −6.66 |
| At1g74710 | isochorismate synthase 1 (ICS1)/isochorismate mutase | −6.78 |
| At2g14560 | expressed protein | −6.81 |
| At1g08050 | zinc finger (C3HC4-type RING finger) family protein | −6.85 |
| At4g04540 | protein kinase family protein | −6.86 |
| At3g26210 | cytochrome P450 71B23, putative (CYP71B23) | −6.94 |
| At3g48360 | speckle-type POZ protein-related | −6.95 |
| At4g25110 | latex-abundant family protein (AMC2)/caspase family protein | −7.00 |
| At3g48080 | lipase class 3 family protein/disease resistance protein-related | −7.08 |
| At5g01540 | lectin protein kinase, putative | −7.14 |
| At2g20145 | expressed protein | −7.19 |
| At3g51330 | aspartyl protease family protein | −7.22 |
| At3g11340 | UDP-glucoronosyl/UDP-glucosyl transferase family protein | −7.31 |
| At1g08930 | early-responsive to dehydration stress protein (ERD6)/sugar | −7.32 |
| | transporter family protein | |
| At5g54720 | ankyrin repeat family protein | −7.32 |
| At3g54150 | embryo-abundant protein-related | −7.33 |
| At1g58420 | expressed protein | −7.42 |
| At4g23310 | receptor-like protein kinase, putative | −7.43 |
| At2g30140 | UDP-glucoronosyl/UDP-glucosyl transferase family protein | −7.46 |
| At1g43910 | AAA-type ATPase family protein | −7.71 |
| At1g19020 | expressed protein | −7.80 |
| At4g34135 | UDP-glucoronosyl/UDP-glucosyl transferase family protein | −7.88 |
| At1g21240 | wall-associated kinase, putative | −7.97 |
| At1g66090 | disease resistance protein (TIR-NBS class), putative | −8.05 |
| At2g38470 | WRKY family transcription factor | −8.37 |
| At1g73805 | calmodulin-binding protein | −8.38 |
| At1g65480 | flowering locus T protein (FT) | −8.42 |
| At5g55450 | protease inhibitor/seed storage/lipid transfer protein (LTP) family protein | −8.50 |
| At4g13900 | pseudogene, similar to NL0D | −8.59 |
| At1g13470 | expressed protein | −8.67 |
| At1g33720 | cytochrome P450, putative | −8.68 |
| At2g41100 | touch-responsive protein/calmodulin-related protein 3, touch-induced | −8.85 |
| | (TCH3) | |
| At4g04490 | protein kinase family protein | −9.09 |
| At1g73800 | calmodulin-binding protein | −9.17 |
| At4g39670 | expressed protein | −9.27 |
| At2g24160 | pseudogene, leucine rich repeat protein family | −9.37 |
| At5g06530 | ABC transporter family protein | −9.72 |
| At3g48650 | pseudogene, At14a-related protein | −9.77 |
| At5g22380 | no apical meristem (NAM) family protein | −9.81 |
| At3g50480 | broad-spectrum mildew resistance RPW8 family protein | −10.43 |
| At2g31880 | leucine-rich repeat transmembrane protein kinase, putative | −10.46 |
| At3g52430 | phytoalexin-deficient 4 protein (PAD4) | −10.61 |
| At1g10340 | ankyrin repeat family protein | −10.89 |
| At1g01560 | mitogen-activated protein kinase, putative/MAPK, putative (MPK11) | −11.05 |
| At2g26560 | patatin, putative | −11.05 |
| At5g52750 | heavy-metal-associated domain-containing protein | −11.16 |
| At3g50930 | AAA-type ATPase family protein | −11.18 |
| At2g32680 | disease resistance family protein | −11.25 |
| At4g23220 | protein kinase family protein | −11.34 |
| At1g49200 | zinc finger (C3HC4-type RING finger) family protein | −11.49 |
| At5g41740 | disease resistance protein (TIR-NBS-LRR class), putative | −11.62 |
| At2g18690 | expressed protein | −11.74 |
| At5g66640 | LIM domain-containing protein-related | −12.13 |
| At1g57630 | disease resistance protein (TIR class), putative | −12.32 |
| At5g13320 | auxin-responsive GH3 family protein | −12.73 |
| At3g01290 | band 7 family protein | −12.90 |
| At3g26830 | cytochrome P450 71B15, putative (CYP71B15) | −12.92 |
| At3g45860 | receptor-like protein kinase, putative | −12.98 |
| At1g35230 | arabinogalactan-protein (AGP5) | −13.21 |
| At2g43570 | chitinase, putative | −13.28 |
| At3g13610 | oxidoreductase, 2OG-Fe(II) oxygenase family protein | −13.35 |
| At3g01830 | calmodulin-related protein, putative | −13.50 |
| At3g22600 | protease inhibitor/seed storage/lipid transfer protein (LTP) family protein | −13.54 |
| At5g37260 | myb family transcription factor | −14.15 |
| At4g14365 | zinc finger (C3HC4-type RING finger) family protein/ankyrin repeat | −14.15 |
| | family protein | |
| At1g33960 | avirulence-responsive protein/avirulence induced gene (AIG1) | −14.81 |
| At3g11010 | disease resistance family protein/LRR family protein | −15.66 |
| At3g57260 | glycosyl hydrolase family 17 protein | −15.68 |
| At1g30900 | vacuolar sorting receptor, putative | −15.75 |
| At4g03450 | ankyrin repeat family protein | −16.58 |
| At1g09080 | luminal binding protein 3 (BiP-3) (BP3) | −16.68 |
| At3g25010 | disease resistance family protein | −17.67 |
| At4g00700 | C2 domain-containing protein | −17.93 |
| At1g35710 | leucine-rich repeat transmembrane protein kinase, putative | −18.12 |
| At5g39670 | calcium-binding EF hand family protein | −18.46 |
| At4g04500 | protein kinase family protein | −18.76 |
| At5g64000 | 3′(2′),5′-bisphosphate nucleotidase, putative/inositol polyphosphate 1- | −19.35 |
| | phosphatase, putative | |
| At5g25260 | expressed protein | −19.56 |
| At3g60420 | expressed protein | −20.17 |
| At3g57240 | beta-1,3-glucanase (BG3) | −20.23 |
| At2g04450 | MutT/nudix family protein | −20.46 |
| At2g18660 | expansin family protein (EXPR3) | −20.82 |
| At4g02380 | late embryogenesis abundant 3 family protein/LEA3 family protein | −21.23 |
| At4g11890 | protein kinase family protein | −22.62 |
| At5g59670 | leucine-rich repeat protein kinase, putative | −25.03 |
| At5g18470 | curculin-like (mannose-binding) lectin family protein | −28.39 |
| At4g23140 | receptor-like protein kinase 5 (RLK5) | −30.43 |
| At4g23150 | protein kinase family protein | −34.62 |
| At1g75040 | pathogenesis-related protein 5 (PR-5) | −44.51 |
| At5g52760 | heavy-metal-associated domain-containing protein | −44.86 |
| At1g14870 | expressed protein | −45.29 |
| At4g10500 | oxidoreductase, 2OG-Fe(II) oxygenase family protein | −51.31 |
| At2g14610 | pathogenesis-related protein 1 (PR-1) | −54.95 |

TABLE 3
Common genes significantly up or down regulated 2-fold in Arabidopsis
overexpressing (OE) Arabidopsis GLK1 (AtGLK1) and wheat GLK1 (TaGLK1).
Data analysed using FlexArray for normalization and T-Test

| | | AtGLK1 | TaGLK1 |
|---|---|---|---|
| | | OE Fold | OE Fold |
| Transcript ID | Gene Title | Change | Change |
| At1g77960 | Phox-bem1 domain protein | 42.25 | 18.56 |
| At3g47340 | glutamine-dependent asparagine synthetase 1 (ASN1) | 12.29 | 5.24 |
| At2g39030 | GCN5-related N-acetyltransferase (GNAT) family protein | 10.94 | 3.70 |
| At4g23680 | major latex protein-related/MLP-related | 9.92 | 8.76 |
| At5g22500 | acyl CoA reductase, putative/male-sterility protein, putative | 8.42 | 3.32 |
| At5g45670 | GDSL-motif lipase/hydrolase family protein | 8.01 | 24.42 |
| At3g55970 | oxidoreductase, 2OG-Fe(II) oxygenase family protein | 8.01 | 7.33 |
| At5g37300 | expressed protein | 7.39 | 5.24 |
| At2g37770 | aldo/keto reductase family protein | 7.37 | 2.31 |
| At2g20670 | expressed protein | 6.28 | 2.43 |
| At1g13650 | expressed protein | 5.66 | 2.94 |
| At3g11480 | S-adenosyl-L-methionine:carboxyl methyltransferase family | 5.60 | 3.10 |
| | protein | | |
| At4g15210 | beta-amylase (BMY1)/1,4-alpha-D-glucan maltohydrolase | 5.40 | 2.25 |
| At3g16460 | jacalin lectin family protein | 5.15 | 3.91 |
| At1g73260 | trypsin and protease inhibitor family protein/Kunitz family | 4.95 | 3.57 |
| At2g23000 | serine carboxypeptidase S10 family protein | 4.84 | 6.04 |
| At1g56650 | myb family transcription factor (MYB75) | 4.67 | 3.90 |
| At5g48490 | protease inhibitor/seed storage/lipid transfer protein (LTP) | 4.49 | 2.50 |
| | family protein | | |
| At1g78170 | expressed protein | 4.47 | 2.10 |
| At1g14250 | nucleoside phosphatase family protein/GDA1/CD39 family | 4.37 | 3.14 |
| At3g15720 | glycoside hydrolase family 28 protein/polygalacturonase | 4.31 | 5.67 |
| | (pectinase) | | |
| At5g24780 | vegetative storage protein 2 (VSP2) | 4.20 | 2.05 |
| At3g04000 | short-chain dehydrogenase/reductase (SDR) family protein | 3.99 | 2.06 |
| At5g36910 | thionin (THI2.2) | 3.99 | 16.22 |
| At5g42800 | dihydroflavonol 4-reductase (dihydrokaempferol 4-reductase) | 3.74 | 12.64 |
| | (DFR) | | |
| At4g35060 | heavy-metal-associated domain-containing protein/copper | 3.73 | 2.71 |
| | chaperone (CCH)-related | | |
| At5g23820 | MD-2-related lipid recognition domain-containing protein/ML | 3.58 | 8.06 |
| | domain-containing protein | | |
| At1g35290 | thioesterase family protein | 3.55 | 2.29 |
| At1g53885 | senescence-associated protein-related | 3.54 | 3.60 |
| At2g47180 | galactinol synthase, putative | 3.49 | 2.93 |
| At5g59480 | haloacid dehalogenase-like hydrolase family protein | 3.47 | 2.15 |
| At2g15090 | fatty acid elongase, putative | 3.37 | 2.30 |
| At2g39920 | acid phosphatase class B family protein | 3.18 | 2.60 |
| At3g15950 | DNA topoisomerase-related | 3.11 | 12.88 |
| At2g34810 | FAD-binding domain-containing protein | 3.04 | 3.41 |
| At3g09260 | glycosyl hydrolase family 1 protein | 2.97 | 26.10 |
| At2g38870 | protease inhibitor, putative | 2.96 | 6.07 |
| At3g28220 | meprin and TRAF homology domain-containing protein/ | 2.92 | 2.82 |
| | MATH domain protein | | |
| At4g12500 | protease inhibitor/seed storage/lipid transfer protein (LTP) | 2.86 | 6.13 |
| | family protein | | |
| At2g21650 | myb family transcription factor | 2.81 | 4.24 |
| At3g28740 | cytochrome P450 family protein | 2.81 | 4.76 |
| At1g73870 | zinc finger (B-box type) family protein | 2.77 | 2.48 |
| At4g12490 | protease inhibitor/seed storage/lipid transfer protein (LTP) | 2.77 | 6.61 |
| | family protein | | |
| At5g24420 | glucosamine/galactosamine-6-phosphate isomerase-related | 2.69 | 11.34 |
| At2g43510 | trypsin inhibitor, putative | 2.69 | 3.27 |
| At1g16530 | LOB domain protein 3/lateral organ boundaries domain | 2.67 | 2.15 |
| | protein 3 (LBD3) | | |
| At2g22240 | inositol-3-phosphate synthase isozyme 2/myo-inositol-1- | 2.66 | 2.09 |
| | phosphate synthase 2/MI-1-P synthase 2/IPS 2 | | |
| At4g21910 | MATE efflux family protein | 2.66 | 2.14 |
| At5g24160 | squalene monooxygenase 1,2/squalene epoxidase 1,2 | 2.65 | 2.78 |
| | (SQP1,2) | | |
| At1g64200 | vacuolar ATP synthase subunit E, putative | 2.64 | 4.68 |
| At5g05600 | oxidoreductase, 2OG-Fe(II) oxygenase family protein | 2.59 | 2.19 |
| At4g39510 | cytochrome P450 family protein | 2.50 | 4.42 |
| At5g65020 | annexin 2 (ANN2) | 2.49 | 3.32 |
| At5g02940 | expressed protein | 2.45 | 2.09 |
| At2g02990 | ribonuclease 1 (RNS1) | 2.45 | 2.71 |
| At1g66760 | MATE efflux family protein | 2.44 | 3.10 |
| At5g17220 | glutathione S-transferase, putative | 2.44 | 7.26 |
| At2g39330 | jacalin lectin family protein | 2.44 | 4.08 |
| At1g62660 | beta-fructosidase (BFRUCT3)/beta-fructofuranosidase/ | 2.43 | 2.26 |
| | invertase, vacuolar | | |
| At4g17470 | palmitoyl protein thioesterase family protein | 2.43 | 9.67 |
| At5g44050 | MATE efflux family protein | 2.39 | 2.99 |
| At4g19430 | expressed protein | 2.39 | 10.34 |
| At4g22870 | leucoanthocyanidin dioxygenase, putative/anthocyanidin | 2.36 | 10.13 |
| | synthase, putative | | |
| At4g16730 | terpene synthase/cyclase family protein | 2.35 | 2.21 |
| At1g44350 | IAA-amino acid hydrolase 6, putative (ILL6)/IAA-Ala | 2.34 | 2.81 |
| | hydrolase, putative | | |
| At4g12480 | protease inhibitor/seed storage/lipid transfer protein (LTP) | 2.30 | 4.45 |
| | family protein | | |
| At1g55020 | lipoxygenase (LOX1) | 2.28 | 2.51 |
| At2g46640 | hypothetical protein | 2.26 | 2.35 |
| At2g43535 | trypsin inhibitor, putative | 2.25 | 2.73 |
| At1g22550 | proton-dependent oligopeptide transport (POT) family protein | 2.22 | 2.29 |
| At4g16590 | glucosyltransferase-related | 2.22 | 3.28 |
| At3g55110 | ABC transporter family protein | 2.20 | 4.53 |
| At5g15960 | stress-responsive protein (KIN1) | 2.19 | 2.69 |
| At4g11650 | osmotin-like protein (OSM34) | 2.17 | 3.85 |
| At1g56580 | expressed protein | 2.17 | 3.24 |
| At4g01080 | expressed protein | 2.16 | 2.49 |
| At1g75580 | auxin-responsive protein, putative | 2.16 | 2.29 |
| At5g54060 | glycosyltransferase family protein | 2.13 | 6.85 |
| At5g06870 | polygalacturonase inhibiting protein 2 (PGIP2) | 2.10 | 2.38 |
| At3g11900 | amino acid transporter family protein | 2.09 | 2.45 |
| At1g76790 | O-methyltransferase family 2 protein | 2.05 | 8.45 |
| At2g36870 | xyloglucan:xyloglucosyl transferase, putative | 2.02 | 2.47 |
| At5g54160 | quercetin 3-O-methyltransferase 1/flavonol 3-O- | 2.02 | 2.13 |
| | methyltransferase 1/caffeic acid/5-hydroxyferulic acid O- | | |
| | methyltransferase (OMT1) | | |
| At3g23410 | alcohol oxidase-related | 2.01 | 2.36 |
| At3g05180 | GDSL-motif lipase/hydrolase family protein | 2.01 | 3.03 |
| At5g65870 | phytosulfokines 5 (PSK5) | 2.01 | 2.02 |
| At5g13220 | expressed protein | 2.00 | 4.23 |
| At1g24150 | formin homology 2 domain-containing protein/FH2 domain- | −2.02 | −6.21 |
| | containing protein | | |
| At4g27300 | S-locus protein kinase, putative | −2.03 | −2.16 |
| At5g54610 | ankyrin repeat family protein | −2.06 | −6.42 |
| At2g36970 | UDP-glucoronosyl/UDP-glucosyl transferase family protein | −2.06 | −4.87 |
| At3g57260 | glycosyl hydrolase family 17 protein | −2.07 | −15.68 |
| At2g22500 | mitochondrial substrate carrier family protein | −2.07 | −3.42 |
| At1g35230 | arabinogalactan-protein (AGP5) | −2.07 | −13.21 |
| At2g41180 | sigA-binding protein-related | −2.07 | −3.12 |
| At1g03210 | phenazine biosynthesis PhzC/PhzF family protein | −2.08 | −2.35 |
| At3g09940 | monodehydroascorbate reductase, putative | −2.08 | −4.66 |
| At2g24850 | aminotransferase, putative | −2.10 | −2.59 |
| At5g60950 | phytochelatin synthetase-related | −2.11 | −5.33 |
| At1g76960 | expressed protein | −2.11 | −3.30 |
| At4g23130 | receptor-like protein kinase 6 (RLK6) | −2.12 | −2.10 |
| At2g26020 | plant defensin-fusion protein, putative (PDF1.2b) | −2.12 | −6.15 |
| At1g56120 | leucine-rich repeat family protein/protein kinase family protein | −2.14 | −2.17 |
| At1g08050 | zinc finger (C3HC4-type RING finger) family protein | −2.15 | −6.85 |
| At4g18250 | receptor serine/threonine kinase, putative | −2.16 | −6.54 |
| At1g01340 | cyclic nucleotide-regulated ion channel (CNGC10) (ACBK1) | −2.17 | −6.11 |
| At3g05650 | disease resistance family protein | −2.17 | −2.82 |
| At1g08450 | calreticulin 3 (CRT3) | −2.18 | −6.24 |
| At1g57630 | disease resistance protein (TIR class), putative | −2.18 | −12.32 |
| At4g14400 | ankyrin repeat family protein | −2.19 | −4.89 |
| At3g24900 | disease resistance family protein/LRR family protein | −2.21 | −6.62 |
| At2g14610 | pathogenesis-related protein 1 (PR-1) | −2.22 | −54.95 |
| At5g54720 | ankyrin repeat family protein | −2.23 | −7.32 |
| At3g50480 | broad-spectrum mildew resistance RPW8 family protein | −2.23 | −10.43 |
| At5g52810 | ornithine cyclodeaminase/mu-crystallin family protein | −2.23 | −5.52 |
| At5g25260 | expressed protein///expressed protein | −2.25 | −19.56 |
| At4g20780 | calcium-binding protein, putative | −2.25 | −2.59 |
| At5g44420 | plant defensin protein, putative (PDF1.2a) | −2.26 | −4.91 |
| At4g21830 | methionine sulfoxide reductase domain-containing protein/ | −2.26 | −2.97 |
| | SeIR domain-containing protein | | |
| At2g20870 | cell wall protein precursor, putative | −2.26 | −0.06 |
| At4g23200 | protein kinase family protein | −2.27 | −5.64 |
| At3g26210 | cytochrome P450 71B23, putative (CYP71B23) | −2.27 | −6.94 |
| At5g39670 | calcium-binding EF hand family protein | −2.27 | −18.46 |
| At3g60420 | expressed protein | −2.27 | −20.17 |
| At2g40750 | WRKY family transcription factor | −2.28 | −2.08 |
| At5g02760 | protein phosphatase 2C family protein/PP2C family protein | −2.28 | −3.78 |
| At1g10340 | ankyrin repeat family protein | −2.31 | −10.89 |
| At1g24020 | Bet v I allergen family protein | −2.31 | −0.10 |
| At3g51330 | aspartyl protease family protein | −2.32 | −7.22 |
| At5g58940 | protein kinase family protein | −2.34 | −4.42 |
| At1g35710 | leucine-rich repeat transmembrane protein kinase, putative | −2.34 | −18.12 |
| At5g45380 | sodium:solute symporter family protein | −2.35 | −3.55 |
| At4g11850 | phospholipase D gamma 1/PLD gamma 1 (PLDGAMMA1) | −2.37 | −2.81 |
| At4g00700 | C2 domain-containing protein | −2.39 | −17.93 |
| At4g34135 | UDP-glucoronosyl/UDP-glucosyl transferase family protein | −2.42 | −7.88 |
| At1g52200 | expressed protein | −2.42 | −2.91 |
| At4g23220 | protein kinase family protein | −2.46 | −11.34 |
| At2g04450 | MutT/nudix family protein | −2.47 | −20.46 |
| At4g23150 | protein kinase family protein | −2.50 | −34.62 |
| At1g21250 | wall-associated kinase 1 (WAK1) | −2.52 | −5.02 |
| At3g09020 | alpha 1,4-glycosyltransferase family protein/ | −2.54 | −2.97 |
| | glycosyltransferase sugar-binding DXD motif-containing | | |
| | protein | | |
| At2g02930 | glutathione S-transferase, putative | −2.55 | −6.18 |
| At3g48640 | expressed protein | −2.58 | −3.07 |
| At4g14365 | zinc finger (C3HC4-type RING finger) family protein/ankyrin | −2.61 | −14.15 |
| | repeat family protein | | |
| At3g01290 | band 7 family protein | −2.63 | −12.90 |
| At1g30900 | vacuolar sorting receptor, putative | −2.64 | −15.75 |
| At4g02380 | late embryogenesis abundant LEA 3 | −2.67 | −21.23 |
| At1g13470 | expressed protein | −2.71 | −8.67 |
| At3g23110 | disease resistance family protein | −2.73 | −6.34 |
| At5g59670 | leucine-rich repeat protein kinase, putative | -2.75 | -25.03 |
| At1g75040 | pathogenesis-related protein 5 (PR-5) | −2.76 | −44.51 |
| At4g01700 | chitinase, putative | −2.79 | −3.37 |
| At1g77510 | protein disulfide isomerase, putative | −2.82 | −2.91 |
| At3g25010 | disease resistance family protein | −2.90 | −17.67 |
| At4g23140 | receptor-like protein kinase 5 (RLK5) | −2.95 | −30.43 |
| At2g47130 | short-chain dehydrogenase/reductase (SDR) family protein | −2.97 | −4.10 |
| At4g11890 | protein kinase family protein | −3.03 | −22.62 |
| At4g10500 | oxidoreductase, 2OG-Fe(II) oxygenase family protein | −3.17 | −51.31 |
| At5g52760 | heavy-metal-associated domain-containing protein | −3.20 | −44.86 |
| At3g48080 | lipase class 3 family protein/disease resistance protein- | −3.31 | −7.08 |
| | related | | |
| At5g52740 | heavy-metal-associated domain-containing protein | −3.31 | −3.89 |
| At5g59680 | leucine-rich repeat protein kinase, putative | −3.34 | −2.50 |
| At4g17030 | expansin-related | −3.35 | −6.63 |
| At3g16530 | legume lectin family protein | −3.57 | −3.65 |
| At4g03450 | ankyrin repeat family protein | −3.60 | −16.58 |
| At2g26560 | patatin, putative | −3.60 | −11.05 |
| At3g23550 | MATE efflux family protein | −3.61 | −5.24 |
| At1g14870 | expressed protein | −3.73 | −45.29 |
| At1g67810 | Fe—S metabolism associated domain-containing protein | −3.77 | −2.41 |
| At3g11010 | disease resistance family protein/LRR family protein | −3.82 | −15.66 |
| At5g55450 | protease inhibitor/seed storage/lipid transfer protein (LTP) | −3.90 | −8.50 |
| | family protein | | |
| At2g14560 | expressed protein | −3.96 | −6.81 |
| At1g09080 | luminal binding protein 3 (BiP-3) (BP3) | −4.12 | −16.68 |
| At4g25110 | latex-abundant family protein (AMC2)/caspase family protein | −4.21 | −7.00 |
| At4g08300 | nodulin MtN21 family protein | −4.23 | −4.26 |
| At5g18470 | curculin-like (mannose-binding) lectin family protein | −4.23 | −28.39 |
| At1g29720 | protein kinase family protein | −5.41 | −2.26 |
| At3g57240 | beta-1,3-glucanase (BG3) | −5.50 | −20.23 |

### Example 5

### Identification of GLK1 Regulated Genes

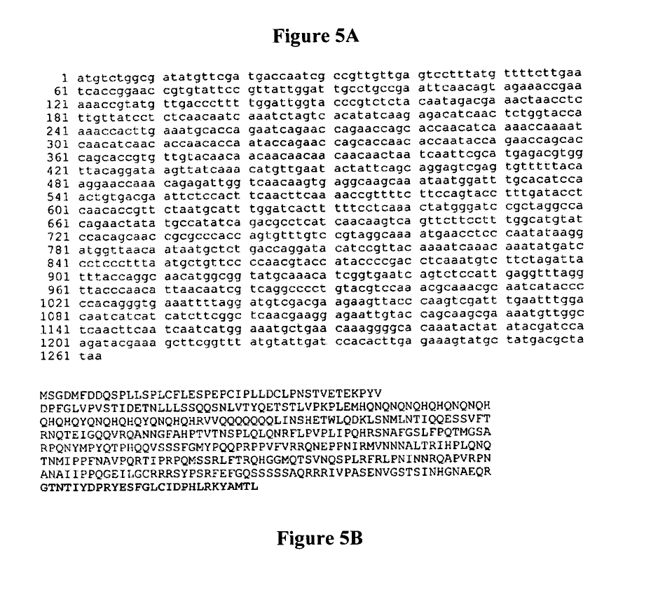

As can be seen from FIG. 4, and Table 1, one of the most highly accumulated transcripts encode At1g77960 (FIGS. 5A-5B), a hypothetical protein of unknown function. At1s77960 encodes a protein homologous to the octicosapeptide/Phox/Bem1 p (PB1) domain-containing protein At5g09620.1 and D. discoideum GB:EAL72846.1. A PB1 domain containing protein has been observed to be upregulated in response to the pathogen C. higgensianum in Arabidopsis (Narusaka et al, 2004). PB1 domain containing proteins are observed in MEK kinases (Nakamura and Johnson, 2003) and in activation of NADP oxidase in response to oxidative stress (Ito et al, 2001).

### Example 6

### Isolation of Wheat GLK1 (TaGLK1)

A full length cDNA of wheat GLK1 (Genbank Accession ABL10089) was isolated by screening ESTs generated from a bread wheat cDNA library challenged with the stem rust pathogen. The sequence of rice GLK1 (OSGLK1, accession AAK50393) was used to screen the wheat ESTs. TaLr1168E10R, dbEST Gen Bank accession no. BQ620355 was identified.

The full length wheat GLK1 (TaGLK1, accession ABL10089) was cloned from wheat mRNA using RT-PCR primers derived from the sequence of TaLr1168E10R. HindIII and EcoRV sites were created on the complete coding region of TaGLK1 (Genbank accession: ABL 10089) using the following primers:

(SEQ ID NO: 44)
TaGLK1 5′ primer-cgtaagcttggctgtggtgggtag
(SEQ ID NO: 45)
TaGLK1 3′ primer-ttgttgtcgatatcgtgctgctgcta.

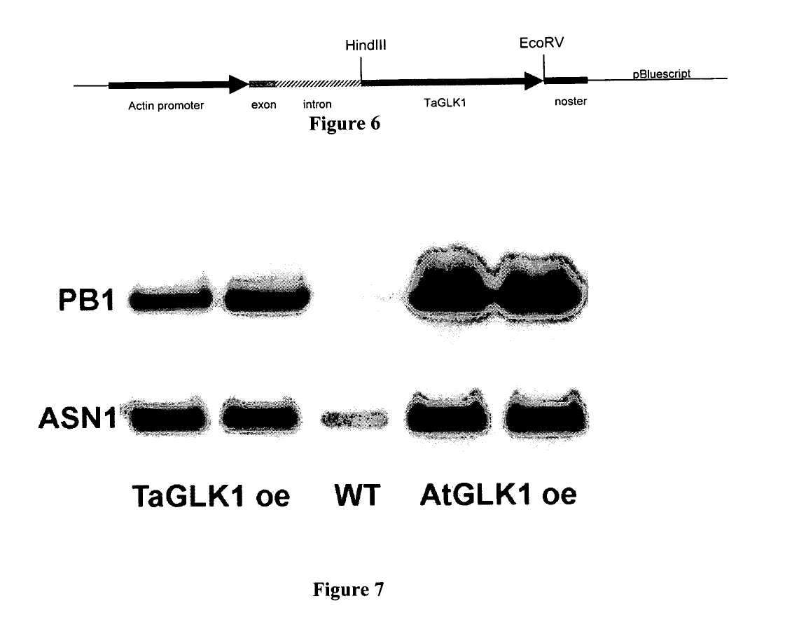

The complete coding region of TaGLK1 (Genbank accession: ABL10089) was inserted into the bombardment vector pAct-nos (a gift of J. Simmonds, Agriculture and Agri-Food Canada) containing the rice Actin1 promoter and intron (D McElroy, W Zhang, J Cao, and R Wu, 1900 Plant Cell 2: 193-171) to form pAct-TaGLK1 as indicated in FIG. 6.

Transformation into wheat cv. Fielder by particle bombardment is carried out as described by Jordan (Plant Cell Reports 19:1069-1075, 2000).

Examples of wheat plants expressing TaGLK1 is shown in FIG. 8.

### Example 7

### Transformation of Arabidopsis with Wheat GLK1 (TaGLK1)

TaGLK1 was inserted into the BamHI and KpnI sites of transformation vector pHS723-TaGLK1, introduced into Agrobacterium tumafaciens strain GV3109 pmp90 and used to transform Arabidopsis as describe for AtGLK1 (see Example 2) using the method described in Savitch et al, (2007, Biochem Biophys Res Comm 359: 234-238).

Transformation of Brassica napus with Arabidopsis GLK1 (AtGLK1):

B. napus cv. Westar was transformed with pHS723-AtGLK1 as described in Savitch et al, 2005, Plant Cell Physiol 46: 1525-1539).

Transformation of Maize with AtGLK1:

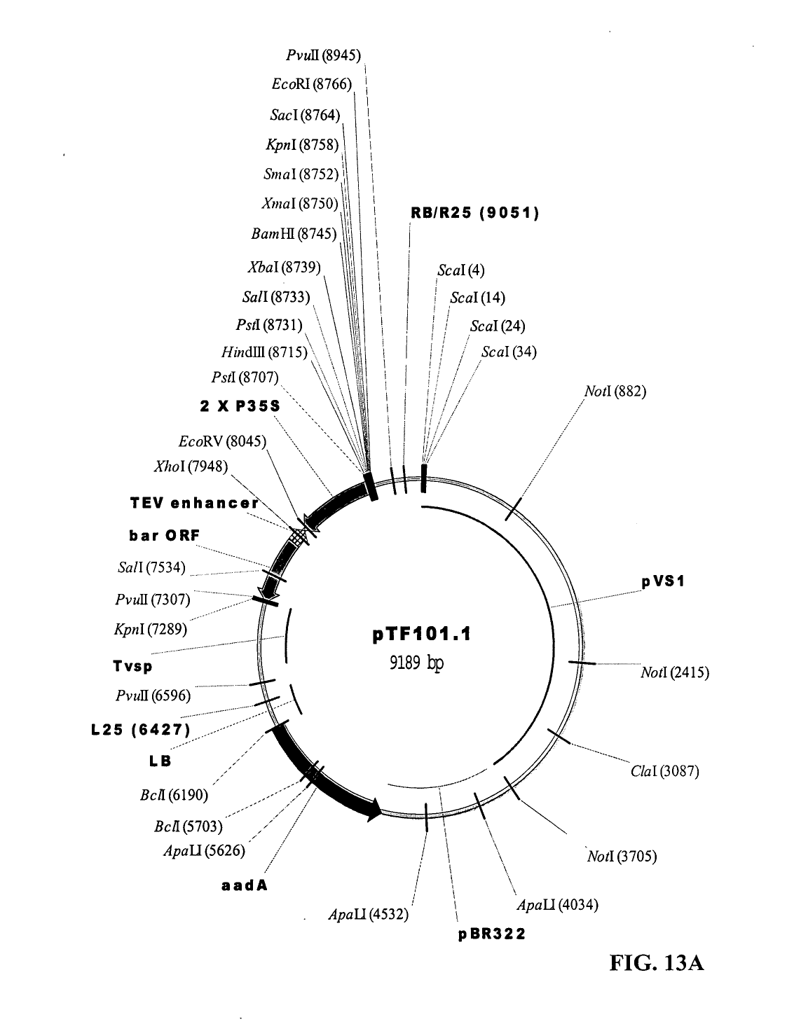

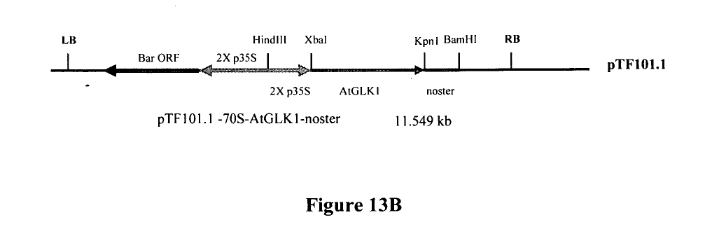

AtGLK1, driven by the 2× CaMV 35S promoter, was inserted into the HindIII and BamH1 sites of pTF 101.1 (FIG. 13A) to produce the transformation vector pTF101.1-70S-AtGLK1-noster (FIG. 13B). Agrobacterium tumifaciens (strain EHA101) mediated transformation (Frame et al, 2002, Plant Physiol 129: 14-22) was used to introduce pTF101.1-70S-AtGLK1-poster into F2 immature embryos of the maize hybrid genotype Hi II.

Transformation of Rice with AtGLK1:

AtGLK1, driven by the 2× CaMV 35S promoter, was inserted into the HindIII and BamH1 sites of pTF 101 1 (FIG. 13A) to produce the transformation vector pTF101.1-70S-AtGLK1-noster (FIG. 13B). Agrobacterium tumifaciens (strain EHA101) transformed with pTF101.1-70S-AtGLK1 were introduced into mature embryo derived callus tissues of Oryza japonicum cv. Nipponbare (Hiei et al, 1994, The Plant J 6: 271-282; Toki, 1997, Plant Mol Biology Rep 15:16-21). Transformed calli were selected on 2 mg/L Bialaphos.

### Soybean Transformation:

AtGLK1, driven by the 2× CaMV 35S promoter, was inserted into the HindIII and BamH1 sites of pTF 101.1 (FIG. 13A) to produce the transformation vector pTF101.1-70S-AtGLK1-noster (FIG. 13B). Agrobacterium tumifaciens (strain EHA 101) transformed with pTF 101.1-70S-AtGLK1 were introduced into explants of the cultivar Williams 82. Plantlets regenerated from cocultivated explants were screened with Liberty herbicide for transformants (Paz et al, 2006, Plant Cell Rep 25: 248-255).

### Example 8

### Infection of Wheat Leaves with F. graminearum

Individual wheat seedlings (6-8 inch first leaf) grown on soil were removed from the soil and placed horizontally between two glass rods in a transparent chamber. The roots were covered with a moist filter paper connected by a wick to a small beaker in the chamber to prevent drying. The leaves were infected by lightly pressing a glass Pasteur pipette tip on the axial leaf surface and releasing inoculum containing F. graminearum (50,000 spores/ml) on the leaf surface. The chamber was sealed and placed in the dark for 24 h, then returned to an environmental growth chamber set at 25° C., 16 h light period at 200 μEm⁻². The spread of the lesions were compared between different genotypes from days 1-8. Mock inoculation with water alone were also performed. Results are shown in FIG. 9.

### Example 9

### Infection of Wheat Heads with Fusarium graminearum

Maturing wheat heads at 50% anthesis were spray individually with 5 ml of F. graminearum inoculum containing 50,000 macroconidia spores/ml. The sprayed plants were placed in a Conviron E15 environmental chamber equipped with a fine water mist that is activated at 30 min intervals and set at 25° C., 16 h light at 200 μEm⁻². Browning of individual florets was recorded daily. Examples of wheat heads following treatment are shown in FIG. 10.

Transgenic wheat (Triticum aestivum cv Fielder) over-expressing wheat TaGLK1 (FIG. 8) shows enhanced resistance to the spread of lesions in wheat leaves inoculated with F. graminearum (FIG. 9) and in the development of Fusarium Head Blight (FHB) symptoms in developing wheat heads (FIG. 10)

### REFERENCES

Savitch L V, Allard G, Seki M, Robert L S, Tinker N A, Huner N P, Shinozaki K, Singh J. The effect of overexpression of two Brassica CBF/DREB1-like transcription factors on photosynthetic capacity and freezing tolerance in Brassica napus. Plant Cell Physiol. 2005 September; 46(9):1525-39. Epub 2005 Jul. 16.
Ramesh B. Nair, Richard W. Joy I V, Eugen Kurylo, Xiaohong Shi, Joan Schnaider, Raju S. S. Datla, Wilf A. Keller, and Gopalan Selvaraj. Identification of a CYP84 Family of Cytochrome P450-Dependent Mono-Oxygenase Genes in Brassica napus and Perturbation of Their Expression for Engineering Sinapine Reduction in the Seeds. Plant Physiol, August 2000, Vol. 123, pp. 1623-1634.
Clough S J, and Bent A F. Floral dip: a simplified method for Agrobacterium-mediated transformation of Arabidopsis thaliana. Plant J. 1998 December; 16(6):735-43.
Makandar R, Essig J S, Schapaugh M A, Trick H N, Shah J, (2006) Genetically engineered resistance to Fusarium Head Blight in wheat by expression of Arabidopsis NPR1. Mol Plant-Microb Inter 19:123-129

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A method of conferring resistance to a pathogen in a plant, the method comprising:
i) introducing a nucleic acid molecule encoding a GLK1 polypeptide or fragment or variant thereof into a plant cell to produce a transgenic plant cell, such that the nucleic acid molecule is positioned for expression in the transgenic plant cell; and
ii) growing a transgenic plant from the transgenic plant cell,
wherein the GLK1 polypeptide or fragment or variant thereof is over-expressed relative to wild type GLK1 at least 2 fold, whereby the 2 fold over-expression is sufficient to confer resistance to the pathogen in the plant.

2. The method of claim 1 wherein the nucleic acid molecule comprises a nucleic acid sequence substantially identical to the nucleic acid sequence set forth in SEQ ID NO: 1.

3. The method of claim 1 wherein the nucleic acid molecule comprises the nucleic acid sequence set forth in SEQ ID NO: 1.

4. The method of claim 1 wherein the nucleic acid molecule hybridizes under conditions of high stringency to the nucleic acid sequence set forth in SEQ ID NO: 1.

5. The method of claim 1 wherein the GLK1 polypeptide comprises an amino acid sequence substantially identical to the amino acid sequence set forth in SEQ ID NO: 2.

6. The method of claim 1 wherein the GLK1 polypeptide is an Arabidopsis polypeptide.

7. The method of claim 1 wherein the GLK1 polypeptide is selected from the group consisting of a wheat GLK1 polypeptide, a maize GLK1 polypeptide, a rice GLK1 polypeptide, and a soy bean GLK1 polypeptide.

8. The method of claim 1 wherein the plant is selected from the group consisting of canola, Brassica spp., maize, tobacco, alfalfa, potato, ginseng, pea, oat, rice, soybean, wheat, barley, sunflower, and cotton.

9. The method of claim 1 wherein the pathogen is selected from the group consisting of Fusarium graminearum, Sclerotinia sclerotium, Puccinia graminis, Puccinia triticinia, Puccinia recondite, and Phytopthora infestans.

10. A genetic construct comprising a regulatory region operably linked to a nucleic acid molecule encoding a GLK1 polypeptide or fragment or variant thereof.

11. The genetic construct of claim 10 wherein the nucleic acid molecule comprises a nucleic acid sequence substantially identical to the nucleic acid sequence set forth in SEQ ID NO: 1.

12. The genetic construct of claim 10 wherein the nucleic acid molecule comprises the nucleic acid sequence set forth in SEQ ID NO: 1.

13. The genetic construct of claim 10 wherein the nucleic acid molecule hybridizes under conditions of high stringency to the nucleic acid sequence set forth in SEQ ID NO: 1.

14. The genetic construct of claim 10 wherein the GLK1 polypeptide is selected from the group consisting of a wheat GLK1 polypeptide, a maize GLK1 polypeptide, a rice GLK1 polypeptide, and a soy bean GLK1 polypeptide.

15. A pathogen resistant plant comprising a GLK1 nucleic acid molecule that encodes a GLK1 polypeptide, wherein expression of the GLK1 produces at least 2 fold greater GLK1 polypeptide relative to a wild-type plant.

16. A vector comprising the genetic construct of claim 10.

17. A host cell, a transgenic plant or a transgenic seed comprising the genetic construct of claim 10.

18. The host cell, transgenic plant, or transgenic seed of claim 17 wherein the host cell, transgenic plant or transgenic seed is selected from the group consisting of canola, Brassica spp., maize, tobacco, alfalfa, potato, ginseng, pea, oat, rice, soybean, wheat, barley, sunflower, and cotton.

19. A method of increasing the nitrogen status in a plant, the method comprising:
i) introducing a nucleic acid molecule encoding a GLK1 polypeptide or fragment or variant thereof into a plant cell to produce a transgenic plant cell, such that the nucleic acid molecule is positioned for expression in the transgenic plant cell; and
ii) growing a transgenic plant from the transgenic plant cell,
wherein the GLK1 polypeptide or fragment or variant thereof is over-expressed relative to wild type GLK1 at least 2 fold, whereby the 2 fold over-expression is sufficient to confer resistance to the pathogen in the plant.

20. A plant, or a plant cell with increased nitrogen status, the plant or plant cell comprising a GLK1 nucleic acid molecule that encodes a GLK1 polypeptide, wherein expression of the GLK1 produces at least 2 fold greater GLK1 polypeptide relative to a wild-type plant. the increase in nitrogen status determined by comparing the plant or plant cell with a plant or plant cell that does not express the GLK1 nucleic acid molecule and determining the levels of expression of ASN1, the colour of the leaves, or both, wherein an increase in the expression of ASN1, a darker green leaf, or both, when compared to the plant that does not express GLK1, indicates increased nitrogen status.


SEQUENCE LISTING (57 sequences)

SEQ ID NO: 1 - DNA, 1625 bp, Artificial Sequence
  Description of Artificial Sequence; note = synthetic construct
gaagaaagagacttactttggatatcaaaacatgtcttgctctctattctaattaagtctagcctatctacaacatacataaacctctatctacgattatatttggatctacaagtgaagattgatcgatgttagctctgtctccggcgacaagagatggttgcgacggagcgtcagagtttcttgatacgtcgtgtggattcacgattataaacccggaggaggaggaggagtttccggatttcgctgaccacggtgatcttcttgacatcattgacttcgacgatatattcggtgtggccggagatgtgcttcctgacttggagatcgaccctgagatcttatccggggatttctccaatcacatgaacgcttcttcaacgattactacgacgtcggataagactgatagtcaaggggagactactaagggtagttcggggaaaggtgaagaagtcgtaagcaaaagagacgatgttgcggcggagacggtgacttatgacggtgacagtgaccggaaaaggaagtattcctcttcagcttcttccaagaacaatcggatcagtaacaacgaagggaagagaaaggtgaaggtggattggacaccagagctacacaggagattcgtggaggcagtggaacagttaggagtggacaaagctgttccttctcgaattctggagcttatgggagtccattgtctcactcgtcacaacgttgctagtcacctccaaaaatataggtctcatcggaaacatttgctagctcgtgaggccgaagcggctaattggacacgcaaaaggcatatctatggagtagacaccggtgctaatcttaatggtcggactaaaaatggatggcttgcaccggcacccactctcgggtttccaccaccaccacccgtggctgttgcaccgccacctgtccaccaccatcattttaggcccctgcatgtgtggggacatcccacggttgatcagtccattatgccgcatgtgtggcccaaacacttacctccgccttctaccgccatgcctaatccgccgttttgggtctccgattctccctattggcatccaatgcataacgggacgactccgtatttaccgaccgtagctacgagatttagagcaccgccagttgccggaatcccgcatgctctgccgccgcatcacacgatgtacaaaccaaatcttggatttggtggtgctcgtcctccggtagacttacatccgtcaaaagagagcgtggatgcagccataggagatgtattgacgaggccatggctgccacttccgttgggattaaatccgccggctgttgacggtgttatgacagagcttcaccgtcacggtgtctctgaggttcctccgaccgcgtcttgtgcctgaaacgcacaagatccgtaggcaagcgagaaccaaacaaaaattcgacgacatgtctttcaattattgtacttttatttttccaagggttgtttttgttttgacccttgttgtgttaagagttactctttacttaatcgtcccatgtgactaataaggagacccttttcatatgtatctagggtgctaagtacactctagctctattaacgcaaataaattacctatattttctct

SEQ ID NO: 2 - protein, 420 aa, Artificial Sequence
  Description of Artificial Sequence; note = synthetic construct
MLALSPATRDGCDGASEFLDTSCGFTIINPEEEEEFPDFADHGDLLDIIDFDDIFGVAGDVLPDLEIDPEILSGDFSNHMNASSTITTTSDKTDSQGETTKGSSGKGEEVVSKRDDVAAETVTYDGDSDRKRKYSSSASSKNNRISNNEGKRKVKVDWTPELHRRFVEAVEQLGVDKAVPSRILELMGVHCLTRHNVASHLQKYRSHRKHLLAREAEAANWTRKRHIYGVDTGANLNGRTKNGWLAPAPTLGFPPPPPVAVAPPPVHHHHFRPLHVWGHPTVDQSIMPHVWPKHLPPPSTAMPNPPFWVSDSPYWHPMHNGTTPYLPTVATRFRAPPVAGIPHALPPHHTMYKPNLGFGGARPPVDLHPSKESVDAAIGDVLTRPWLPLPLGLNPPAVDGVMTELHRHGVSEVPPTASCA

SEQ ID NO: 3 - DNA, 1263 bp, Artificial Sequence
  Description of Artificial Sequence; note = synthetic construct
atgtctggcgatatgttcgatgaccaatcgccgttgttgagtcctttatgttttcttgaatcaccggaaccgtgtattccgttattggattgcctgccgaattcaacagtagaaaccgaaaaaccgtatgttgacccttttggattggtacccgtctctacaatagacgaaactaacctcttgttatcctctcaacaatcaaatctagtcacatatcaagagacatcaactctggtaccaaaaccacttgaaatgcaccagaatcagaaccagaaccagcaccaacatcaaaaccaaaatcaacatcaacaccaacaccaataccagaaccagcaccaacaccaataccagaaccagcaccagcaccgtgttgtacaacaacaacaacaacaacaactaatcaattcgcatgagacgtggttacaggataagttatcaaacatgttgaatactattcagcaggagtcgagtgtttttacaaggaaccaaacagagattggtcaacaagtgaggcaagcaaataatggatttgcacatccaactgtgacgaattctccacttcaacttcaaaaccgttttcttccagtacctttgatacctcaacaccgttctaatgcatttggatcactttttcctcaaactatgggatccgctaggccacagaactatatgccatatcagacgcctcatcaacaagtcagttcttcctttggcatgtatccacagcaaccgcgcccaccagtgtttgtccgtaggcaaaatgaacctcccaatataaggatggttaacaataatgctctgaccaggatacatccgttacaaaatcaaacaaatatgatccctccctttaatgctgttccccaacgtaccataccccgacctcaaatgtcttctagattatttaccaggcaacatggcggtatgcaaacatcggtgaatcagtctccattgaggtttaggttacccaacattaacaatcgtcaggcccctgtacgtccaaacgcaaacgcaatcatacccccacagggtgaaattttaggatgtcgacgaagaagttacccaagtcgatttgaatttggacaatcatcatcatcttcggctcaacgaaggagaattgtaccagcaagcgaaaatgttggctcaacttcaatcaatcatggaaatgctgaacaaaggggcacaaatactatatacgatccaagatacgaaagcttcggtttatgtattgatccacacttgagaaagtatgctatgacgctataa

SEQ ID NO: 4 - protein, 420 aa, Artificial Sequence
  Description of Artificial Sequence; note = synthetic construct
MSGDMFDDQSPLLSPLCFLESPEPCIPLLDCLPNSTVETEKPYVDPFGLVPVSTIDETNLLLSSQQSNLVTYQETSTLVPKPLEMHQNQNQNQHQHQNQNQHQHQHQYQNQHQHQYQNQHQHRVVQQQQQQQLINSHETWLQDKLSNMLNTIQQESSVFTRNQTEIGQQVRQANNGFAHPTVTNSPLQLQNRFLPVPLIPQHRSNAFGSLFPQTMGSARPQNYMPYQTPHQQVSSSFGMYPQQPRPPVFVRRQNEPPNIRMVNNNALTRIHPLQNQTNMIPPFNAVPQRTIPRPQMSSRLFTRQHGGMQTSVNQSPLRFRLPNINNRQAPVRPNANAIIPPQGEILGCRRRSYPSRFEFGQSSSSSAQRRRIVPASENVGSTSINHGNAEQRGTNTIYDPRYESFGLCIDPHLRKYAMTL

SEQ ID NO: 5 - DNA, 23 bp, Artificial Sequence
  Description of Artificial Sequence; note = synthetic construct
tatatttggatctagaagtgaag

SEQ ID NO: 6 - DNA, 21 bp, Artificial Sequence
  Description of Artificial Sequence; note = synthetic construct
ttggttctcgggtacctacgg

SEQ ID NO: 7 - DNA, 23 bp, Artificial Sequence
  Description of Artificial Sequence; note = synthetic construct
tcggggaaaggtgaagaagtcgt

SEQ ID NO: 8 - DNA, 22 bp, Artificial Sequence
  Description of Artificial Sequence; note = synthetic construct
cagccatggcctcgtcaataca

SEQ ID NO: 9 - DNA, 24 bp, Artificial Sequence
  Description of Artificial Sequence; note = synthetic construct
atggcacctgagatgaacaacaag

SEQ ID NO: 10 - DNA, 24 bp, Artificial Sequence
  Description of Artificial Sequence; note = synthetic construct
gcaaacatataaggtacaaagagg

SEQ ID NO: 11 - DNA, 21 bp, Artificial Sequence
  Description of Artificial Sequence; note = synthetic construct
caaccacatgcacactaattc

SEQ ID NO: 12 - DNA, 21 bp, Artificial Sequence
  Description of Artificial Sequence; note = synthetic construct
ccattggctgaaggagtctcg

SEQ ID NO: 13 - DNA, 22 bp, Artificial Sequence
  Description of Artificial Sequence; note = synthetic construct
caacatgcctccaaagccactc

SEQ ID NO: 14 - DNA, 24 bp, Artificial Sequence
  Description of Artificial Sequence; note = synthetic construct
acgcatacaataactctcccacac

SEQ ID NO: 15 - DNA, 20 bp, Artificial Sequence
  Description of Artificial Sequence; note = synthetic construct
ctatcaagccgcctcaccta

SEQ ID NO: 16 - DNA, 18 bp, Artificial Sequence
  Description of Artificial Sequence; note = synthetic construct
ctcaccgacccgccagta

SEQ ID NO: 17 - DNA, 24 bp, Artificial Sequence
  Description of Artificial Sequence; note = synthetic construct
atcgccgttgttgagtcctttatg

SEQ ID NO: 18 - DNA, 24 bp, Artificial Sequence
  Description of Artificial Sequence; note = synthetic construct
agtatttgtgcccctttgttcagc

SEQ ID NO: 19 - DNA, 18 bp, Artificial Sequence
  Description of Artificial Sequence; note = synthetic construct
gatggcgcgtggttgttt

SEQ ID NO: 20 - DNA, 19 bp, Artificial Sequence
  Description of Artificial Sequence; note = synthetic construct
ggggaggctatcgcagttg

SEQ ID NO: 21 - DNA, 24 bp, Artificial Sequence
  Description of Artificial Sequence; note = synthetic construct
catctctagtactgcctcttctcc

SEQ ID NO: 22 - DNA, 23 bp, Artificial Sequence
  Description of Artificial Sequence; note = synthetic construct
tgtgtaaccaatatcccaaaatg

SEQ ID NO: 23 - DNA, 18 bp, Artificial Sequence
  Description of Artificial Sequence; note = synthetic construct
tgcctcggcgtttcagta

SEQ ID NO: 24 - DNA, 19 bp, Artificial Sequence
  Description of Artificial Sequence; note = synthetic construct
tctccgacgaccgatttct

SEQ ID NO: 25 - DNA, 18 bp, Artificial Sequence
  Description of Artificial Sequence; note = synthetic construct
cgccgcaccgctcctctg

SEQ ID NO: 26 - DNA, 24 bp, Artificial Sequence
  Description of Artificial Sequence; note = synthetic construct
gcggtacctaacgtcggctttctc

SEQ ID NO: 27 - DNA, 23 bp, Artificial Sequence
  Description of Artificial Sequence; note = synthetic construct
agtgccccaggtcgagtttgatg

SEQ ID NO: 28 - DNA, 24 bp, Artificial Sequence
  Description of Artificial Sequence; note = synthetic construct
tcccgcctttctttagttgattgg

SEQ ID NO: 29 - DNA, 19 bp, Artificial Sequence
  Description of Artificial Sequence; note = synthetic construct
ctcaagatagcccacaaga

SEQ ID NO: 30 - DNA, 19 bp, Artificial Sequence
  Description of Artificial Sequence; note = synthetic construct
aaggccacatattttacat

SEQ ID NO: 31 - DNA, 23 bp, Artificial Sequence
  Description of Artificial Sequence; note = synthetic construct
tcggggaaaggtgaagaagtcgt

SEQ ID NO: 32 - DNA, 22 bp, Artificial Sequence
  Description of Artificial Sequence; note = synthetic construct
cagccatggcctcgtcaataca

SEQ ID NO: 33 - DNA, 2192 bp, Artificial Sequence
  Description of Artificial Sequence; note = synthetic construct
ctcatcttctcttcttcttcccccagtcctccccccctttcccctcttcggcctctctcgctcagctcactcttcattaagcgagctcacgtcgttcctcccttcttcttcttcttatccgttgttcaattcgttcagctagccggccagagatcgagcatcatctccatcatcgattcatccatctcatctttctcttcttctattctatgtcgtcgtcgtcccagattagatcgaagctgctagcagtctatccagtctctagctagctagctagatcaagcccgcagactatacaatataatacaagctagctacgtgcttattattgctttattagtctagaataatcttgatatatacgatcgaacatatatctcgatctccaccgatacacacccggccctatccatgcttgaggtgtcgacgctgcgcggccctactagcagcggcagcaaggcggagcagcactgcggcggcggcggcggcttcgtcggcgaccaccatgtggtgttcccgacgtccggcgactgcttcgccatggtggacgacaacctcctggactacatcgacttcagctgcgacgtgcccttcttcgacgctgacggggacatcctccccgacctggaggtagacaccacggagctcctcgccgagttctcgtccacccctcctgcggacgacctgctggcagtggcagtattcggcgccgacgaccagccggcggcggcagtagcacaagagaagccgtcgtcgtcgttggagcaaacatgtggtgacgacaaaggtgtagcagtagccgccgccagaagaaagctgcagacgacgacgacgacgacgacgacggaggaggaggattcttctcctgccgggtccggggccaacaagtcgtcggcgtcggcagagggccacagcagcaagaagaagtcggcgggcaagaactccaacggcggcaagcgcaaggtgaaggtggactggacgccggagctgcaccggcggttcgtgcaggcggtggagcagctgggcatcgacaaggccgtgccgtccaggatcctggagatcatgggcacggactgcctcacaaggcacaacattgccagccacctccagaagtaccggtcgcacagaaagcacctgatggcgcgggaggcggaggccgccacctgggcgcagaagcgccacatgtacgcgccgccagctccaaggacgacgacgacgacggacgccgccaggccgccgtgggtggtgccgacgaccatcgggttcccgccgccgcgcttctgccgcccgctgcacgtgtggggccacccgccgccgcacgccgccgcggctgaagcagcagcggcgactcccatgctgcccgtgtggccgcgtcacctggcgccgccccggcacctggcgccgtgggcgcacccgacgccggtggacccggcgttctggcaccagcagtacagcgctgccaggaaatggggcccacaggcagccgccgtgacgcaagggacgccatgcgtgccgctgccgaggtttccggtgcctcaccccatctacagcagaccggcgatggtacctccgccgccaagcaccaccaagctagctcaactgcatctggagctccaagcgcacccgtccaaggagagcatcgacgcagccatcggagatgttttagtgaagccatggctgccgcttccactggggctcaagccgccgtcgctcgacagcgtcatgtcggagctgcacaagcaaggcgtaccaaaaatcccaccggcggctgccaccaccaccggcgccaccggatgacatactatctcgtcgacaatacatgcatgtacaatagacggatgtctagtagtatagtagattgctgctagctagctagccgctagagtgtagtagcatatgcatgccttttttttcttcttctttttttgcccttattattaagctggctaatagcgattgagatggagcttgacacagatctgctagctagctatttgagggtttctcttgtatgctacctattgctgctgcttgctgctgagacaagtaatgtacgtacgcctgtgcaaacgacacatcggattgtattgtattactagttatatgaacaacaacaataataataggcacatgcatgcctgcctagtatgtgagctgctagctagctacatgcatgttgcgcattcctttcc

SEQ ID NO: 34 - DNA, 1748 bp, Artificial Sequence
  Description of Artificial Sequence; note = synthetic construct
ggcacgaggccgcacgaactgcatgccagtttaagttcagtgctcaaatcgggcctgcagcagaagcgctccagacctaacgtgccgttgcttccgccgcattgcgctgggctgaacttgcacgtagctggctgtggtgggtagagtggccgagatgcttgccgtgtcctccgcgaggtgcctcgccgacgacgcggtggagcagtgcgccgaggccgcgcccgtggagacggttgggggcgcggtggtggtggcggacctggacatagacttcgacttcaccgttgatgacatagactttggggatttcttcctccggctagaggacggagacgcgctgccggacctggaggtcgaccccgccgacatcttcactgatctcgaggcggcggccgcgggcgtcgaggagttgcaggaccagcaggtgccttgcgccgagctcttggccgccgtggaggacgtcggttcggtcagttcggccggtggtgtcatcgccgtagagaacgcggcgttcgctgaggcggggctaggagacgggaagcgagggtgcagtcaagccgaggtggacgaaagcatgggcggcggcgaccgccccgttgtgccggacgcaaaatcgccgtcgtcgacaacgtcgtcgtctaccgaggctgagagccgccacaagtcgtcaagcaagaactcccacgggaagaagaaagccaaggtggactggacgccggagctgcaccggaggttcgtgcaggcggtagagcagctcggcatcgacaaggcggtgccgtcgaggattctggagatcatggggatcaactcactcactcggcacaacatagcaagccatttgcagaagtaccggtctcaccggaagcacatgattgcgcgggaggcagaggcggcgagttggacccaacgacgacagatgtatgccgccggcggaccggctgcggccgtgaagaggcaggactcgaacatatggaccgtgccaaccatcggcttcgcgccggcgcaccctcctcctcctcctcctccttcaccggcagccatgcagcactacgtccggccgttgcacgtctggggtcaccccacgatggactcgccccggatgccaatgtggccgaggcacccaatgccccgcgccccgatgccggcgtgggttcccccgccgccgccgccgtccgacccggctttctggcaccacccttacatgagggggccagcagcgtatatgccgacccatgggactccttgcatggcaatgccgatggcaccgaaatttcctgctcctcctgtgcccgttgccatgccgtgtccagtctacacgcccccctccacgccatcagcgctggcgagcaagaaccagcaagaatcgcagctccagctacaagcacaaccatcaaacgagagcatagacgcggccatcggtgatgttttatccaaaccgtggctgccactgccgctggggctgaagcccccttcgttgggcagtgtcatgggcgagcttgaaaggcaaggcgtggccaatgtgccacaagcctgcgggtgagcgctggaggatgcatccgcctgcactccatgcatgactgctgctccgttcagagtaaccgctagcagcagcaccaacgacaacaacaacaaaatcgtcgacatgtcttttttccttagaacgttttgtggacctctctcttggtcagcctgtgcataatttcgatcaagagaaactttgttacttttcaaaaaaaaaaaaaaaaaaa

SEQ ID NO: 35 - DNA, 1965 bp, Artificial Sequence
  Description of Artificial Sequence; note = synthetic construct
gcacctagtcaccaccgcaccaaccgcgcgcgaccggccccccgtcagtcgcaagctgagcctgagcgagcaactgatgccgctagctatagctgctgcctgcagcacgcagcgcccgtaacctaacgtatcatttacttccattgcactgcacatcctgtgagttagttgtctgagtgagagctggacgtgctgcaccgagctcctggccgagatgcttgccgtgtcgccggcgatgtgccccgacattgaggaccgcgccgcggtggccggcgatgctggcatggaggtcgtcgggatgtcgtcggacgacatggatcagttcgacttctccgtcgatgacatagacttcggggacttcttcctgaggctggaggacggtgatgtgctcccggacctcgaggtcgacccggccgagatcttcaccgacttcgaggcaatcgcgacgagtggaggcgaaggtgtgcaggaccaggaggtgcccaccgtcgagctcttggcgcctgcggacgacgtcggtgtgctggatccgtgcggcgatgtcgtcgtcgggaaggagaacgcggcgtttgccggggctggagaggagaagggagggtgtaaccaggacgatgatgcgggggaagcgaatgccgacgatggagccgcggcggttgaggccaagtcttcgtcgccgtcatcgtcgacgtcgtcgtcgcaggaggctgagagccggcacaagtcatccagcaagagctcccatgggaagaagaaagcgaaggtggactggacgcctgagcttcaccggaggttcgtgcaggcggtggagcagctcggcatcgacaaggccgtgccgtcgaggatacttgagatcatggggatcgactcgctcacccggcacaacatagccagccatcttcagaagtaccggtcacacagaaaacacatgattgcgagagaggcggaggcagcgagttggacccaacggcggcagatttacgccgccggtggaggtgctgttgcgaagaggccggagtccaacgcgtggaccgtgccaaccattggcttccctcctcctccgccaccaccaccatcaccggctccgatgcaacattttgctcgcccgttgcatgtttggggccacccgacgatggacccgtcccgagttccagtgtggccaccgcggcacctcgttccccgtggcccggcgccaccatgggttccaccgccgccgccgtcggaccctgctttctggcaccacccttacatgagggggccagcacatgtgccaactcaagggacaccttgcatggcgatgcccatgccagctgcgagatttcctgctccaccggtgccaggagttgtcccgtgtccaatgtataggccattgactccaccagcactgacgagcaagaatcagcaggacgcacagcttcaactccaggttcaaccatcaagcgagagcatcgacgcagctatcggtgatgttttatcgaaaccgtggttgcctttgcctcttggactgaagccaccttcagtggacagtgtgatgggcgagctgcagaggcaaggcgtagcaaatgttcctccagcgtgtggatgatatttgcatgatagattgatggttccgttactgactgattgcatatctggtcagttcaccaaaaaatgggaaaattcgccgacatgtccttttatttttcttttggccctagcgtttcttggacatttcgtttaatttttaacttgtgcagagttcgaaagacatctttgtttcgactccgggagaaattaacctgcgtattgtaaatgtaaaatgtatttgccgtgagttctcagcagctaagaataaattaaataatggtcctccgagtagggtttacttgattttgcgtgttggtgttggttcaaacgctctaatatctaaattccgcattgtactattttcacaaaggaaagtagcccccgcaattgcacaggcttctt

SEQ ID NO: 36 - protein, 475 aa, Artificial Sequence
  Description of Artificial Sequence; note = synthetic construct
MLAVSPSPVRCADAEECGGGGASKEMEETAVGPVSDSDLDFDFTVDDIDFGDFFLRLDDGDDALPGLEVDPAEIVFADFEAIATAGGDGGVTDQEVPSVLPFADAAHIGAVDPCCGVLGEDNDAACADVEEGKGECDHADEVAAAGNNNSDSGEAGCGGAFAGEKSPSSTASSSQEAESRRKVSKKHSQGKKKAKVDWTPELHRRFVQAVEELGIDKAVPSRILEIMGIDSLTRHNIASHLQKYRSHRKHMLAREVEAATWTTHRRPMYAAPSGAVKRPDSNAWTVPTIGFPPPAGTPPRPVQHFGRPLHVWGHPSPTPAVESPRVPMWPRHLAPRAPPPPPWAPPPPADPASFWHHAYMRGPAAHMPDQVAVTPCVAVPMAAARFPAPHVRGSLPWPPPMYRPLVPPALAGKSQQDALFQLQIQPSSESIDAAIGDVLTKPWLPLPLGLKPPSVDSVMGELQRQGVANVPQACG

SEQ ID NO: 37 - protein, 461 aa, Artificial Sequence
  Description of Artificial Sequence; note = synthetic construct
MLAVSSARCLADDAVEQCAEAAPVETVGGAVVVADLDIDFDFTVDDIDFGDFFLRLEDGDALPDLEVDPADIFTDLEAAAAGVEELQDQQVPCAELLAAVEDVGSVSSAGGVIAVENAAFAEAGLGDGKRGCSQAEVDESMGGGDRPVVPDAKSPSSTTSSSTEAESRHKSSSKNSHGKKKAKVDWTPELHRRFVQAVEQLGIDKAVPSRILEIMGINSLTRHNIASHLQKYRSHRKHMIAREAEAASWTQRRQMYAAGGPAAAVKRQDSNIWTVPTIGFAPAHPPPPPPPSPAAMQHYVRPLHVWGHPTMDSPRMPMWPRHPMPRAPMPAWVPPPPPPSDPAFWHHPYMRGPAAYMPTHGTPCMAMPMAPKFPAPPVPVAMPCPVYTPPSTPSALASKNQQESQLQLQAQPSNESIDAAIGDVLSKPWLPLPLGLKPPSLGSVMGELERQGVANVPQACG

SEQ ID NO: 38 - protein, 455 aa, Artificial Sequence
  Description of Artificial Sequence; note = synthetic construct
MLAVSPAMCPDIEDRAAVAGDAGMEVVGMSSDDMDQFDFSVDDIDFGDFFLRLEDGDVLPDLEVDPAEIFTDFEAIATSGGEGVQDQEVPTVELLAPADDVGVLDPCGDVVVGKENAAFAGAGEEKGGCNQDDDAGEANADDGAAAVEAKSSSPSSSTSSSQEAESRHKSSSKSSHGKKKAKVDWTPELHRRFVQAVEQLGIDKAVPSRILEIMGIDSLTRHNIASHLQKYRSHRKHMIAREAEAASWTQRRQIYAAGGGAVAKRPESNAWTVPTIGFPPPPPPPPSPAPMQHFARPLHVWGHPTMDPSRVPVWPPRHLVPRGPAPPWVPPPPPSDPAFWHHPYMRGPAHVPTQGTPCMAMPMPAARFPAPPVPGVVPCPMYRPLTPPALTSKNQQDAQLQLQVQPSSESIDAAIGDVLSKPWLPLPLGLKPPSVDSVMGELQRQGVANVPPACG

SEQ ID NO: 39 - protein, 461 aa, Artificial Sequence
  Description of Artificial Sequence; note = synthetic construct
MLEVSTLRGPTSSGSKAEQHCGGGGGFVGDHHVVFPTSGDCFAMVDDNLLDYIDFSCDVPFFDADGDILPDLEVDTTELLAEFSSTPPADDLLAVAPFGADDQPAAAVAQEKPSSSLEQTCGDDKGVAVAAARRKLQTTTTTTTTEEEDSSPAGSGANKSSASAEGHSSKKKSAGKNSNGGKRKVKVDWTPELHRRFVQAVEQLGIDKAVPSRILEIMGTDCLTRHNIASHLQKYRSHRKHLMAREAEAATWAQKRHMYAPPAPRTTTTTDAARPPWVVPTTIGFPPPRFCRPLHVWGHPPPHAAAAEAAAATPMLPVWPRHLAPPRHLAPWAHPTPVDPAFWHQQYSAARKWGPQAAAVTQGTPCVPLPRFPVPHPIYSRPAMVPPPPSTTKLAQLHLELQAHPSKESIDAAIGDVLVKPWLPLPLGLKPPSLDSVMSELHKQGVPKIPPAAATTTGATG

SEQ ID NO: 40 - protein, 539 aa, Artificial Sequence
  Description of Artificial Sequence; note = synthetic construct
MLEVSTLRSPKADQRAGVGGHHVVGFVPAPPSPADVADEVDAFIVDDSCLLEYIDFSCCDVPFFHADDGDILPDLEVDPTELLAEFASSPDDEPPPTTSAPGPGEPAAAAGAKEDVKEDGAAAAAAAAAADYDGSPPPPRGKKKKDDEERSSSLPEEKDAKNGGGDEVLSAVTTEDSSAGAAKSCSPSAEGHSKRKPSSSSSSAAAGKNSHGKRKVKVDWTPELHRRFVQAVEQLGIDKAVPSRILELMGIECLTRHNIASHLQKYRSHRKHLMAREAEAASWTQKRQMYTAAAAAAAVAAGGGPRKDAAAATAAVAPWVMPTIGFPPPHAAAMVPPPPHPPPFCRPPLHVWGHPTAGVEPTTAAAPPPPSPHAQPPLLPVWPRHLAPPPPPLPAAWAHGHQPAPVDPAAYWQQQYNAARKWGPQAVTPGTPCMPPPLPPAAMLQRFPVPPVPGMVPHPMYRPIPPPSPPQGNKLAALQLQLDAHPSKESIDAAIGDVLVKPWLPLPLGLKPPSLDSVMSELHKQGIPKVPPAASGAAG

SEQ ID NO: 41 - protein, 384 aa, Artificial Sequence
  Description of Artificial Sequence; note = synthetic construct
MLTVSPAPVLIGNNSKDTYMAADFADFTTEDLPDFTTVGDFSDDLLDGIDYYDDLFIGFDGDDVLPDLEIDSEILGEYSGSGRDEEQEMEGNTSTASETSERDVGVCKQEGGGGGDGGFRDKTVRRGKRKGKKSKDCLSDENDIKKKPKVDWTPELHRKFVQAVEQLGVDKAVPSRILEIMNVKSLTRHNVASHLQKYRSHRKHLLAREAEAASWNLRRHATVAVPGVGGGGKKPWTAPALGYPPHVAPMHHGHFRPLHVWGHPTWPKHKPNTPASAHRTYPMPAIAAAPASWPGHPPYWHQQPLYPQGYGMASSNHSSIGVPTRQLGPTNPPIDIHPSNESIDAAIGDVISKPWLPLPLGLKPPSVDGVMTELQRQGVSNVPP

SEQ ID NO: 42 - DNA, 23 bp, Artificial Sequence
  Description of Artificial Sequence; note = synthetic construct
tacaattattccctagagaccat

SEQ ID NO: 43 - DNA, 23 bp, Artificial Sequence
  Description of Artificial Sequence; note = synthetic construct
tacaattattccctagagaccat

SEQ ID NO: 44 - DNA, 24 bp, Artificial Sequence
  Description of Artificial Sequence; note = synthetic construct
cgtaagcttggctgtggtgggtag

SEQ ID NO: 45 - DNA, 26 bp, Artificial Sequence
  Description of Artificial Sequence; note = synthetic construct
ttgttgtcgatatcgtgctgctgcta

SEQ ID NO: 46 - DNA, 24 bp, Artificial Sequence
  Description of Artificial Sequence; note = synthetic construct
tataaacccgtcgactcattcatt

SEQ ID NO: 47 - DNA, 24 bp, Artificial Sequence
  Description of Artificial Sequence; note = synthetic construct
gggcaagtcattcatatctaagga

SEQ ID NO: 48 - DNA, 21 bp, Artificial Sequence
  Description of Artificial Sequence; note = synthetic construct
cggccgcgtaaactaaaccta

SEQ ID NO: 49 - DNA, 24 bp, Artificial Sequence
  Description of Artificial Sequence; note = synthetic construct
tcccggcaagtatcataacaagta

SEQ ID NO: 50 - DNA, 23 bp, Artificial Sequence
  Description of Artificial Sequence; note = synthetic construct
agtgccccaggtcgagtttgatg

SEQ ID NO: 51 - DNA, 24 bp, Artificial Sequence
  Description of Artificial Sequence; note = synthetic construct
tcccgcctttctttagttgattgg

SEQ ID NO: 52 - DNA, 23 bp, Artificial Sequence
  Description of Artificial Sequence; note = synthetic construct
gttggcactaccggcgatactta

SEQ ID NO: 53 - DNA, 24 bp, Artificial Sequence
  Description of Artificial Sequence; note = synthetic construct
acacccaaccggaataccaacaac

SEQ ID NO: 54 - DNA, 24 bp, Artificial Sequence
  Description of Artificial Sequence; note = synthetic construct
gtggaatacttgccgtgttaggat

SEQ ID NO: 55 - DNA, 24 bp, Artificial Sequence
  Description of Artificial Sequence; note = synthetic construct
cacgagccaccatgaaagagttat

SEQ ID NO: 56 - DNA, 20 bp, Artificial Sequence
  Description of Artificial Sequence; note = synthetic construct
ccgcgggcgtcgaggagttg

SEQ ID NO: 57 - DNA, 24 bp, Artificial Sequence
  Description of Artificial Sequence; note = synthetic construct
cggcggcatacatctgtcgtcgtt